(19) United States
(12) Patent Application Publication
(10) Pub. No.: US 2014/0155397 A1
(43) Pub. Date: Jun. 5, 2014

(54) EMT SIGNATURES AND PREDICTIVE MARKERS AND METHOD OF USING THE SAME

(71) Applicants: John V Heymach, Pearland, TX (US); Jing Wang, Pearland, TX (US); Lauren Averett Byers, Houston, TX (US); Kevin R. Coombes, Columbus, OH (US); John D. Minna, Dallas, TX (US); Luc Girard, Dallas, TX (US)

(72) Inventors: John V Heymach, Pearland, TX (US); Jing Wang, Pearland, TX (US); Lauren Averett Byers, Houston, TX (US); Kevin R. Coombes, Columbus, OH (US); John D. Minna, Dallas, TX (US); Luc Girard, Dallas, TX (US)

(73) Assignee: Board of Regents, Austin, TX (US)

(21) Appl. No.: 14/009,208

(22) PCT Filed: Apr. 2, 2012
(86) PCT No.: PCT/US12/31873

§ 371 Date: Oct. 1, 2013

Related U.S. Application Data
(60) Provisional application No. 61/470,625, filed on Apr. 1, 2011.
(60) Provisional application No. 61/472,098, filed on Apr. 5, 2011.

(51) Int. Cl. C12Q 1/68; A61K 31/5377; A61K 31/519; A61K 31/517

(52) U.S. Cl. CPC C12Q 1/6886; A61K 31/517; A61K 31/5377; A61K 31/519
USPC 514/234.5; 506/9; 514/266.4; 514/252.16

(57) ABSTRACT

EMT signatures and markers useful for characterizing the status of epithelial cancers and for predicting drug responses in patients having non-small cell lung cancer are provided together with methods of using the same.

### STATEMENT REGARDING FEDERALLY SPONSORED RESEARCH OR DEVELOPMENT

This invention was made with government support under awarded under P50 CA070907 by the National Institutes of Health/National Cancer Institute, and under W81XWH-07-1-0306 and W81XWH-06-1-0303 by the Department of Defense. The government has certain rights in the invention.

### FIELD OF INVENTION

This invention relates generally to EMT signatures and predictive markers for successful drug therapy, and more particularly, gene expression signatures and markers useful for characterizing the status of epithelial cancers and for predicting drug responses in patients having non-small cell lung cancer.

### CROSS-REFERENCE TO RELATED APPLICATION

This application claims the benefit of and priority in U.S. Patent Application Ser. Nos. 61/470,625 filed on Apr. 1, 2011 and 61/472,098 filed Apr. 5, 2011. The applications are herein incorporated by reference.

### THE NAMES OF THE PARTIES TO A JOINT RESEARCH AGREEMENT

None.

### REFERENCE TO SEQUENCE LISTING BACKGROUND OF THE INVENTION

None.

### BACKGROUND OF THE INVENTION

Epithelial-mesenchymal transition (“EMT”) has been associated with metastatic spread and EGFR inhibitor resistance. However, currently, there is no standard method for assessing EMT. Hence, there is an unmet need for therapeutic strategies targeting mesenchymal cells and overcoming EMT-associated drug resistance. Furthermore, to date, EGFR mutation is the only validated marker for identifying and predicting a benefit in patients with wild type EGFR mutation in non-small cell lung cancer.

Signatures and biomarkers are needed to select patients that will experience greater benefit from a specific treatment regimen for non-small cell lung cancer and other cancers, potentially sparing patients who are less likely to benefit from receiving toxic therapy.

### BRIEF SUMMARY OF THE INVENTION

Epithelial-mesenchymal transition (“EMT”) gene expression signatures are provided herein. These signatures are useful for characterizing the status of epithelial cancers and for predicting certain drug responses in patients having non-small cell lung cancer (“NSCLC”). The gene signatures as well as certain individual biomarkers disclosed herein can be used to identify which NSCLC patients may benefit from certain drug treatments. The signatures may also be useful for predicting response to EGFR inhibitors in NSCLC as well as other tumor types. In addition, EGFR mutations could be used in conjunction with these EMT signatures and other biomarkers (sometimes referred to herein as “markers”) to identify patients at greater risk for relapse or metastatic spread after definitive (e.g. surgery, radiation) therapy.

As taught herein, we confirmed that certain signatures are associated with shorter progression and overall survival. These signatures together with other markers could be useful for improving the selection of patients likely to respond to a given treatment, particularly for NSCLC patients treated with EGFR inhibitors. The signatures also may be used for selecting patients to receive cisplatin-based chemotherapy.

The EMT signatures presented herein were developed using non-small cell lung cancer cell lines. These signatures been have validated using independent gene expression platforms, for NSCLC lines and head and neck cell lines. Clinical validation was performed using several clinical datasets including the BATTLE study, which confirmed the signature is as a marker of erlotinib resistance, and a set of head and neck patients who received PORT (“post-operative radiotherapy”).

The EMT gene expression signatures disclosed herein can also accurately classify cell lines as epithelial or mesenchymal-like across microarray platforms and several cancer types. Furthermore, as taught herein Axl and LCN2 have been identified as a novel EMT markers in NSCLC and Head and Neck Cancer (“HNC”). Hence, the EMT signature is a reliable predictor of erlotinib resistance and is more accurate than single mRNA or protein markers such as E-cadherin.

### BRIEF DESCRIPTION OF THE DRAWINGS

FIG. 1 shows that the EMT gene expression signature described herein separates NSCLC cell lines into distinct epithelial-like and mesenchymal-like groups independent of microarray platform.

FIGS. 2A, 2B and 2C show the validation of the EMT signature across platforms and in independent testing set of cell lines.

FIGS. 3A, 3B and 3C show the results from the integrated analysis of protein expression and the EMT signature.

FIGS. 4A, 4B, 4C, 4D, 4E and 4F show that mesenchymal lines are resistant to EGFR inhibition and PI3K pathway inhibition but sensitive to Axl inhibition by SGI-7079.

FIG. 5 shows the EMT signature predicts resistance to EGFR and PI3K inhibitors.

FIGS. 6A and 6B show that the EMT signature predicts erlotinib sensitivity better than CDH1 or VIM probes.

FIGS. 7A, 7B, and 7C show the improved 8-week disease control in BATTLE patients with epithelial signatures treated with erlotinib.

FIGS. 8A and 8B show that different probes for the same gene vary within and across microarray platforms.

FIGS. 9A, 9B, and 9C show that CDH1 probes vary in their accuracy and dynamic range.

FIG. 10 shows the structure of pyrrolopyrimidine AXL inhibitor SGI-7079.

FIGS. 11A and 11B show the results of signature testing in independent NSCLC and HNC cell lines on the Illumina v3 microarray platform.

FIGS. 12A, 12B, 12C and 12D show the improved 8-week disease control in BATTLE patients with epithelial signatures treated with erlotinib.

FIGS. 13A, 13B, and 13C show further results from the integrated analysis of protein expression and the EMT signature.

FIG. 14 shows further scatter plot data of the experiment of different probes across microarray platforms.

FIGS. 15A, 15B, and 15C shows that the EMT signature predicts disease control in advanced, pretreated NSCLC patients with wildtype EGFR and KRAS following treatment with erlotinib.

FIG. 16A shows the correlation between all cell lines with erlotinib IC50 and different signatures. FIG. 16B shows the correlation between EGFR wild type cell lines with erlotinib IC50 and different signatures. FIG. 16C shows the correlation between EGFR and KRAS wild type cell lines with erlotinib IC50 and different signatures.

FIG. 17 shows further results from the integrated analysis of protein expression and the EMT signature.

FIGS. 18A, 18B, and 18C show erlotinib sensitivity data for cell lines and clinical samples.

FIG. 19A is a dot plot between the disease control groups of the EMT signature using the selected genes in all evaluable erlotinib treated patients. FIG. 19B is a dot plot between the disease control groups of the EMT signature using the selected genes in EGFR wild type evaluable erlotinib treated patients. FIG. 19C is a dot plot between the disease control groups of the EMT signature using the selected genes in EGFR and KRAS wild type evaluable erlotinib treated patients. FIG. 19D shows the survival plots of the study.

FIG. 20 shows the results of a training set (Affymetrix) of 54 NSCLC cell lines for the refined EMT signature.

FIG. 21 shows the 35 genes in the refined EMT signature as overexpressed in mesenchymal, epithelia and KRAS mutated mesenchymal and in epithelial cells.

FIG. 22 is a plot of the first two principal components in the affy lung cancer data.

FIG. 23 shows the results of the cross-platform testing of the Illumina array.

FIG. 24 is a chart showing the histologies between the groups.

FIG. 25 shows 100% concurrence between E- and M-classifications with the 76 and 35 gene signatures.

FIG. 26 is a diagram showing the multipronged approaches to developing gene expression signatures for BATTLE.

FIG. 27 is a chart summarizing the predictive value of the EGFR, KRAS, EMT and 5 gene WEE signatures.

FIG. 28 shows that genes are differentially expressed with a fold-change greater than 2 and overlapping between the 3 training sets.

FIG. 29 shows that the EGFR index is associated with EGFR, but not KRAS, mutations.

FIGS. 30A and 30B show that the EGFR signature predicts EGFR mutation status in validation sets of tumors and cell lines.

FIG. 31 shows that the EGFR signature is associated with sensitivity to erlotinib in vitro.

FIG. 32 show that EGFR signature is associated with relapse free survival in patients with wild-type EGFR.

FIG. 33 is a chart showing EGFR signature is associated with relapse-free survival patients with wild-type EGFR.

FIGS. 34A and 34B show EGFR mutants and KRAS mutants in BATTLE samples.

FIG. 35 shows EGFR signature in BATTLE samples.

FIGS. 36A and 36B provides the results of progression-free survival of patients with wild-type EGFR being treated with erlotinib and the 8-weeks disease control of patients with wild-type EGFR with rating the signature value associated with the different treatments of erlotinib, sorafenib and vandetanib.

FIGS. 37A and 37B provides the results of progression-free survival of patients with wild-type EGFR being treated with sorafenib and the 8-weeks disease control of patients with wild-type EGFR with rating the signature value associated with the different treatments of erlotinib, sorafenib and vandetanib.

FIGS. 38A and 38B show that the EGFR signature is associated with decreased mitosis genes and increased receptor-mediated endocytosis genes.

FIG. 39 depicts the Kras signature and clinical outcome in BATTLE.

FIGS. 40A-D show that MACC1 is overexpressed in mutant EGFR cells.

FIGS. 41A, 40B, and 40C show that the MACC1 gene and protein expression are correlated with MET expression in cell lines.

FIGS. 42A and 42B show that MACC1 inhibition down-regulates total MET and phospho-MET in HCC827, a mutant EGFR cell line.

FIGS. 43A and 43B show that the EMT signature is predictive of DC in BATTLE patients with EGFR and KRAS treated with erlotinib.

FIG. 44 shows that the EMT gene expression signature predicts outcome in head and neck small cell cancer (“HNSCC”) patients treated with adjuvant RT.

FIGS. 45A, 45B, 45C and 45D show that the 5-gene signature including LCN2 is predictive of benefit for erlotinib in patients with wild-type EGFR.

FIGS. 46A and 46B show the validation of the 5-gene signature in a large panel of cell lines.

FIGS. 47A and 47B show that LCN2 is associated with erlotinib sensitivity in vitro in cells with wild-type EGFR.

FIGS. 48A and 48B show that LCN2 promoter methylation is associated with erlotinib sensitivity in vitro.

FIGS. 49A, 49B, 49C and 49D show that LCN2 promoter methylation is associated with erlotinib sensitivity in vitro.

FIGS. 50A, 50B, 50C and 50D show that the 5-gene signature and LCN2 are associated with erlotinib sensitivity in vitro.

FIG. 51 shows the sorafenib 15-gene signature and results from the 8-week disease control study.

FIG. 52 shows the results of the validation of the 5-gene signature in a large panel of cell lines.

FIG. 53 shows the gene expression distribution of the 5 genes in 108 NSCLC cell lines.

FIGS. 54A and 54B show that LCN2 is correlated with sensitivity to erlotinib.

FIGS. 55A and 55B show that genes correlated with lipocalin-2 (“LCN2”) are associated with sensitivity to gefitinib.

FIGS. 56A and 56B show that LCN2 expression is correlated with E-cadherin and epithelial phenotype.

FIG. 57 shows that LCN2 gene expression may be regulated through promoter methylation.

FIG. 58 describes how AXL is overexpressed in mesenchymal cells at the mRNA and protein levels.

FIG. 59 lists the probes representing 76 unique bimodally distributed genes that correlated with E-cadherin (CDHJ), vimentin (VIM), N-cadherin (CDH2), and/or fibronectin 1 (FN1) and identified in the NSCLC training set

### DETAILED DESCRIPTION OF THE INVENTION

Epithelial-mesenchymal transition (“EMT”) is a biological program observed in several epithelial cancers including non-small lung cancer cells (“NSCLC”). EMT is associated with loss of cell adhesion molecules such as E-cadherin and increased invasion, migration, and proliferation in epithelial cancers. Huber M. A., et al., Molecular Requirements for Epithelial-Mesenchymal Transition During Tumor Progression, Curr Opin Cell Biol. 17:548-58 (2005); Thiery J. P., Epithelial-Mesenchymal Transitions in Tumour Progression. Nature Rev. 2:442-54 (2002); Thiery J. P., et al., Epithelial-Mesenchymal Transitions in Development and Disease, Cell 139:871-90 (2009); Hugo H., et al., Epithelial-Mesenchymal and Mesenchymal-Epithelial Transitions in Carcinoma Progression, J Cell Physiol. 213:374-83 (2007).

Previous profiling and mutational analyses have demonstrated the molecular heterogeneity of non-small cell lung cancer. For EGFR mutant and EML4-ALK fusion subgroups, mutation status predicts response to therapy with EGFR inhibitors or ALK inhibitors, respectively. Unfortunately only a minority of patients express these markers, with EGFR mutations detected in ˜10-15% of lung adenocarcinomas and EML4-ALK fusions in ˜4%. Koivunen, J. P., et al., EML4-ALK Fusion Gene and Efficacy of an ALK Kinase Inhibitor in Lung Cancer, Clin Cancer Res. 14:4275-83 (2008); Pao, W., et al., EGF Receptor Gene Mutations are Common in Lung Cancers From “Never Smokers” and are Associated With Sensitivity Of Tumors To Gefitinib And Erlotinib, Proc Natl Acad Sci USA 101:13306-11 (2004); Lynch, T. J., et al., Activating Mutations In The Epidermal Growth Factor Receptor Underlying Responsiveness Of Non-Small-Cell Lung Cancer To Gefitinib, N Engl J Med. 350:2129-39 (2004); Paez, J. G., et al., EGFR Mutations in Lung Cancer: Correlation With Clinical Response To Gefitinib Therapy, Science 304:1497-500 (2004); Tokumo, M., et al., The Relationship Between Epidermal Growth Factor Receptor Mutations And Clinicopathologic Features In Non-Small Cell Lung Cancers, Clin Cancer Res. 11:1167-73 (2005); Cappuzzo, F., et al., Epidermal Growth Factor Receptor Gene and Protein and Gefitinib Sensitivity In Non-Small-Cell Lung Cancer, J Natl Cancer Inst. 97:643-55 (2005); Soda, M., et al., Identification of the Transforming EML4-ALK Fusion Gene in Non-Small-Cell Lung Cancer, Nature 448:561-6 (2007).

For the majority of patients with wild-type EGFR, only a certain subgroup appears to benefit from EGFR inhibitor treatment. However, prior to the present discoveries, there were no validated markers for identifying these patients. Bell D. W., et al., Epidermal Growth Factor Receptor Mutations and Gene Amplification in Non-Small-Cell Lung Cancer: Molecular Analysis of the IDEAL/INTACT Gefitinib Trials, J Clin Oncol. 23:8081-92 (2005); Zhu C. Q., et al., Role of KRAS and EGFR as Biomarkers of Response to Erlotinib in National Cancer Institute of Canada Clinical Trials Group Study BR.21, J Clin Oncol. 26:4268-75 (2008); Mok T. S., et al., Gefitinib or Carboplatin-Paclitaxel in Pulmonary Adenocarcinoma, N Engl J Med. 361:947-57 (2009).

Thus, presented herein are gene expression signatures and other validated predictive markers to accurately predict response to EGFR-targeted therapy in patients with wild-type EGFR mutation status, as well as for other targeted therapies, and that can help identify potential strategies for improving the efficacy of these agents.

As used herein, gene expression signatures are sometimes referred to herein as “signatures,” “gene signatures,” “EMT gene signatures,” “signature genes” “EMT signature genes” or “EMT signatures,” or, in the singular as a “signature,” “gene signature,” “EMT gene signature,” “signature gene” “EMT signature gene” or “EMT signature.”

Mesenchymal markers have been associated with limited responses to EGFR inhibitors, whereas an epithelial phenotype is associated with response even in patients without EGFR receptor mutations. Yauch R. L., et al., Epithelial Versus Mesenchymal Phenotype Determines In Vitro Sensitivity and Predicts Clinical Activity of Erlotinib in Lung Cancer Patients, Clin Cancer Res. 11:8686-98 (2005); Thomson S., et al., Epithelial to Mesenchymal Transition is a Determinant of Sensitivity of Non-Small-Cell Lung Carcinoma Cell Lines and Xenografts to Epidermal Growth Factor Receptor Inhibition, Cancer Res. 65:9455-62 (2005); Frederick B. A., et al., Epithelial to Mesenchymal Transition Predicts Gefitinib Resistance in Cell Lines of Head and Neck Squamous Cell Carcinoma and Non-Small Cell Lung Carcinoma, Mol Cancer Ther. 6:1683-91 (2007); Nikolova D. A., et al., Cetuximab Attenuates Metastasis and U-PAR Expression in Non-Small Cell Lung Cancer: U-PAR and E-Cadherin are Novel Biomarkers of Cetuximab Sensitivity, Cancer Res. 69:2461-70 (2009).

For example, high E-cadherin and low vimentin/fibronectin (i.e., an epithelial phenotype) has been associated with erlotinib sensitivity in cell lines and xenografts with wild-type EGFR. Thomson S., et al., Epithelial to Mesenchymal Transition is a Determinant of Sensitivity of Non-Small-Cell Lung Carcinoma Cell Lines and Xenografts to Epidermal Growth Factor Receptor Inhibition, Cancer Res. 65:9455-62 (2005). Clinically, E-cadherin protein expression has been associated with longer time to progression and a trend toward longer overall survival following combination erlotinib/chemotherapy. Yauch R. L., et al., Epithelial Versus Mesenchymal Phenotype Determines In Vitro Sensitivity and Predicts Clinical Activity of Erlotinib in Lung Cancer Patients, Clin Cancer Res. 11:8686-98 (2005). The ability to identify tumors that have not undergone EMT may help identify patients most likely to benefit from EGFR inhibition, particularly in patients with wild type EGFR. In addition, targeting EMT or EMT-associated resistance pathways may reverse or prevent acquisition of EGFR inhibitor resistance, as illustrated by one study in which restoration of an epithelial phenotype in mesenchymal NSCLC cell lines restored sensitivity to the EGFR inhibitor gefitinib. Witta S. E., et al., Restoring E-Cadherin Expression Increases Sensitivity to Epidermal Growth Factor Receptor Inhibitors in Lung Cancer Cell Lines, Cancer Res. 66:944-50 (2006). Although a number of markers have been associated with EMT and EMT signatures have been described in other cancer types, there is no validated signature in NSCLC that can identify tumors that have undergone EMT.

In non-small cell lung cancer (“NSCLC”), EMT is associated with worse prognosis and resistance to EGFR inhibitors. Despite the clinical implications, no gold standard exists for classifying a cancer as epithelial or mesenchymal. Our goal was to develop robust, platform-independent EMT gene expression signatures and test the correlation of these signatures with drug response.

In one aspect, we conducted analysis of an integrated gene expression, proteomic, and drug response using cell lines and tumors from non-small cell lung cancer patients. A 76-gene EMT signature was developed and validated using gene expression profiles from four microarray platforms of NSCLC cell lines and patients treated in the BATTLE (“Biomarker-integrated Approaches of Targeted Therapy for Lung Cancer Elimination”) study, and potential therapeutic targets associated with EMT were identified.

We found mesenchymal cells demonstrated significantly greater resistance to EGFR and PI3K/Akt pathway inhibitors, independent of EGFR mutation status, but not to sorafenib. Mesenchymal cells expressed increased levels of the receptor tyrosine kinase Axl and showed a trend towards greater sensitivity to the Axl inhibitor SGI-7079. The combination of SGI-7079 with erlotinib reversed erlotinib resistance in mesenchymal lines expressing Axl.

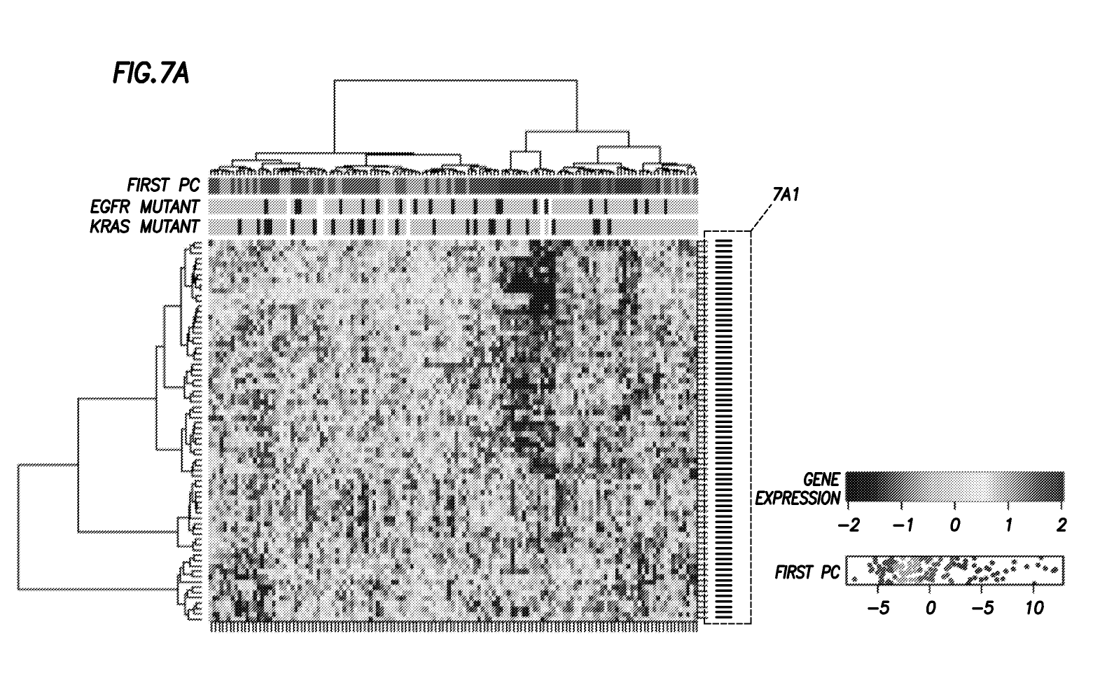

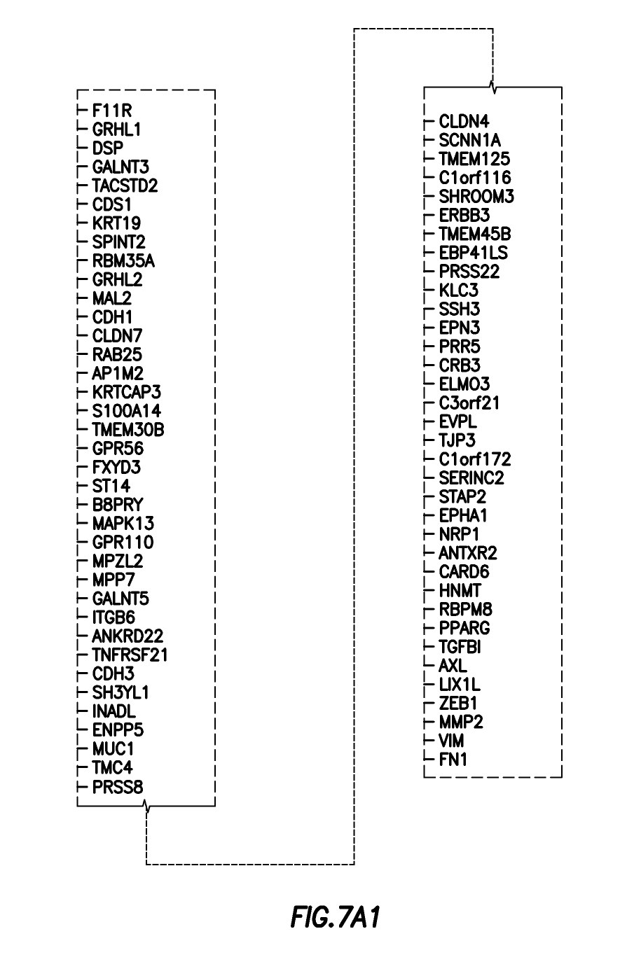

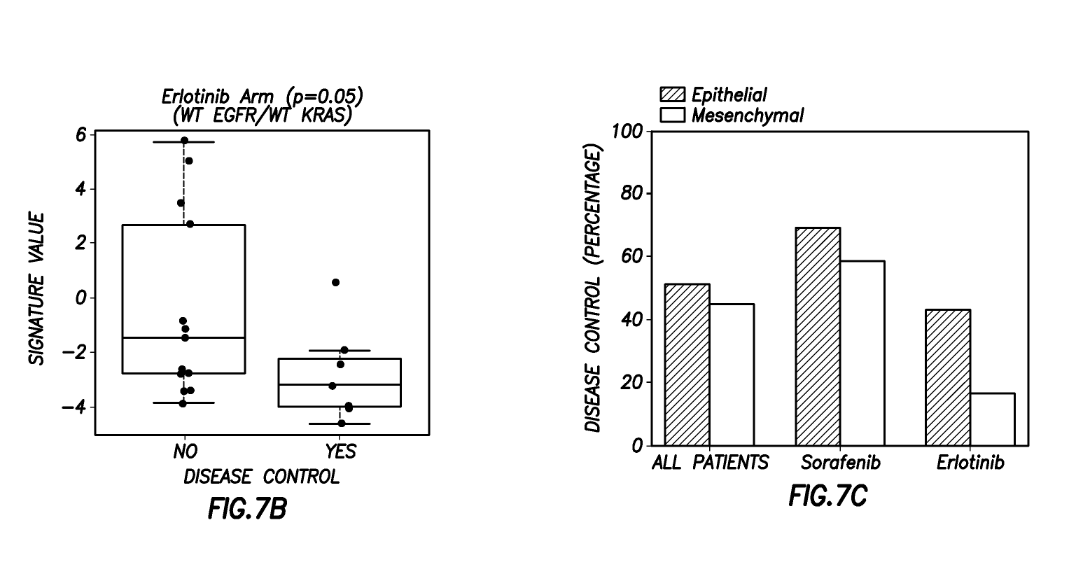

In NSCLC patients with non-mutated EGFR, the EMT signature predicted 8-week disease control in patients receiving erlotinib, but not other therapies. See, FIGS. 7 & 12. As a result of this study alone, we have developed a robust EMT signature that predicts resistance to EGFR and PI3K/Akt inhibitors and highlights different patterns of drug responsiveness for epithelial and mesenchymal cells.

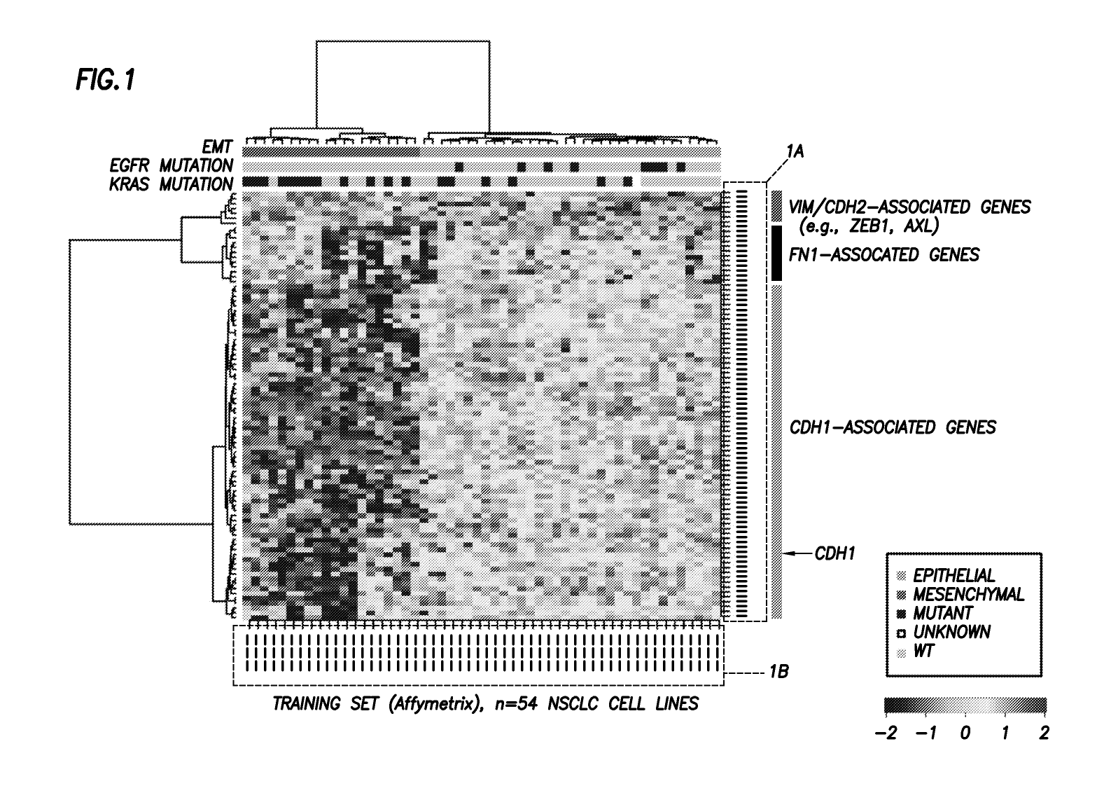

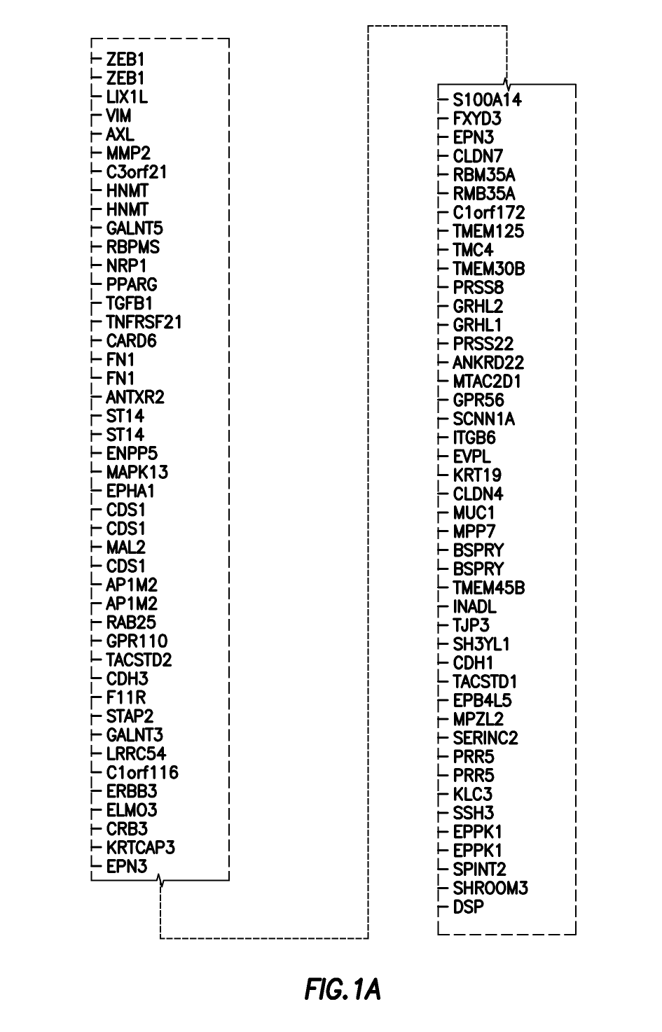

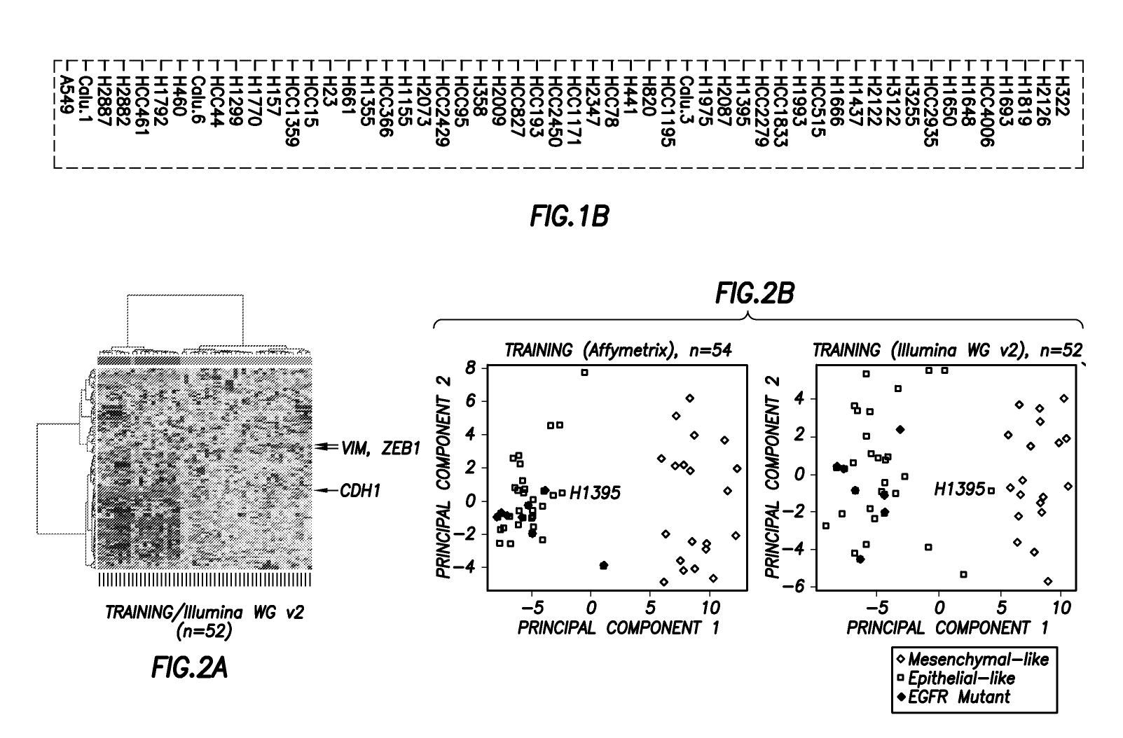

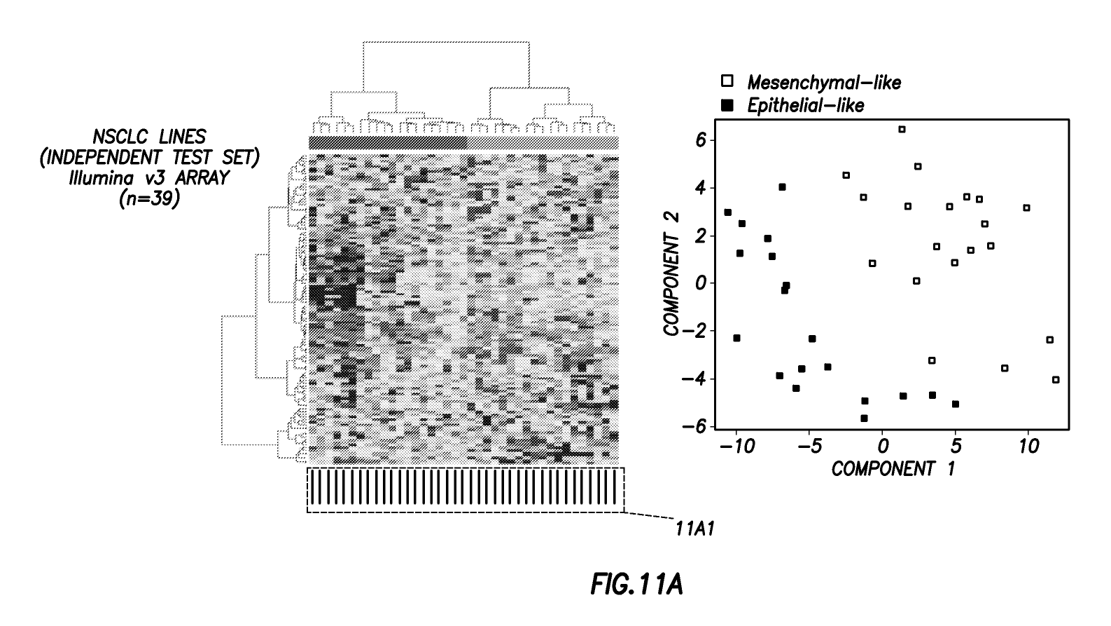

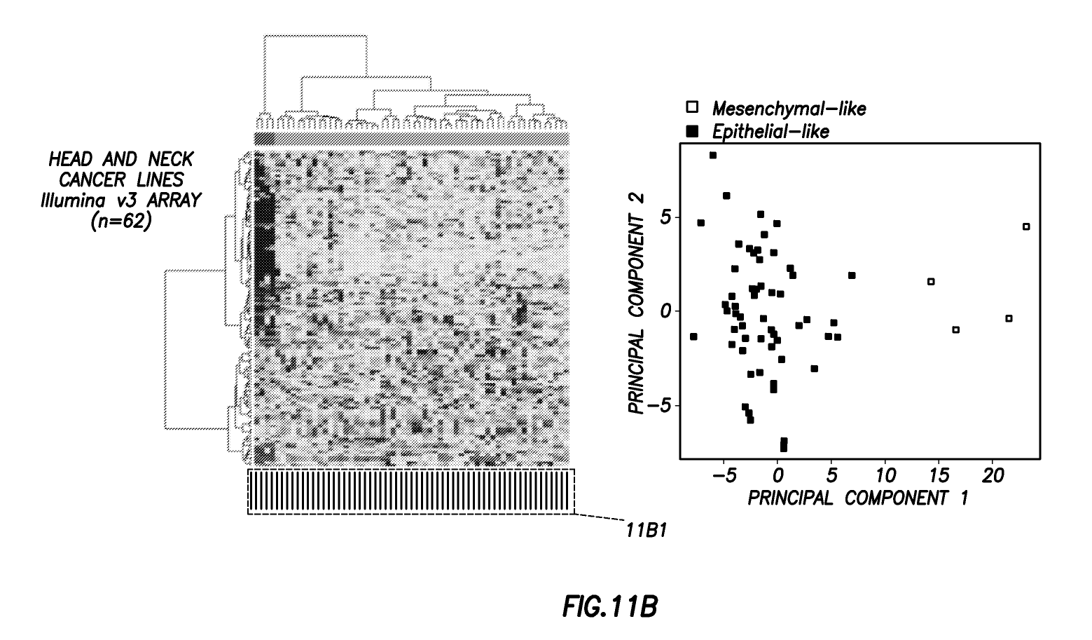

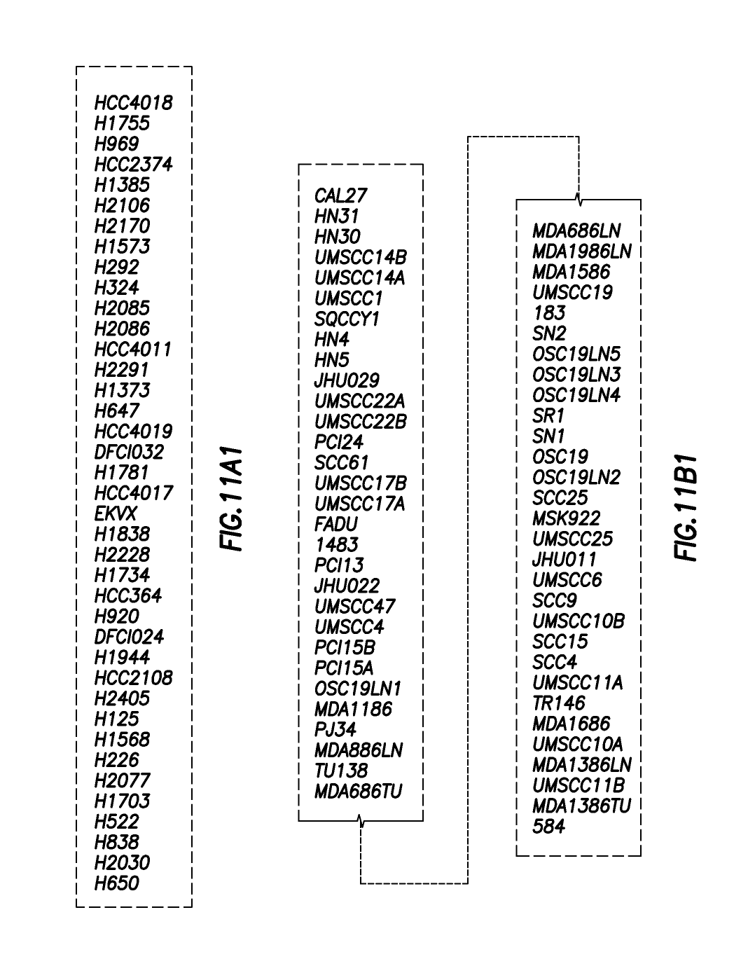

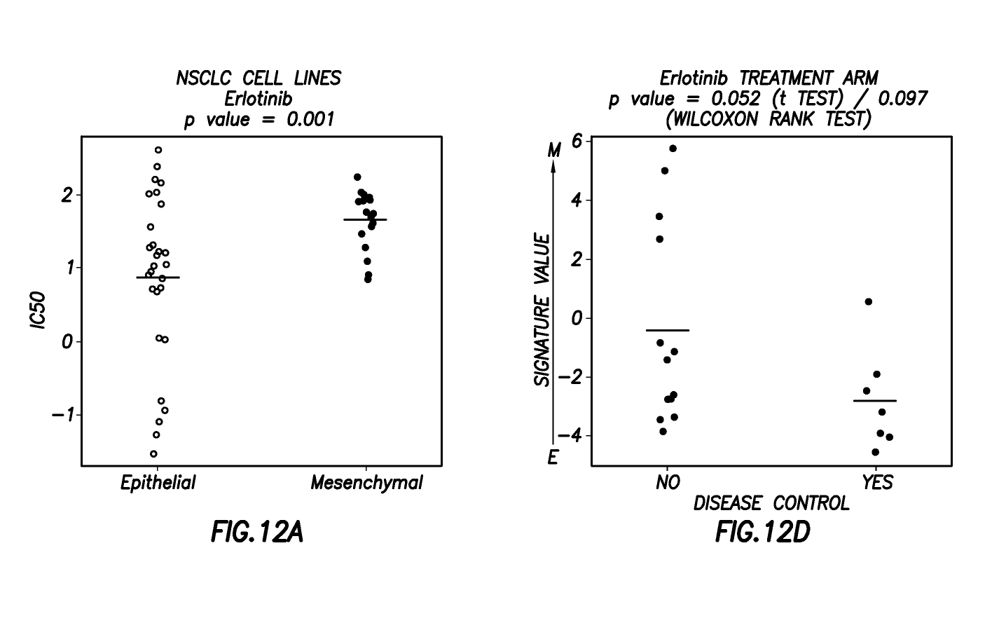

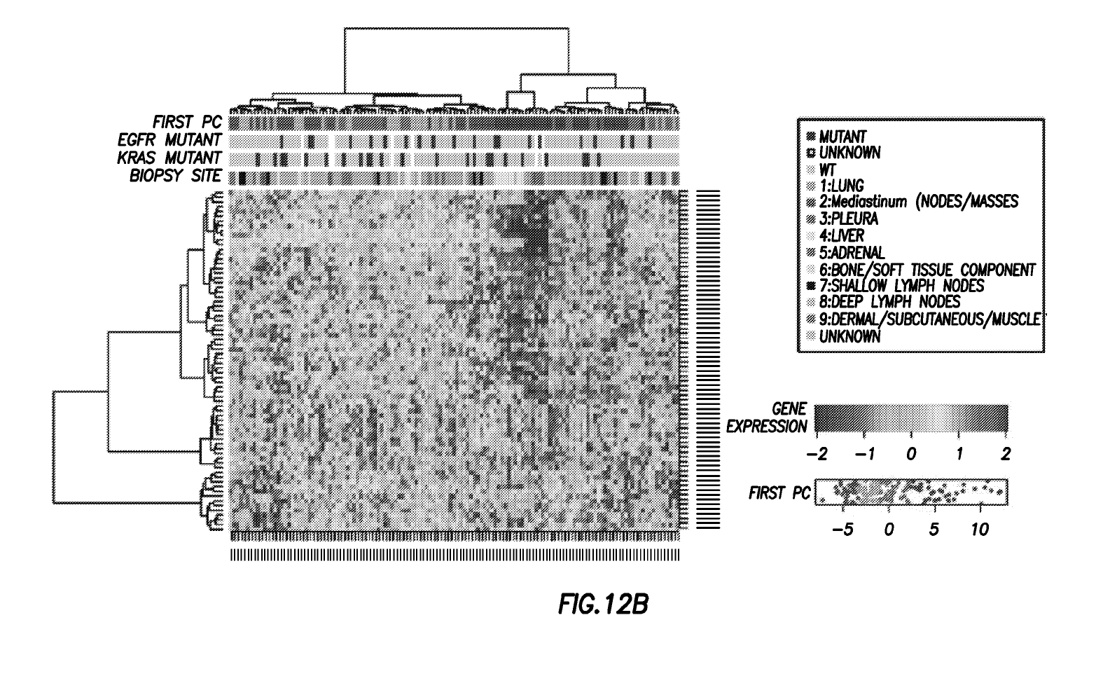

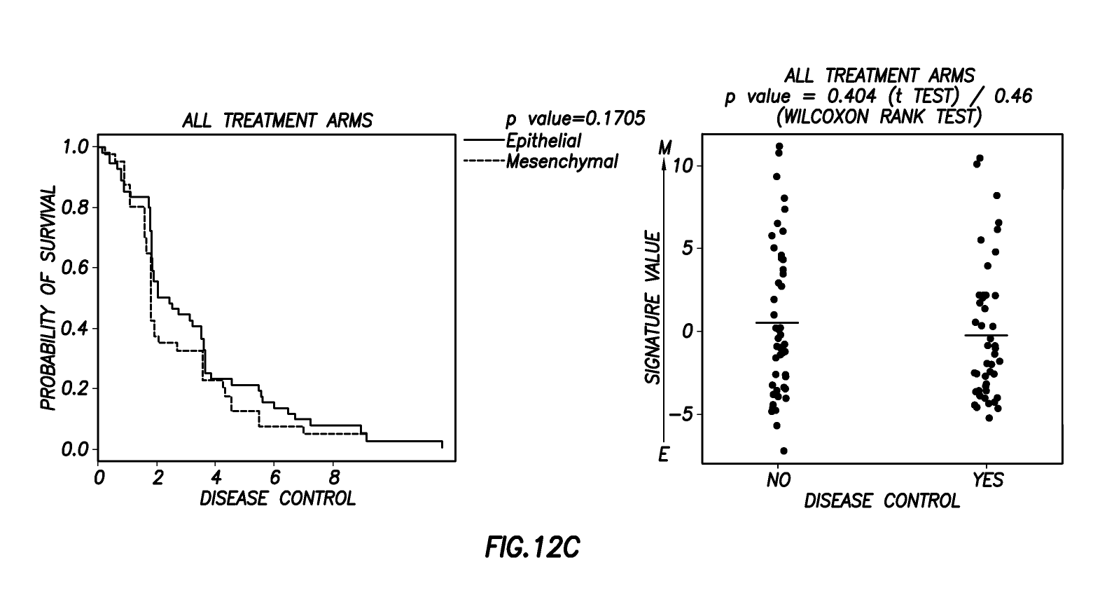

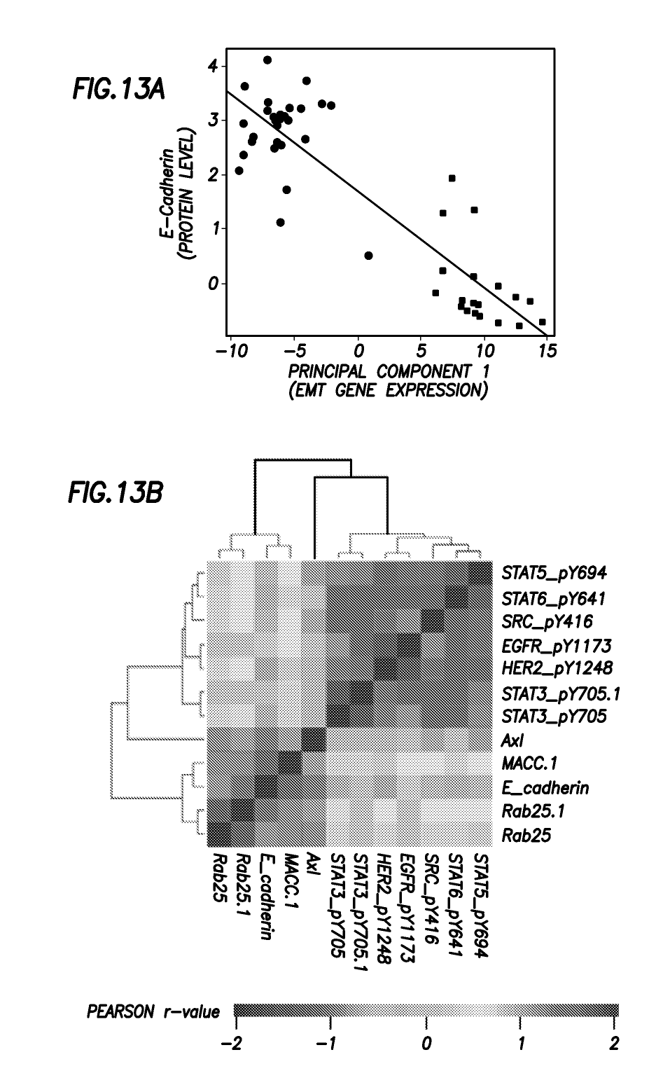

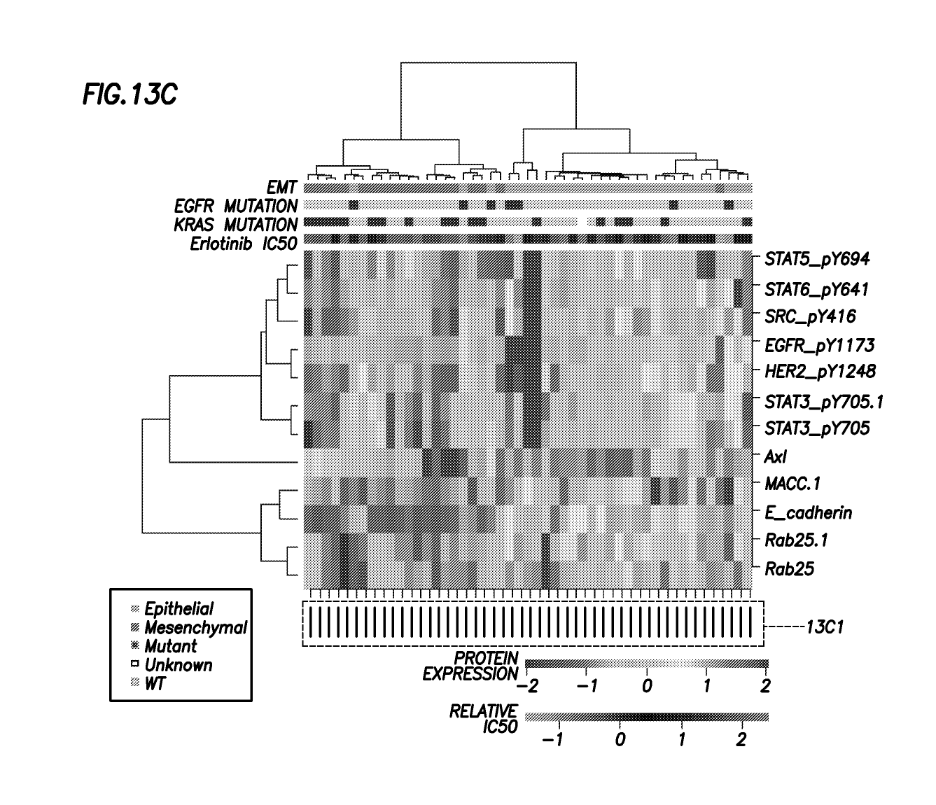

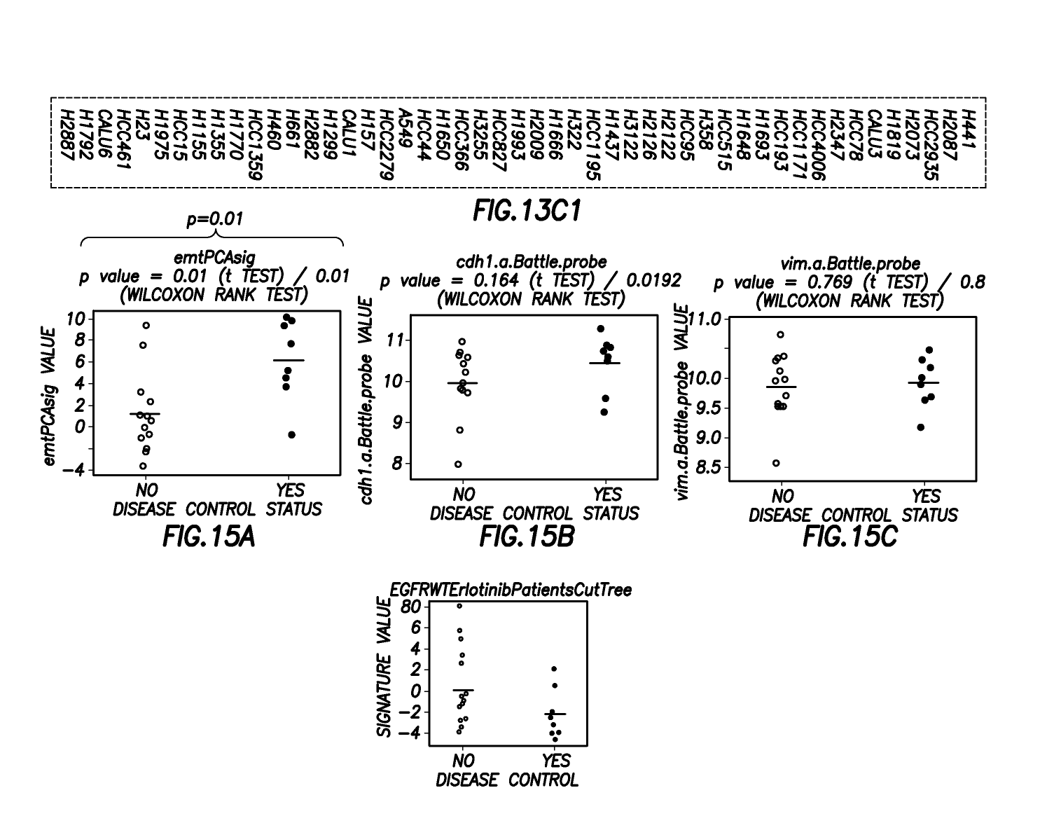

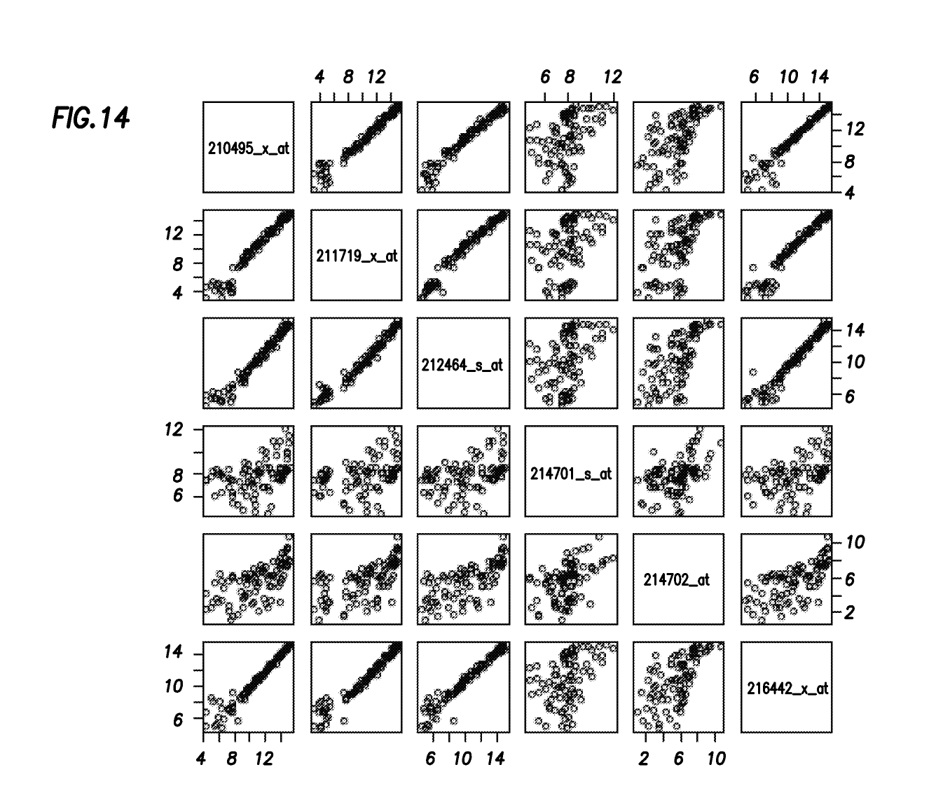

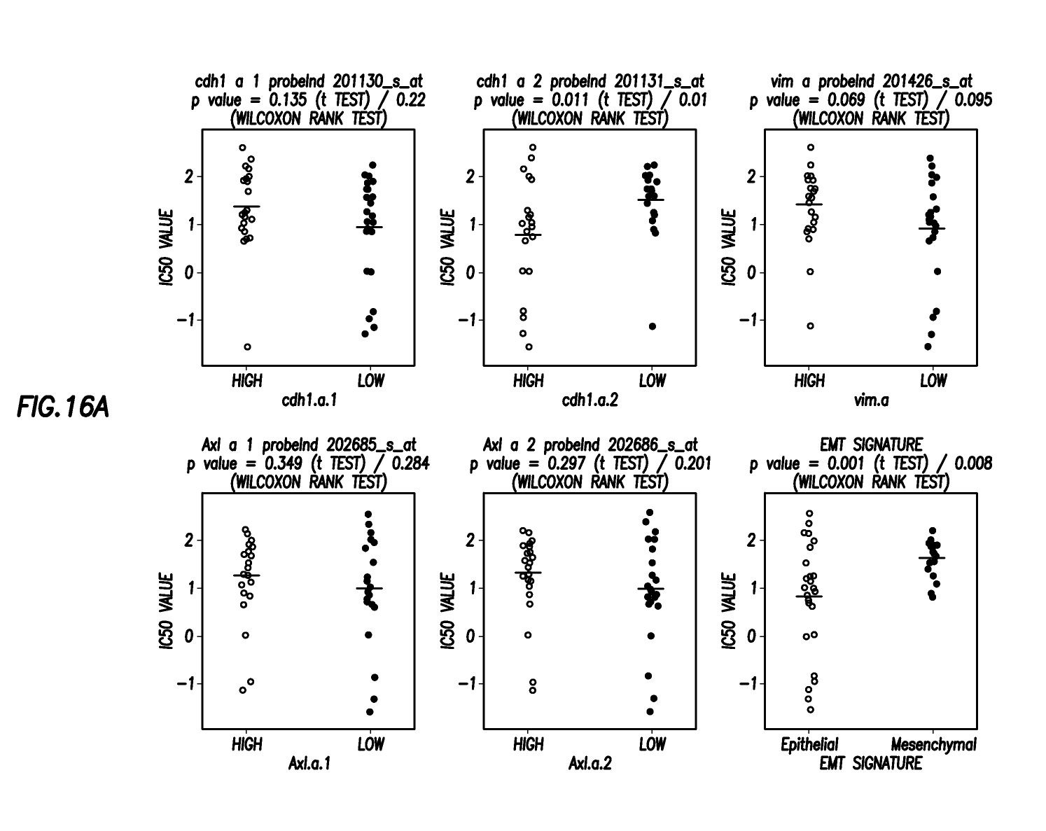

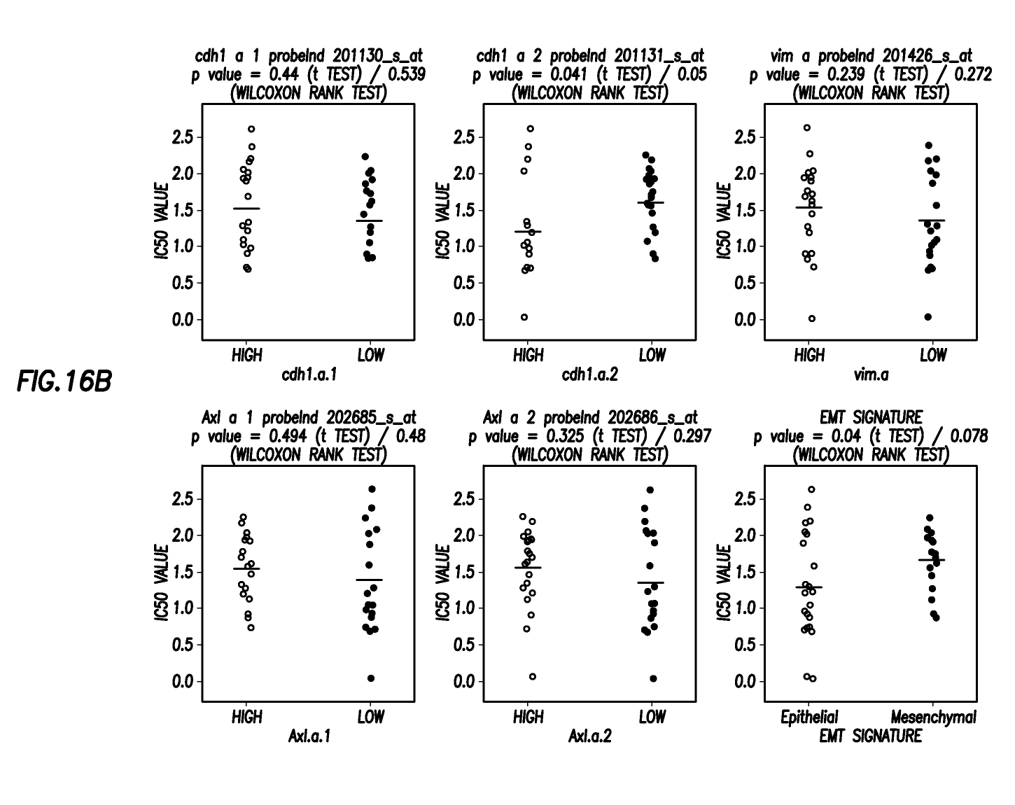

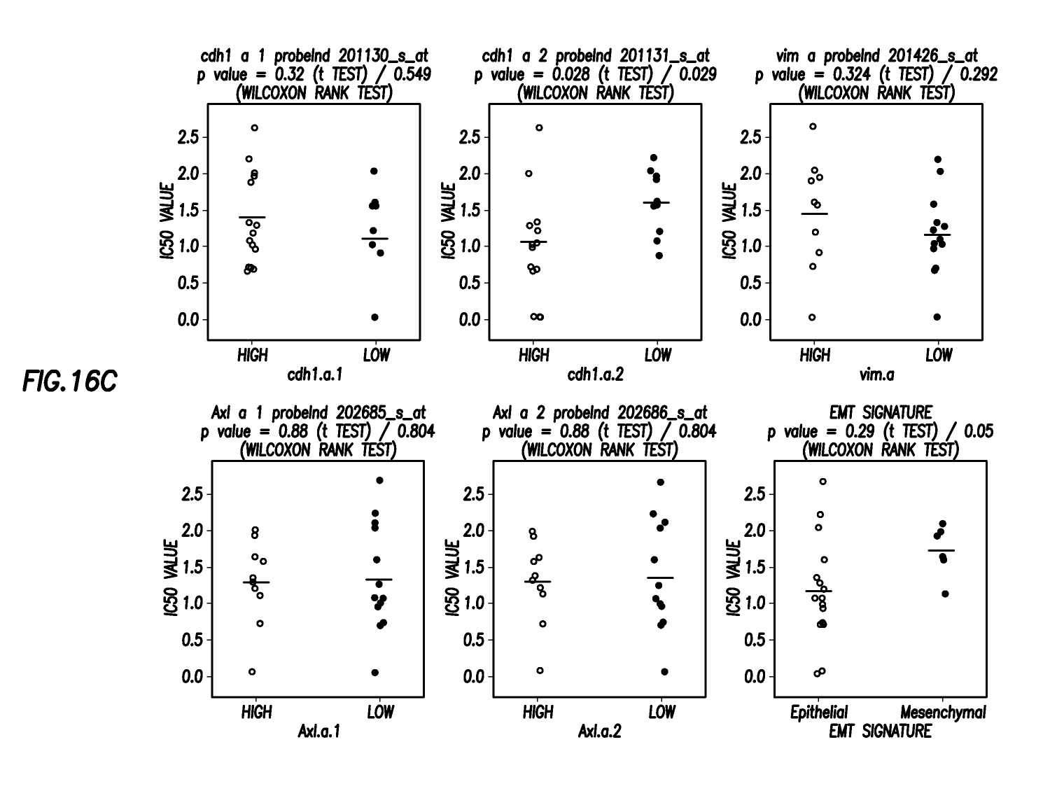

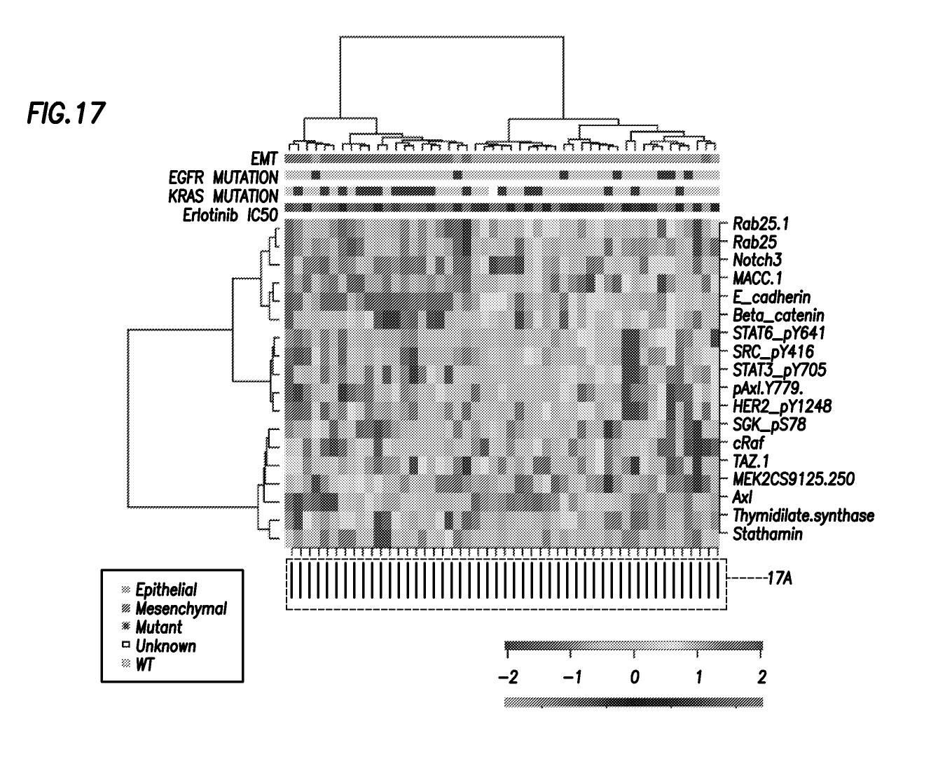

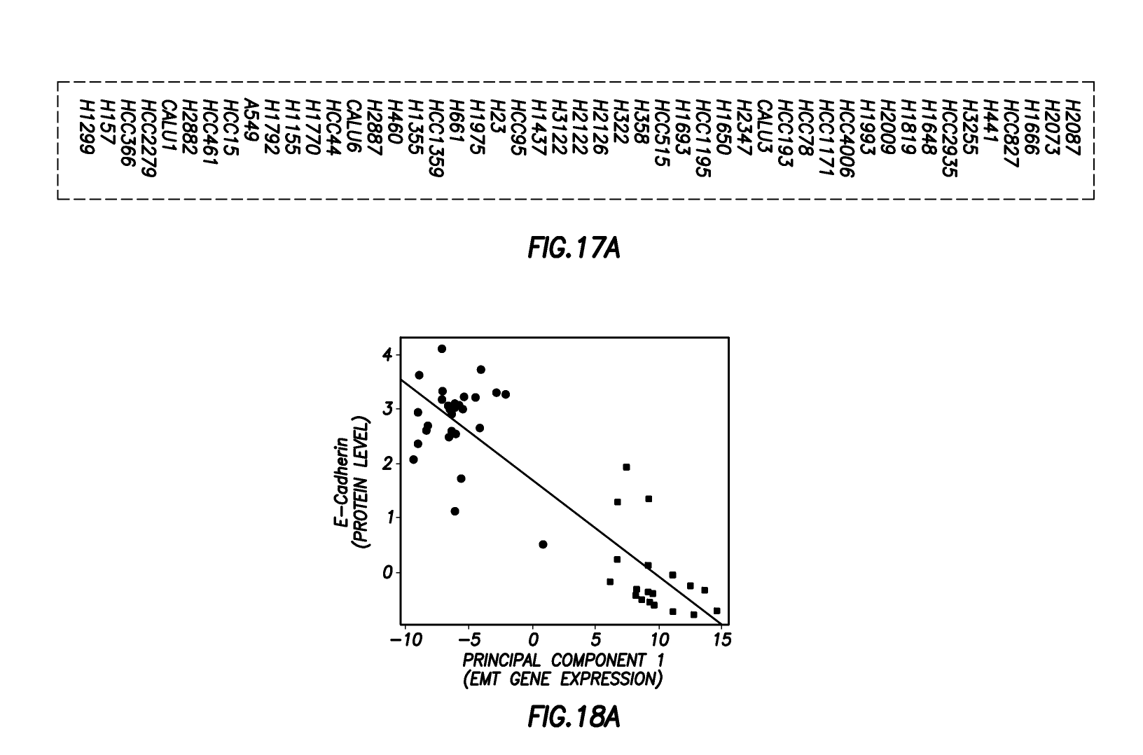

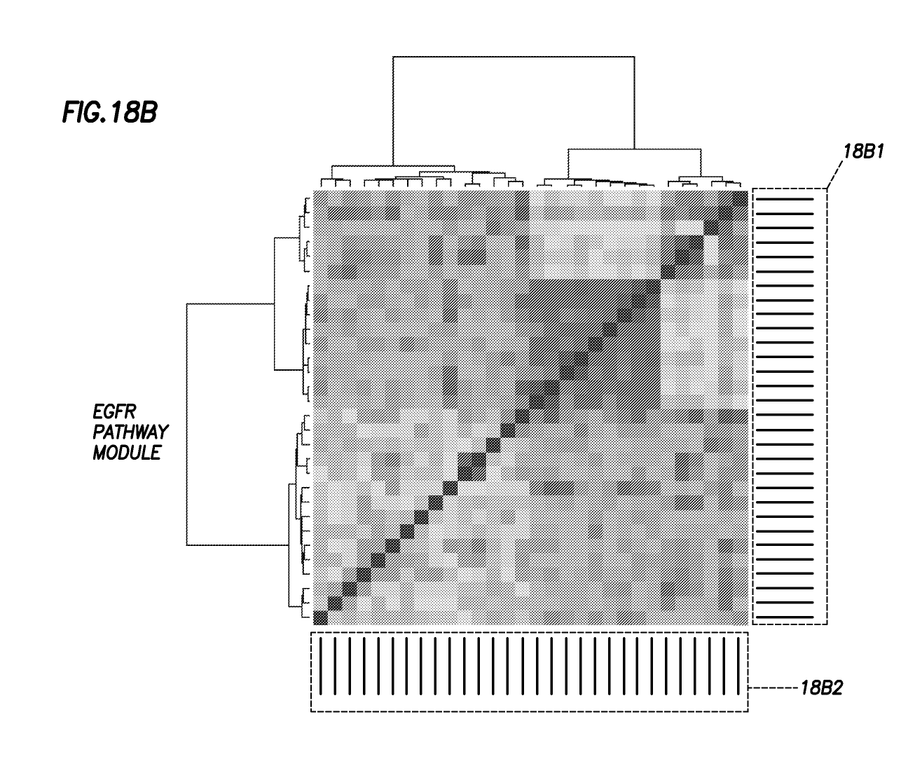

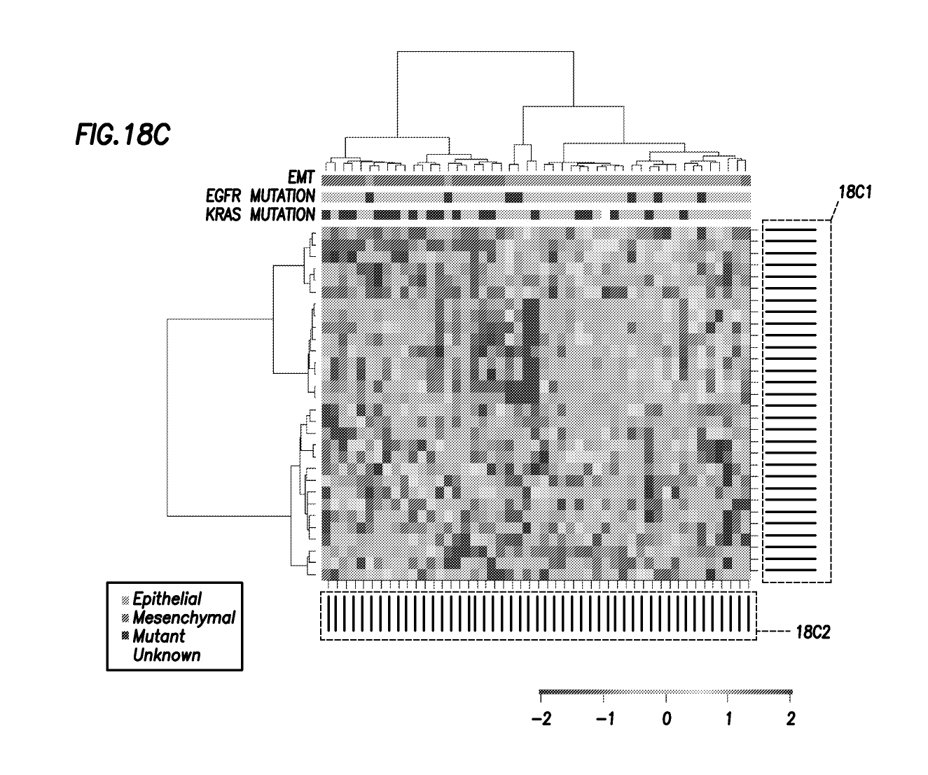

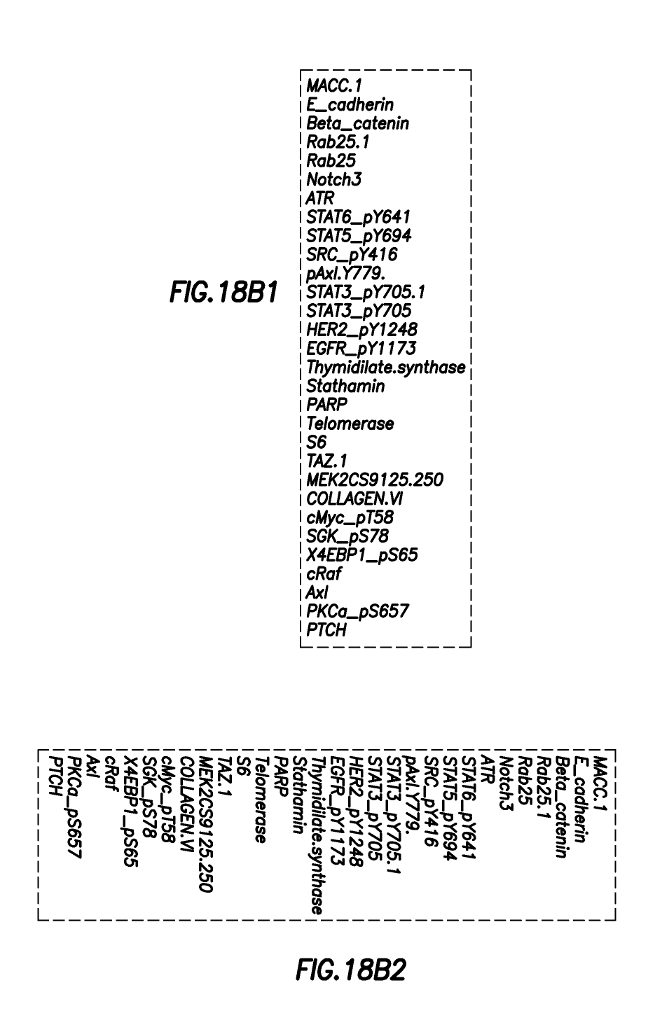

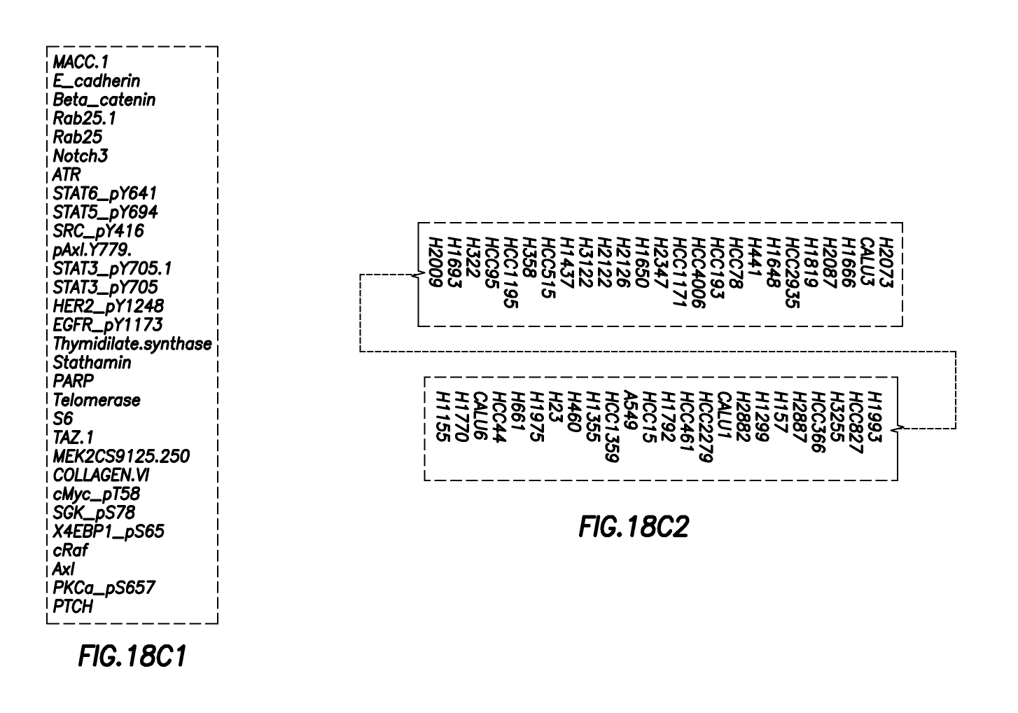

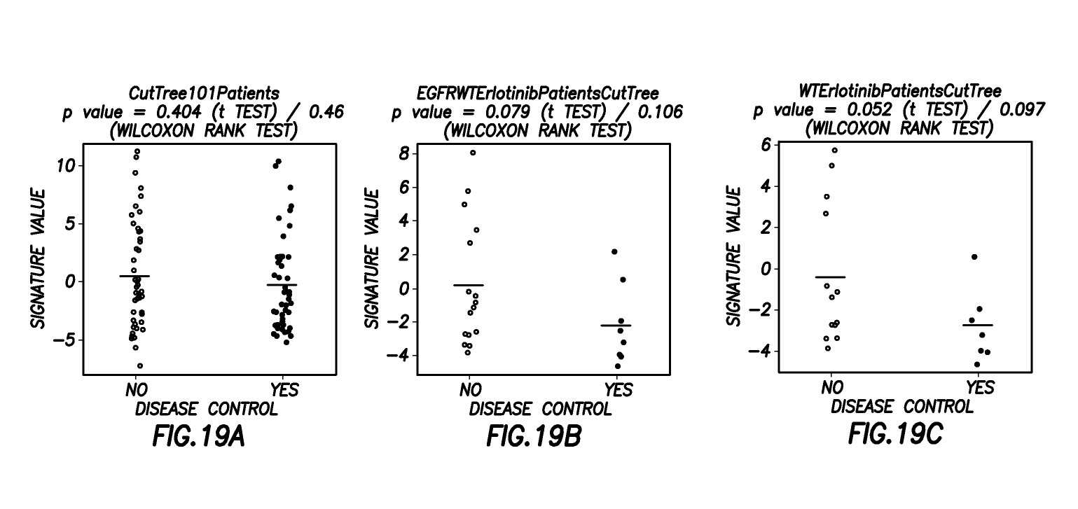

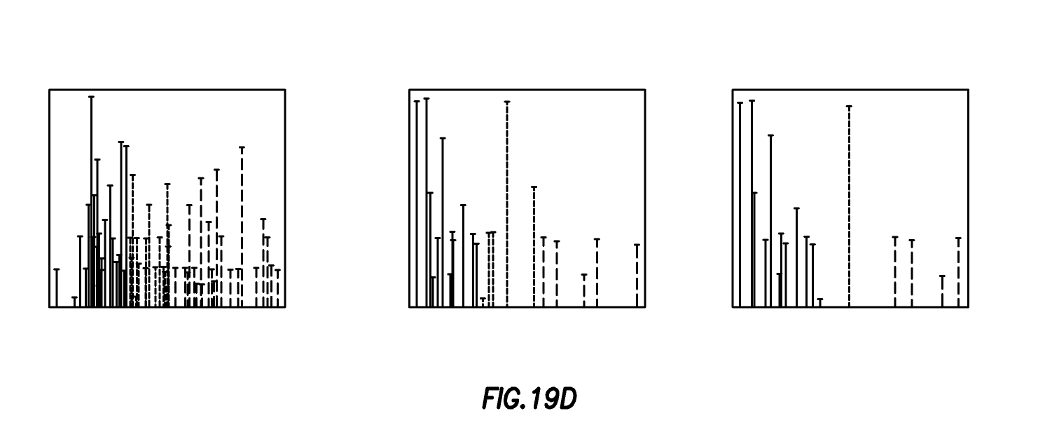

Specifically, as set out in Example 1 below, to better characterize EMT and its association with drug response in NSCLC, we performed an integrated analysis of gene expression profiling from several microarray platforms as well as high-throughput functional proteomic profiling. See generally, FIGS. 1 through 19. By cross-validating gene expression data from two independent microarray platforms in our training set of NSCLC cell lines, we derived a robust EMT gene expression signature. We also performed an integrated analysis of the EMT gene signature and high-throughput proteomic profiling of key oncogenic pathways to explore differences in signaling pathways between epithelial and mesenchymal lines. Finally, we tested the ability of the EMT signature to predict response to erlotinib and other drugs in EGFR-mutated and wild type NSCLC cell lines and patient tumor samples.

### Example I

### EMT Gene Signatures

### Materials and Methods

Cell Lines.

NSCLC cell lines were established by John D. Minna and Adi Gazdar (20, 21) or obtained through ATCC and grown in RPMI-1640 plus 10% FBS. Identities were confirmed by DNA fingerprinting.

Selection of Single Best EMT Marker Probes.

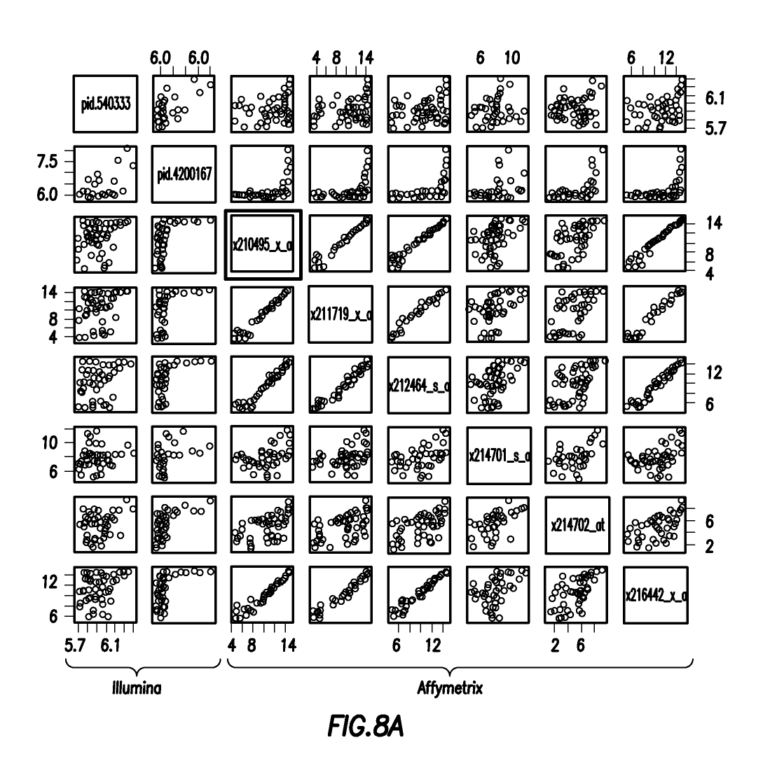

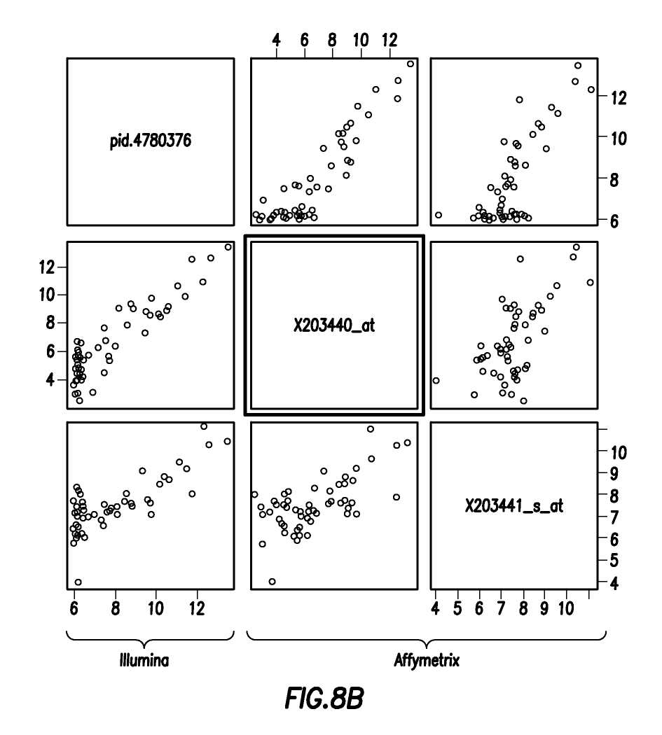

Because the NSCLC cell line panel was profiled on both Affymetrix and Illumina microarray platforms, we were able to select the single best Affymetrix probe sets for CDH1, VIM, CDH2, and FN1 on the basis of their correlations with other Affymetrix probes and Illumina WG v2 probes for the same gene transcript (FIG. 8). For example, measurements from the two Affymetrix CDH1 probes (201130_s_at and 201131_s_at) were not well correlated (r=0.303), suggesting that at least one was likely to be of poor quality. To determine which probe set most accurately assessed CDH1 mRNA, we compared measurements from the Affymetrix CDH1 probe sets with those from the Illumina WGv2 CDH1 probe set. Probe set 201131_s_at correlated best with the Illumina CDH1 set (r=0.701 versus 0.201) and, therefore, was selected to represent CDH1. Affymetrix probe set 201131_s_at also correlated well with E-cadherin protein levels (r=0.865), lending support to that method for selecting the best probes for specific markers.

For N-cadherin (CDH2), Aff 203440_at and Aff 203411_s_at were highly correlated (r=0.802). Aff 203440_at was selected for the analysis because of its better correlation with the Illumina CDH2 probe (r=0.904 versus 0.730). Fibronectin (FN1) probe set 210495_x_at was selected from among four good Affymetrix probe sets because it had the highest correlation with the Illumina FN1 probes. Although the Affymetrix arrays include only one probe set for vimentin (VIM) (201426_s_at), measurements from that set correlated well (r=0.958) with that from the Illumina WGv2 VIM probe set (III 50671). The Affymetrix probe was therefore considered to be an accurate measure of VIM transcript expression.

Once the best probes were selected, EMT signature genes were selected based on their correlation with the four EMT genes (absolute r-value ≧0.65 for CDH1 and VIM, ≧0.52 for CDH2 and FN1) and their bimodal distribution across the training set, as described in results. By limiting the EMT signature to genes expressed among the cell lines at either relatively high or low levels, but not in between, we expected to increase the likelihood that the signature could separate patient tumors into distinct epithelial and mesenchymal groups. Hierarchical clustering and Principal Component Analysis (PCA) algorithms were used on mRNA expression data to evaluate the EMT signature.

Expression Profiling of Cell Lines.

Affymetrix microarray results were previously published and archived at the Gene Expression Omnibus repository (http://www.ncbi.nlm.nih.gov/geo/, GEO accession GSE4824). Zhou B. B., et al., Targeting ADAM-Mediated Ligand Cleavage to Inhibit HERS and EGFR Pathways in Non-Small Cell Lung Cancer, Cancer Cell 10:39-50 (2006); Edgar R., et al., Gene Expression Omnibus: NCBI Gene Expression and Hybridization Array Data Repository, Nucleic Acids Res. 30:207-10 (2002); Barrett T., et al., NCBI GEO: Archive for Functional Genomics Data Sets—10 Years On, Nucleic Acids Res. 39:D1005-10. Illumina v2 (GSE32989) and v3 (GSE32036) results have been deposited in the GEO repository. Microarray data was used to derive a platform-independent, 76-gene expression signature was derived as described in Supplemental Methods.

Gene Expression Profiling of BATTLE Tumors.

BATTLE (Biomarker-integrated Approaches of Targeted Therapy for Lung Cancer Elimination) was a randomized, biomarker-based clinical trial for patients with recurrent or metastatic NSCLC in the second-line setting (Trial registration ID: NCT00409968). Kim E. S. H. R., The BATTLE Trial: Personalizing Therapy for Lung Cancer, Cancer Discovery 1:43-51 (2011). mRNA from tumors obtained via core-needle biopsy at enrollment were profiled on Human Gene 1.0 ST array, Affymetrix. Array results were deposited in the GEO repository (GSE33072).

Drug Sensitivity of Cell Lines.

For each drug, the concentration required to inhibit 50% growth (IC₅₀) was measured by MTS assay ≧3 times in NSCLC cell lines. Average values were used for analysis as described. Gandhi J., et al., Alterations in Genes of the EGFR Signaling Pathway and Their Relationship to EGFR Tyrosine Kinase Inhibitor Sensitivity in Lung Cancer Cell Lines, PLoS One 4:e4576 (2009). Axl inhibitor SGI-7079 was generated as described in Supplemental Methods. The effect of erlotinib, SGI-7079, or the combination of erlotinib and SGI-7079 on proliferation was assayed using CellTiter-Glo Luminescent Cell Viability kit (Promega), as described. Chou T. C., et al., Quantitative Analysis of Dose-Effect Relationships: The Combined Effects of Multiple Drugs or Enzyme Inhibitors. Adv Enzyme Regul. 22:27-55 (1984); Johnson F. M., et al., Abrogation of Signal Transducer and Activator of Transcription 3 Reactivation After Src Kinase Inhibition Results in Synergistic Antitumor Effects, Clin Cancer Res. 13:4233-44 (2007).

Protein Profiling by Reverse-Phase Protein Array (RPPA) and Western Blot.

RPPA studies were performed as described. Byers L. A., et al., Reciprocal Regulation of C-Src And STATS in Non-Small Cell Lung Cancer, Clin Cancer Res. 15:6852-61 (2009). Protein lysate was collected from sub-confluent cultures after 24 hours in complete medium. RPPA slides were printed from lysates. Immunostaining was performed and analyzed, as described in Supplemental Methods. Primary antibodies included pEGFR (Y1173), pSTAT3 (Y705), pSTAT5 (Y694), pSTAT6 (Y641), pSrc (Y416), and E-cadherin (Cell Signaling); pHer2 (Y1248) (Upstate Biotechnology); Axl (Abcam), and Rab25 (Covance).

Generation and Characterization of AXL Inhibitor SGI-7079.

Purified recombinant AXL kinase was used to screen a library of structures with appropriate drug-like scaffolds to identify potential inhibitors. Hits from the screen were confirmed and r analyzed by selection criteria including Lipinski rules. One pyrrolopyrimidine-based compound was selected for structure-activity relationship efforts. Optimization of this scaffold and subsequent evaluation led to the generation of compound SG1-7079 as the lead candidate inhibitor (FIG. 10). SGI-7079 exhibited a Kᵢ=5.7 nM for AXL and inhibited Gas6 ligand-induced tyrosine phosphorylation of human AXL expressed in HEK293T cells (EC₅₀=100 nM). SGI-7079 was screened against a panel of protein kinases to determine both selectivity and biochemical potency. SGI-7079 inhibited TAM family members MER and Tyro3 similarly as AXL, and showed potent, low nM inhibition of Syk, Flt1, Flt3, Jak2, TrkA, TrkB, PDGFRβ and Ret kinases.

RPPA Data Processing and Statistical Analysis.

MicroVigene software (VigeneTech, Carlisle, Mass.) and an R package developed in house were used to assess spot intensity. Protein levels were quantified by the SuperCurve method (http://bioinformatics.mdanderson.org/OOMPA) as previously described. Hu J., et al., Non-Parametric Quantification of Protein Lysate Arrays, Bioinformatics 23:1986-94 (2007); Nanjundan M., et al., Proteomic Profiling Identifies Pathways Dysregulated in Non-Small Cell Lung Cancer and an Inverse Association of AMPK and Adhesion Pathways With Recurrence, J Thorac Oncol. 5:1894-904 (2010). Data were log-transformed (base 2) and median-control normalized across all proteins within a sample. Differences in protein expression between epithelial and mesenchymal cell lines were compared by t-test. Pearson correlation between E-cadherin protein expression levels and first principal component of the EMT signature derived from mRNA expression data was then assessed. All statistical analyses were performed using R packages (version 2.10.0)

### Results

A 76-Gene EMT Signature Classifies NSCLC Cell Lines into Distinct Epithelial and Mesenchymal Groups.

Using a training set of 54 NSCLC cell lines profiled on Affymetrix U133A, U133B, and Plus2.0 arrays, we selected genes for the EMT gene expression signature based on two criteria aimed at increasing the robustness and potential applicability of the signature across different platforms. First, we identified genes whose mRNA expression levels were either positively or negatively correlated with the single best probe for at least one of four putative EMT markers—E-cadherin (CDH1), vimentin (VIM), N-cadherin (CDH2), and/or fibronectin 1 (FN1). For this analysis, the best probe to represent each of the four genes was selected based on its strong correlation with other probes for the same gene within a microarray platform and/or across platforms (see Methods). From that set, we selected only those genes whose mRNA expression followed a bimodal distribution pattern across cell lines (bimodal index >1.5). Wang J., et al., The Bimodality Index: A Criterion for Discovering and Ranking Bimodal Signatures From Cancer Gene Expression Profiling Data, Cancer Inform. 7:199-216 (2009).

TABLE 1
THE EMT SIGNATURE GENES

| | | | | | | | Bimodal | | | | |
|---|---|---|---|---|---|---|---|---|---|---|---|
| Affymetrix Probe | Gene Symbol | Gene Name | E-cadherin | Vimentin | N-cadherin | Fibronectin 1 | index | Accession | LocusLink | Chromosome | Cytoband |
| 212764_at | ZEB1 | Zinc finger E-box binding homeobox 1 | −0.78 | 0.62 | 0.38 | −0.05 | 1.75 | BX647794 | 6935 | 10 | 10p11.22 |
| 210875_s_at | ZEB1 | Zinc finger E-box binding homeobox 1 | −0.68 | 0.54 | 0.16 | −0.17 | 2.25 | NM_030751 | 6935 | 10 | 10p11.22 |
| 225793_at | LIX1L | Lix1 homolog (mouse)-like | −0.67 | 0.54 | 0.28 | −0.12 | 1.81 | AK128733 | 128077 | 1 | 1q21.1 |
| 201426_s_at | VIM | Vimentin | −0.55 | 1.00 | 0.42 | 0.30 | 1.68 | NM_003380 | 7431 | 10 | 10p12.33 |
| 202685_s_at | AXL | AXL receptor tyrosine kinase | −0.45 | 0.60 | 0.54 | 0.24 | 1.84 | NM_021913 | 558 | 19 | 19p13.2 |
| 201069_at | MMP2 | “Matrix metallopeptidase 2 (getatinase A, | −0.27 | 0.30 | 0.56 | 0.22 | 1.83 | NM_004530 | 4313 | 16 | 16q12.2 |
| 225524_at | ANTXR2 | Anthrax toxin receptor 2 | −0.25 | 0.40 | 0.09 | 0.55 | 1.74 | NM_058172 | 118429 | 4 | 4q21.21 |
| 226891_at | C3orf21 | Chromosome 3 open reading frame 21 | −0.14 | −0.14 | −0.04 | −0.54 | 2.19 | NM_152531 | 152002 | 3 | 3q29 |
| 214702_at | FN1 | Fibronectin 1 | −0.08 | 0.27 | 0.09 | 0.58 | 1.62 | NM_054034 | 2335 | 2 | 2q35 |
| 212298_at | NRP1 | Neuropilin 1 | −0.01 | 0.15 | 0.01 | 0.69 | 1.54 | NM_003873 | 8829 | 10 | 10p11.22 |
| 201506_at | TGFBI | “Transforming growth factor, beta-induce [symbol] | 0.07 | 9.09 | −0.02 | 0.58 | 1.89 | NM_000358 | 7045 | 5 | 5q31.1 |
| 229555_at | GALNT5 | UDP-N-acetyl-alpha-D-galactosamine:pol [symbol] | 0.15 | 0.22 | 0.14 | 0.55 | 1.82 | NM_014568 | 11227 | 2 | 2q24.1 |
| 208510_s_at | PPARG | Peroxisome proliferator-activated recepto [symbol] | 0.15 | 0.03 | −0.13 | 0.56 | 1.73 | NM_015869 | 5468 | 3 | 3p25.2 |
| 211719_x_at | FN1 | Fibronectin 1 | 0.15 | 0.27 | 0.11 | 0.97 | 1.50 | NM_212482 | 2335 | 2 | 2q35 |
| 211732_x_at | HNMT | Histamine N-methyltransferase | 0.24 | −0.03 | −0.02 | 0.57 | 1.99 | NM_00102407 [symbol] | 3176 | 2 | 2q22.1 |
| 204112_s_at | HNMT | Histamine N-methyltransferase | 0.33 | −0.07 | 0.02 | 0.63 | 1.55 | NM_006895 | 3176 | 2 | 2q22.1 |
| 224414_s_at | CARD6 | “Caspase recruitment domain family, mer [symbol] | 0.40 | −0.16 | 0.02 | 0.61 | 1.83 | NM_032587 | 84674 | 5 | 5p13.1 |
| 209488_s_at | RBPMS | RNA binding protein with multiple splicing | 0.41 | −0.22 | −0.20 | 0.54 | 1.94 | NM_00100871 [symbol] | 11030 | 6 | 8p12 |
| 218855_at | TNFRSF21 | “Tumor necrosis factor receptor superfan [symbol] | 0.48 | −0.22 | −0.06 | 0.56 | 1.51 | NM_014452 | 27242 | 6 | 6p12.3 |
| 228226_at | TMEM45B | Transmembrane protein 45B | 0.53 | −0.41 | −0.54 | 0.20 | 1.84 | NM_138788 | 120224 | 11 | 11q24.3 |
| 238742_x_at | | | 0.63 | −0.67 | −0.37 | −0.14 | 2.22 | | NA | | |
| 238778_at | MPP7 | “Membrane protein, palmitoylated 7 (MA [symbol] | 0.65 | −0.44 | −0.34 | 0.21 | 1.62 | AL832380 | 143098 | 10 | 10p11.23 |
| 219919_s_at | SSH3 | | 0.65 | −0.48 | −0.26 | 0.08 | 1.51 | NM_018276 | NA | 11 | 11q13.1 |
| 234970_at | MTAC201 | | 0.66 | −0.64 | −0.42 | 0.13 | 2.03 | | NA | | |
| 207847_s_at | MUC1 | “Mucin 1, cell surface associated” | 0.66 | −0.51 | −0.43 | 0.19 | 1.63 | NM_002456 | 4582 | 1 | 1q22 |
| 232164_s_at | EPPK1 | Epiplakin 1 | 0.66 | −0.47 | −0.23 | −0.08 | 1.95 | NM_031308 | 83481 | 8 | 8q24.3 |
| 225548_at | SHROOM3 | Shroom family member 3 | 0.67 | −0.36 | −0.25 | 0.24 | 1.84 | NM_020859 | 57619 | 4 | 4q21.1 |
| 220318_at | EPN3 | Epsin 3 | 0.67 | −0.70 | −0.48 | 0.08 | 2.07 | NM_017957 | 55040 | 17 | 17q21.33 |
| 205847_at | PRSS22 | “Protease, serine, 22” | 0.67 | −0.50 | −0.41 | 0.16 | 1.72 | NM_022119 | 54063 | 16 | 16p13.3 |
| 65517_at | AP1M2 | “Adaptor-related protein complex 1, mu 2 | 0.67 | −0.46 | −0.29 | 0.14 | 3.39 | NM_005498 | 10053 | 19 | 19p13.2 |
| 229842_at | | | 0.68 | −0.62 | −0.31 | 0.21 | 2.07 | AC099676 | NA | | |
| 204019_s_at | SH3YL1 | “SH3 domain containing, Ysc84-like 1 (S. [symbol] | 0.68 | −0.56 | −0.40 | 0.13 | 1.58 | NM_015677 | 26751 | 2 | 2p25.3 |
| 239853_at | KLC3 | Kinesin light chain 3 | 0.68 | −0.33 | −0.12 | 0.01 | 1.85 | NM_177417 | 147700 | 19 | 19q13.32 |
| 235986_at | | | 0.68 | −0.30 | −0.31 | 0.40 | 1.74 | AB065679 | NA | | |
| 224762_at | SERINC2 | Serine incorporator 2 | 0.69 | −0.45 | −0.24 | 0.06 | 1.63 | NM_178865 | 347735 | 1 | 1p35.2 |
| 204503_at | EVPL | Envoplakin | 0.69 | −0.47 | −0.42 | 0.22 | 1.78 | NM_001988 | 2125 | 17 | 17q25.1 |
| 202489_s_at | FXYD3 | FXYD domain containing ion transport re [symbol] | 0.69 | −0.70 | −0.33 | 0.05 | 2.47 | NM_021910 | 5349 | 19 | 19q13.11 |
| 201428_at | CLDN4 | Claudin 4 | 0.69 | −0.43 | −0.35 | 0.40 | 2.11 | NM_001305 | 1364 | 7 | 7q11.23 |
| 232609_at | CRB3 | Crumbs homolog 3 (Drosophila) | 0.69 | −0.43 | −0.35 | 0.05 | 1.68 | NM_174881 | 92359 | 19 | 19p13.3 |
| 219476_at | LRRC54 | “CDNA FLJ25280 fis, clone STM06543” | 0.69 | −0.41 | −0.22 | 0.32 | 2.18 | AK058009 | NA | 11 | 11q13.5 |
| 210058_at | MAPK13 | Mitogen-activated protein kinase 13 | 0.69 | −0.41 | −0.39 | 0.23 | 1.54 | NM_002754 | 5603 | 6 | 6p21.31 |
| 232165_at | EPPK1 | Epiplakin 1 | 0.70 | −0.51 | −0.27 | −0.11 | 2.13 | AL137725 | 83481 | 8 | 8q24.3 |
| 203397_s_at | GALNT3 | UDP-N-acetyl-alpha-D-galactosamine:pol [symbol] | 0.70 | −0.45 | −0.29 | 0.24 | 1.81 | NM_004482 | 2591 | 2 | 2q24.3 |
| 235144_at | | “CDNA FLJ32320 fis, clone PROST2003 [symbol] | 0.70 | −0.49 | −0.40 | 0.28 | 1.97 | AK056882 | NA | 9 | 9q21.32 |
| 221610_s_at | STAP2 | Signal transducing adaptor family membe [symbol] | 0.70 | −0.49 | −0.25 | 0.18 | 1.57 | NM_00101384 | 55620 | 19 | 19p13.3 |
| 218261_at | AP1M2 | “Adaptor-related protein complex 1, mu 2 | 0.70 | −0.45 | −0.16 | 0.14 | 2.71 | NM_005498 | 10053 | 19 | 19p13.2 |
| 200606_at | DSP | Desmoplakin | 0.70 | −0.56 | −0.30 | −0.09 | 1.57 | NM_004415 | 1832 | 6 | 6p24.3 |
| 219411_at | ELMO3 | Engulitment and cell mocility 3 | 0.71 | −0.52 | −0.41 | 0.09 | 1.71 | NM_024712 | 79767 | 16 | 16q22.1 |
| 235148_at | KRTCAP3 | Keratinocyte associated protein 3 | 0.71 | −0.59 | −0.42 | 0.02 | 2.50 | NM_173853 | 200634 | 2 | 2p23.3 |
| 224650_at | MAL2 | “Mal, T-cell differentiation protein 2” | 0.71 | −0.50 | −0.44 | 0.21 | 2.55 | NM_052886 | 114569 | 8 | 8q24.12 |
| 224097_s_at | F11R | | 0.72 | −0.45 | −0.38 | 0.09 | 1.57 | NM_144504 | NA | 1 | 1q23.3 |
| 238689_at | GPR110 | G protein-coupled receptor 110 | 0.72 | −0.38 | −0.41 | 0.32 | 1.79 | NM_153840 | 266977 | 6 | 5p12.3 |
| 228441_s_at | | | 0.72 | −0.38 | −0.29 | 0.12 | 1.64 | AC092611 | NA | | |
| 212070_at | GPR56 | G protein-coupled receptor 56 | 0.72 | −0.53 | −0.33 | 0.27 | 1.80 | NM_201525 | 9289 | 16 | 16q13 |
| 201650_at | KRT19 | Keratin 19 | 0.73 | −0.43 | −0.33 | 0.35 | 2.58 | NM_002276 | 3880 | 17 | 17q21.2 |
| 222830_at | GRHL1 | Grainyhead-like 1 (Drosophila) | 0.73 | −0.52 | −0.45 | 0.09 | 1.85 | NM_198182 | 29841 | 2 | 2p25.1 |
| 218792_s_at | BSPRY | B-box and SPRY domain containing | 0.73 | −0.51 | −0.37 | 0.09 | 1.53 | NM_017683 | 54836 | 9 | 9q32 |
| 228865_at | C1orf116 | Chromosome 1 open reading frame 116 | 0.73 | −0.30 | −0.10 | 0.44 | 1.58 | NM_023938 | 79098 | 1 | 1q32.1 |
| 218677_at | S100A14 | S100 calcium binding protein A14 | 0.73 | −0.65 | −0.37 | 0.09 | 1.96 | NM_020672 | 57402 | 1 | 1q21.3 |
| 210715_s_at | SPINT2 | “Serine peptidase inhibitor, Kunitz type, 2 | 0.73 | −0.44 | −0.35 | 0.07 | 1.91 | NM_021102 | 10653 | 19 | 19q13.2 |
| 236489_at | | | 0.74 | −0.38 | −0.28 | 0.32 | 1.89 | AB065679 | NA | | |
| 238439_at | ANKRD22 | Ankyrin repeat domain 22 | 0.74 | −0.49 | −0.48 | 0.17 | 1.76 | NM_144590 | 118932 | 10 | 10q23.31 |
| 216905_s_at | ST14 | Suppression of tumorigenicity 14 (colon [symbol] | 0.74 | −0.50 | −0.35 | 0.15 | 2.09 | NM_021978 | 6768 | 11 | 11q24.3 |
| 219388_at | GRHL2 | Grainyhead-like 2 (Drosophila) | 0.74 | −0.59 | −0.44 | 0.11 | 1.85 | NM_024915 | 79977 | 8 | 8q22.3 |
| 205980_s_at | PRR5 | Rho GTPase activating protein 8 | 0.74 | −0.46 | −0.32 | 0.00 | 2.98 | NM_00101752 [symbol] | 55615 | 22 | 22q13.31 |
| 222746_s_at | BSPRY | B-box and SPRY domain containing | 0.75 | −0.44 | −0.39 | 0.18 | 1.77 | NM_017689 | 54836 | 9 | 9q32 |
| 35148_at | TJP3 | Tight junction protein 3 (zona occldens [symbol] | 0.75 | −0.61 | −0.38 | 0.05 | 1.74 | NM_014428 | 27134 | 19 | 19p13.3 |
| 202286_s_at | TACSTD2 | Tumor-associated calcium signal transdu [symbol] | 0.75 | −0.49 | −0.30 | 0.18 | 2.15 | NM_002353 | 4070 | 1 | 1p32.1 |
| 203256_at | CDH3 | “Cadherin 3, type 1, P-cadherin (placenta | 0.75 | −0.42 | −0.31 | 0.22 | 2.40 | NM_001793 | 1001 | 16 | 16q22.1 |
| 236058_at | C1orf172 | Chromosome 1 open reading frame 172 | 0.76 | −0.64 | −0.40 | 0.18 | 2.39 | NM_152365 | 125895 | 1 | 1p36.11 |
| 205709_s_at | CDS1 | CDP-diacylglycerol synthase (phosphatid [symbol] | 0.76 | −0.50 | −0.49 | 0.16 | 1.56 | NM_001263 | 1040 | 4 | 4q12.23 |
| 37117_at | PRR5 | Rho GTPase activating protein 8 | 0.76 | −0.48 | −0.32 | 0.01 | 1.77 | NM_00101752 [symbol] | 55615 | 22 | 22q13.31 |
| 203780_at | MPZL2 | Myelin protein zero-like 2 | 0.76 | −0.50 | −0.38 | 0.13 | 1.69 | NM_005797 | 10205 | 11 | 11q23.3 |
| 223681_s_at | INADL | | 0.76 | −0.57 | −0.15 | 0.09 | 1.59 | AB044807 | NA | 1 | 1p31.3 |
| 223895_s_at | EPN3 | Epsin 3 | 0.76 | −0.65 | −0.40 | 0.13 | 2.20 | NM_017957 | 55040 | 17 | 17q21.33 |
| 219121_s_at | RBM35A | RNA binding motif protein 35A | 0.76 | −0.54 | −0.38 | 0.22 | 2.12 | NM_017697 | 54845 | 8 | 8q22.1 |
| 226403_at | TMC4 | Transmembrane channel-like 4 | 0.77 | −0.56 | −0.38 | 0.18 | 1.53 | NM_144686 | 147798 | 19 | 19q13.42 |
| 226535_at | ITGB6 | “integrin, beta 6” | 0.77 | −0.45 | −0.37 | 0.36 | 2.25 | AK026736 | 3894 | 2 | 2q24.2 |
| 225822_at | TMEM125 | Transmembrane protein 125 | 0.78 | −0.55 | −0.40 | 0.18 | 2.33 | NM_144626 | 128218 | 1 | 1p34.2 |
| 205977_s_at | EPHA1 | EPH receptor A1 | 0.78 | −0.54 | −0.44 | 0.24 | 2.05 | NM_005232 | 2041 | 7 | 7q34 |
| 226185_at | CDS1 | “CDNA: FLJ23044 fis, clone LNG02454” | 0.78 | −0.59 | −0.37 | 0.24 | 2.25 | AK025697 | NA | 4 | 4p21.23 |
| 227803_at | ENPP5 | Ectonucleotide pyrophosphatase/phosph [symbol] | 0.79 | −0.45 | −0.21 | 0.23 | 1.88 | NM_021572 | 59084 | 6 | 6p12.3 |
| 202005_at | ST14 | Suppression of tumorigenicity 14 (colon [symbol] | 0.79 | −0.51 | −0.36 | 0.17 | 2.34 | NM_021978 | 6768 | 11 | 11q24.3 |
| 229292_at | EPB41L5 | Erythrocyte membrane protein band 4.1 li [symbol] | 0.79 | −0.55 | −0.49 | −0.05 | 1.92 | BC032822 | 57669 | 2 | 2q14.2 |
| 202454_s_at | ERBB3 | V-erb-b2 erythroblastic leukemia viral on [symbol] | 0.79 | −0.53 | −0.34 | 0.15 | 1.64 | NM_001982 | 2065 | 12 | 12q13.2 |
| 218185_at | RAB25 | “RAB25, member RAS oncogene family” | 0.80 | −0.50 | −0.34 | 0.21 | 2.88 | NM_020387 | 57111 | 1 | 1q22 |
| 202525_at | PRSS8 | “Protease, serine 8” | 0.80 | −0.58 | −0.37 | 0.19 | 2.01 | NM_002773 | 5652 | 16 | 16p11.2 |
| 239148_at | | | 0.80 | −0.63 | −0.39 | −0.02 | 1.99 | AC009097 | NA | | |
| 213285_at | TMEM30B | Transmembrane protein 30B | 0.80 | −0.60 | −0.40 | 0.16 | 2.03 | NM_00101797 [symbol] | 161291 | 14 | 14q23.1 |
| 242354_at | | | 0.80 | −0.73 | −0.46 | −0.04 | 2.17 | | NA | | |
| 202790_at | CLDN7 | Claudin 7 | 0.80 | −0.51 | −0.35 | 0.19 | 2.12 | NM_001307 | 1368 | 17 | 17p13.1 |
| 225846_at | RBM35A | RNA binding motif protein 35A | 0.81 | −0.63 | −0.52 | 0.06 | 2.60 | NM_00103491 [symbol] | 54845 | 8 | 8q22.1 |
| 236279_at | | | 0.81 | −0.55 | −0.27 | 0.15 | 2.37 | AC010503 | NA | | |
| 201839_s_at | TACSTD1 | Tumor-associated calcium signal transdu [symbol] | 0.82 | −0.58 | −0.39 | −0.02 | 2.49 | NM_002354 | 4072 | 2 | 2p21 |
| 226187_at | CDS1 | “CDNA: FLJ23044 fis, clone LNG02454” | 0.82 | −0.57 | −0.37 | 0.21 | 1.58 | AK026697 | NA | 4 | 4q21.23 |
| 203453_at | SCNN1A | “Sodium channel, nonvoltage-gated 1 alp [symbol] | 0.83 | −0.52 | −0.28 | 0.28 | 1.97 | NM_001038 | 6337 | 12 | 12p13.31 |
| 201131_s_at | CDH1 | | 1.00 | −0.55 | −0.22 | 0.15 | 2.44 | NM_004360 | NA | 16 | 16q22.1 |

[symbol]

indicates data missing or illegible when filed

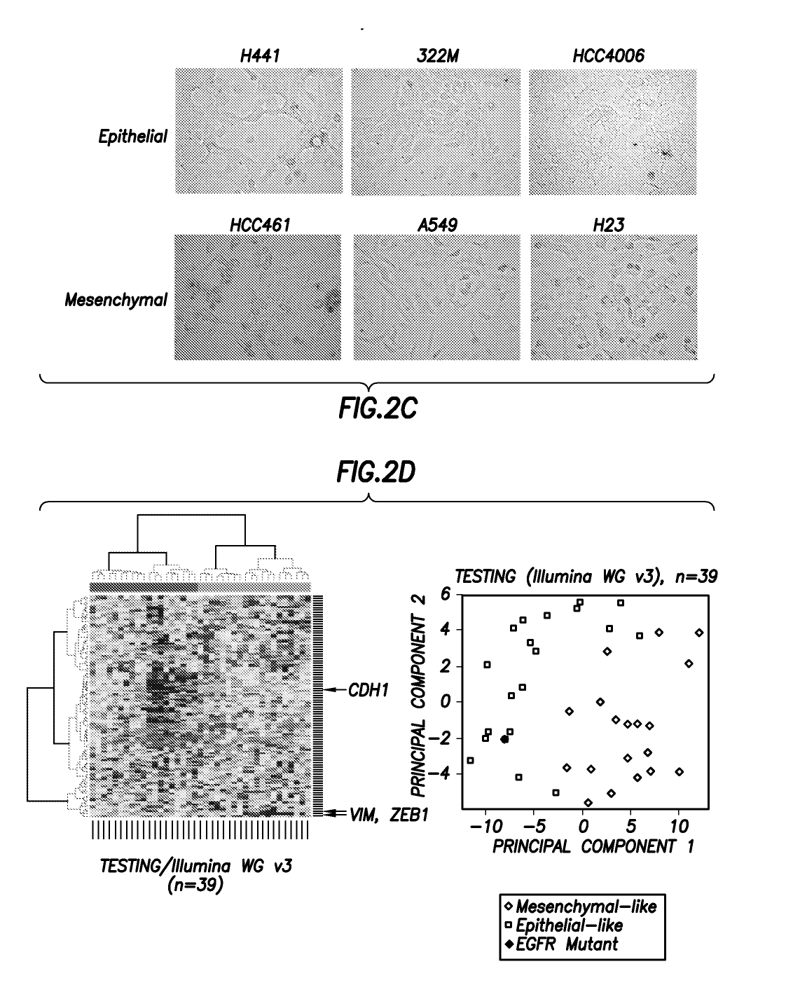

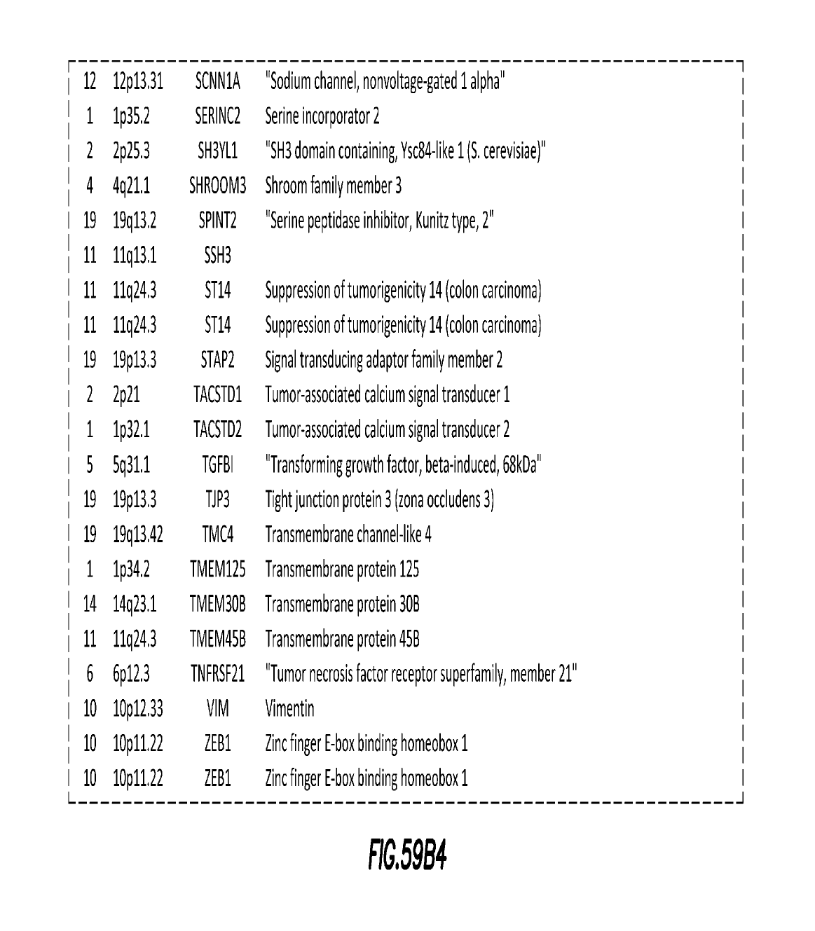

Table 1 provided immediately below lists the ninety-six probes representing 76 unique bimodally distributed genes that correlated with E-cadherin (CDH1), vimentin (VIM), N-cadherin (CDH2), and/or fibronectin 1 (FN1) were identified in the NSCLC training set. Individual probes are ranked in the table by their correlation with E-cadherin. These probes and the associated information are also provided in FIG. 59. Note that CDH2 itself did not meet the criterion for bimodal distribution so it was not included in the gene signature. Also, the NSCLC training set clustered into distinct epithelial (n=34/54 cell lines) and mesenchymal (n=20/54) groups based on expression of signature genes (FIGS. 1 and 2B).

Specifically, as shown in FIG. 1 and identified in FIG. 59, Affymetrix probes corresponding to the EMT signature genes were clustered by two-way hierarchical clustering using Pearson correlation distance between genes (rows), Euclidean distance between cell lines (columns), and the Ward's linkage rule. NSCLC cell lines separated into distinct epithelial (green bar) and mesenchymal (FIG. 1 red bar) groups at the first major branching of the dendrogram. Mutation status for EGFR and KRAS are indicated by the color bars above the heatmap (dark blue=mutated, light blue=wild-type, white=unknown). EGFR mutations were seen only in the epithelial group. KRAS mutations were more common in the mesenchymal group and expressed higher levels of FN1 and FN1-associated genes.

FIGS. 2A and 2B show cell line classifications were concordant across platforms, with the exception of H1395 which switched from epithelial to mesenchymal group when arrayed on the Illumina WG v2 platform. The red/green color bars indicate the original E- and M-classifications based on the Affymetrix data. First principal component analysis shows good separation of the epithelial and mesenchymal groups on both Affymetrix and Illumina platforms. (C) Characteristic differences in morphology are seen between lines characterized as epithelial or mesenchymal by the EMT signature. (D) In an independent set of 39 NSCLC cell lines profiled on a third platform (Illumina WGv3), the EMT signature separated cell lines into distinct epithelial (green) and mesenchymal (red) groups by hierarchical clustering and principal component analysis. Among these cell lines, only one contained a known EGFR mutation (HCC4011) and it was classified as epithelia.

Cell lines in the mesenchymal group expressed higher levels of genes activated by EMT transcription factors ZEB1/2 and/or SNAIL1/2, including matrix metalloprotease-2 (MMP-2), vimentin, and ZEB1 itself (a target of SNAIL). Miyoshi A., et al., Snail And SIP1 Increase Cancer Invasion by Upregulating MMP Family in Hepatocellular Carcinoma Cells, Br J Cancer 90:1265-73 (2004); Yokoyama K., et al., Increased Invasion and Matrix Metalloproteinase-2 Expression by Snail-Induced Mesenchymal Transition in Squamous Cell Carcinomas, Int J Oncol. 22:891-8 (2003); Cano A., et al., The Transcription Factor Snail Controls Epithelial-Mesenchymal Transitions by Repressing E-Cadherin Expression, Nat Cell Biol. 2:76-83 (2002); Eger A., et al., Deltaefl is a Transcriptional Repressor of E-Cadherin and Regulates Epithelial Plasticity in Breast Cancer Cells, Oncogene 24:2375-85 (2005); Bindels S., et al., Regulation of Vimentin by SIP1 in Human Epithelial Breast Tumor Cells, Oncogene 25:4975-85 (2006); Guaita S., et al., Snail Induction of Epithelial to Mesenchymal Transition in Tumor Cells is Accompanied by MUC1 Repression and ZEB1 Expression, J Biol Chem. 277:39209-16 (2002). AXL, a receptor tyrosine kinase associated with EMT in breast and pancreatic cancer was also highly expressed in mesenchymal NSCLC cells. Gjerdrum C., et al., Axl is an Essential Epithelial-To-Mesenchymal Transition-Induced Regulator of Breast Cancer Metastasis and Patient Survival, Proc Natl Acad Sci USA 107:1124-9 (2010); Vuoriluoto K., et al., Vimentin Regulates EMT Induction by Slug and Oncogenic H-Ras and Migration by Governing Axl Expression in Breast Cancer, Oncogene 30:1436-48 (2011); Koorstra J. B., et al,. The Axl Receptor Tyrosine Kinase Confers an Adverse Prognostic Influence in Pancreatic Cancer and Represents a New Therapeutic Target, Cancer Biol Ther. 8:618-26 (2009).

In contrast, epithelial lines had higher expression of genes repressed by ZEB1 and SNAIL, such as CDH1, RAB25, MUC1, and claudins 4 (CLDN4) and 7 (CLDN7). Cano A., et al., The Transcription Factor Snail Controls Epithelial-Mesenchymal Transitions by Repressing E-Cadherin Expression, Nat Cell Biol. 2:76-83 (2002); Eger A., et al., Deltaefl is a Transcriptional Repressor of E-Cadherin and Regulates Epithelial Plasticity in Breast Cancer Cells, Oncogene 24:2375-85 (2005); Guaita S., et al., Snail Induction of Epithelial to Mesenchymal Transition in Tumor Cells is Accompanied by MUC1 Repression and ZEB1 Expression, J Biol Chem. 277:39209-16 (2002); Battle E., et al., The Transcription Factor Snail is a Repressor of E-Cadherin Gene Expression in Epithelial Tumour Cells, Nat Cell Biol. 2:84-9 (2000); De Craene B., et al., The Transcription Factor Snail Induces Tumor Cell Invasion Through Modulation of the Epithelial Cell Differentiation Program, Cancer Res. 65:6237-44 (2005); Ikenouchi J., et al., Regulation of Tight Junctions During the Epithelium-Mesenchyme Transition: Direct Repression of the Gene Expression of Claudins/Occludin by Snail, J Cell Sci. 116:1959-67 (2003).

The EGFR family member ERBB3 and SPINT2, a regulator of HGF, were also expressed at higher levels in epithelial lines. RAB25, a trafficking protein involved with EGFR recycling, was also strongly correlated with CDH1 expression (r=0.8) and had a high bimodal index (BI=2.88, top 3% of signature genes). Although Rab25 suppression has been described as a marker of EMT in breast cancer, this is the first time to our knowledge that it has been associated with an epithelial (versus mesenchymal) phenotype in NSCLC. Vuoriluoto K., et al., Vimentin Regulates EMT Induction by Slug and Oncogenic H-Ras and Migration by Governing Axl Expression in Breast Cancer, Oncogene 30:1436-48 (2011). As expected, all EGFR-mutant cell lines were classified by the EMT signature as epithelial, including H1975 and H820, which carry the resistance mutation T790M (FIG. 1). In contrast, KRAS mutations were more common in mesenchymal (n=12/20), as compared with the epithelial lines (n=6/34) (p=0.014 by Fischer's exact test) (FIG. 1).

### Validation on Alternate Array Platforms and in an Independent Testing Set.

Because a major goal of this study was to develop a platform-independent signature, we tested performance of the EMT signature on the Illumina WGv2 microarray platform. As with the Affymetrix platform, distinct differences were observed in the expression of Illumina probes corresponding to the 76 EMT signature genes, as reflected by hierarchical clustering and first principal component analysis (FIGS. 2A & 2B). Strikingly, classification as epithelial or mesenchymal agreed across the two platforms for 51 of the 52 cell lines tested (FIGS. 2A & 2B). We then tested the signature in 39 independent NSCLC cell lines profiled on a third platform (Illumina WG v3). As with the training set, the EMT signature separated the testing set into distinct epithelial and mesenchymal groups by hierarchical clustering and principal component analysis (FIG. 2D).

### Integrated Proteomic Analysis.

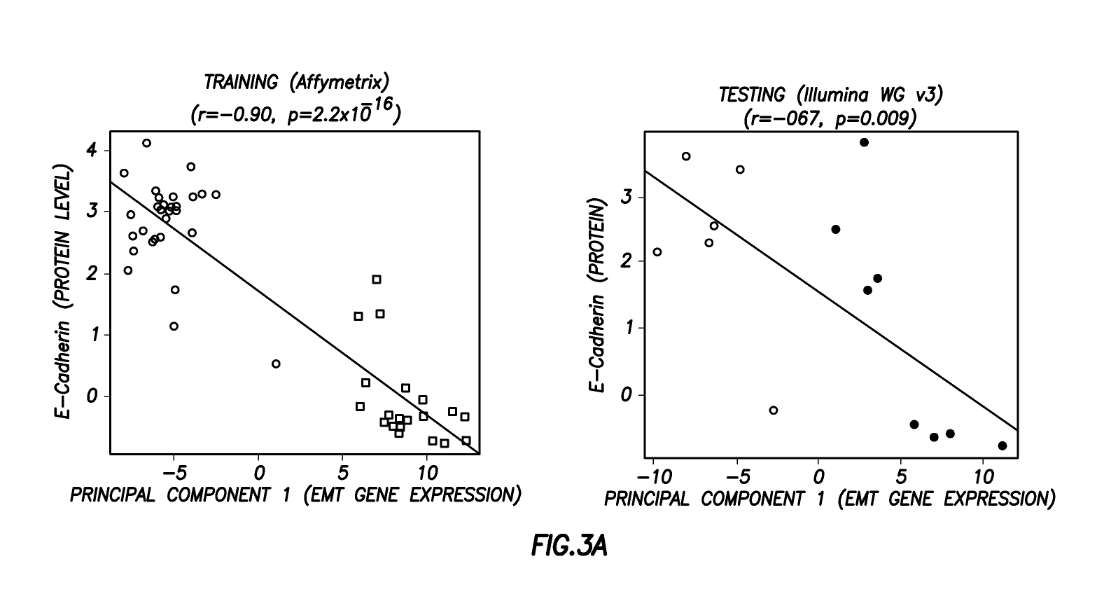

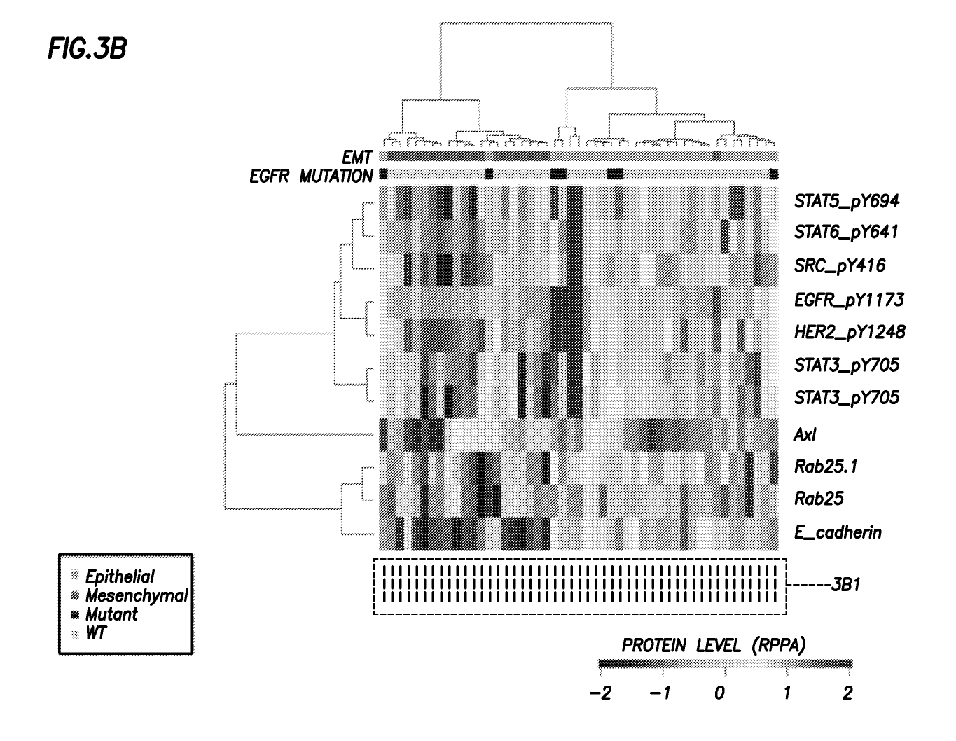

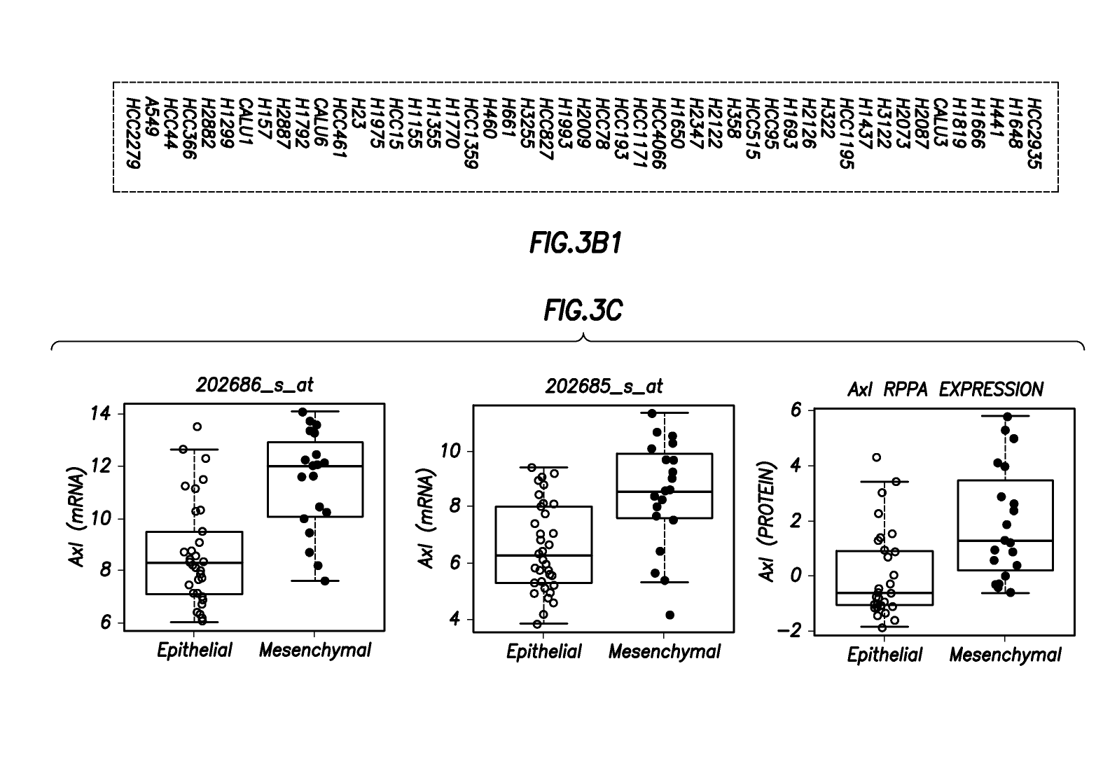

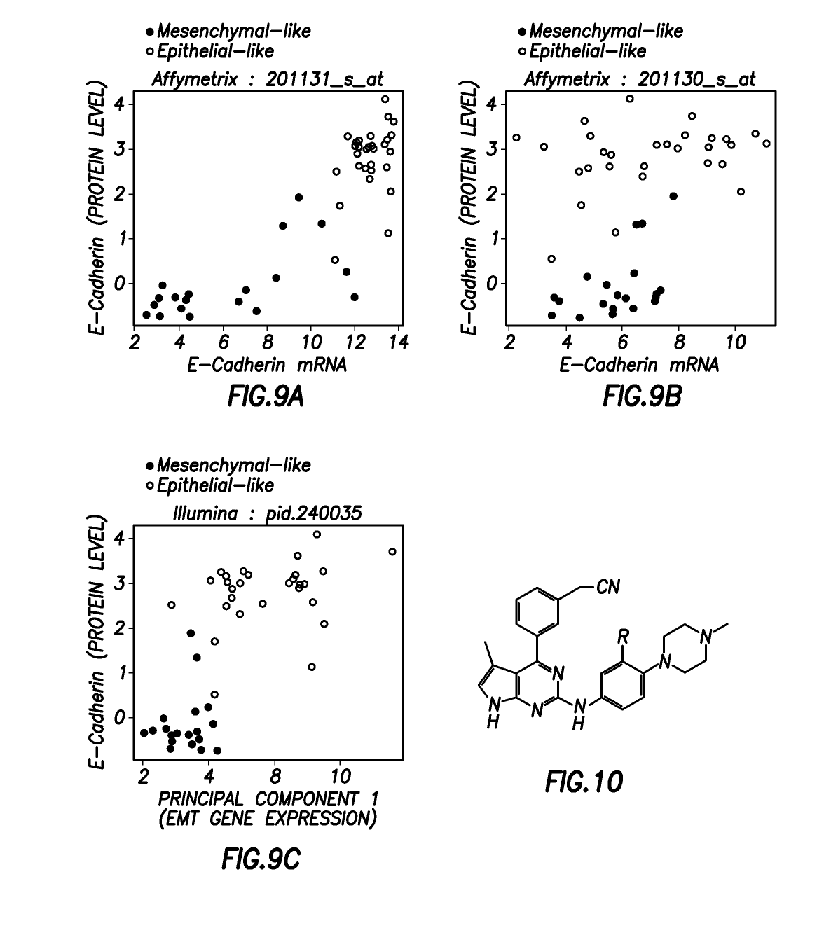

Next, we performed an integrated proteomic analysis to identify major differences in protein expression between epithelial and mesenchymal cells. Not surprisingly, out of more than 200 proteins and phosphoproteins assayed, E-cadherin differed the most between the groups (p<0.0001 by t-test) with mean E-cadherin levels 7.42-fold higher in epithelial lines, compared to mesenchymal. (FIGS. 3A & 3B). The EMT first principal component was also highly correlated with E-cadherin protein expression in the training and testing tests (p<0.01) (FIG. 3A, 3B). In contrast, correlation of E-cadherin protein with any single CDH1 mRNA probe was highly variable (r=0.37-0.86), supporting the rationale for using a signature rather than any single gene to assess EMT from mRNA expression data. (FIG. 9). Other proteins expressed at higher levels in epithelial cells included phosphorylated proteins in the EGFR pathway (e.g., pEGFR and pHER2 and downstream targets pSrc and pSTAT3, 5, and 6) (p<0.006) (FIG. 3B). Expression of two signature genes associated with EMT in other cancers, RAB25 and AXL, were also confirmed at the protein level. Consistent with the mRNA data, Rab25 protein was 1.5-fold higher in epithelial cells (p<0.0001) and positively correlated with E-cadherin protein levels (r=0.67), while Axl was 3.5-fold higher in mesenchymal lines (p=0.001).

FIG. 3 shows the results from the integrated analysis of protein expression and the EMT signature. Specifically, FIG. 3A shows E-cadherin protein levels quantified by RPPA were strongly correlated with the EMT signature first principal component in the training and testing cell line sets. FIG. 3B shows the hierarchical clustering of proteins strongly associated with an epithelial or mesenchymal signature showed higher expression of EGFR pathway proteins and Rab25 in epithelial lines. FIG. 3C shows Axl expression was significantly higher in a subset of mesenchymal cell lines at the mRNA and protein levels.

### The EMT Gene Signature Predicts Resistance to EGFR and PI3K Inhibitors In Vitro.

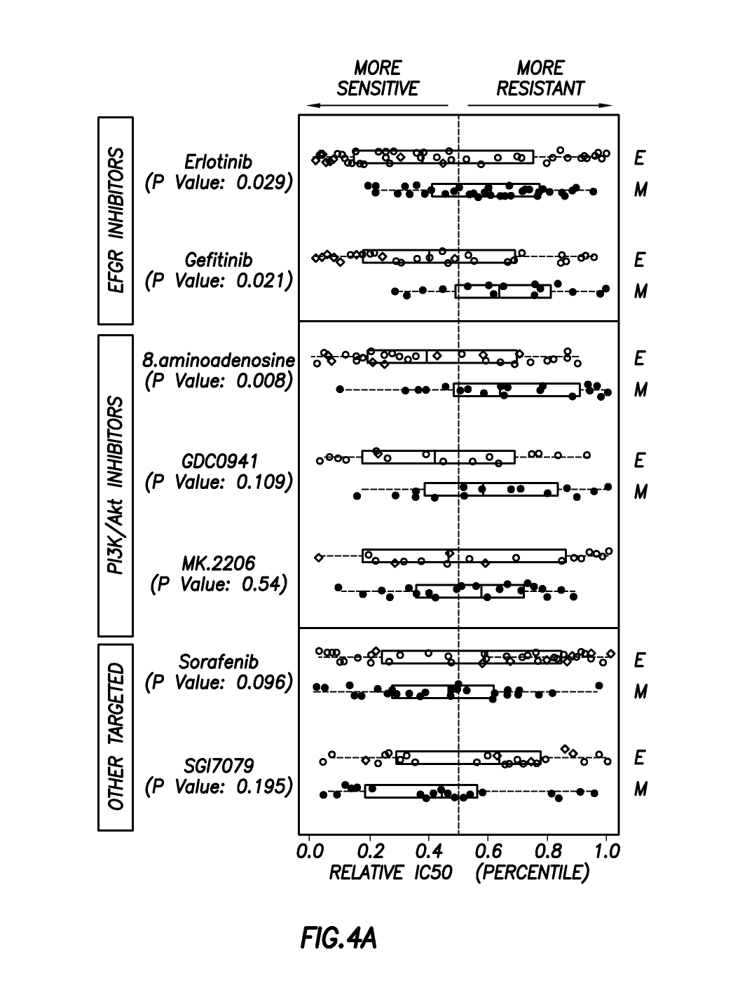

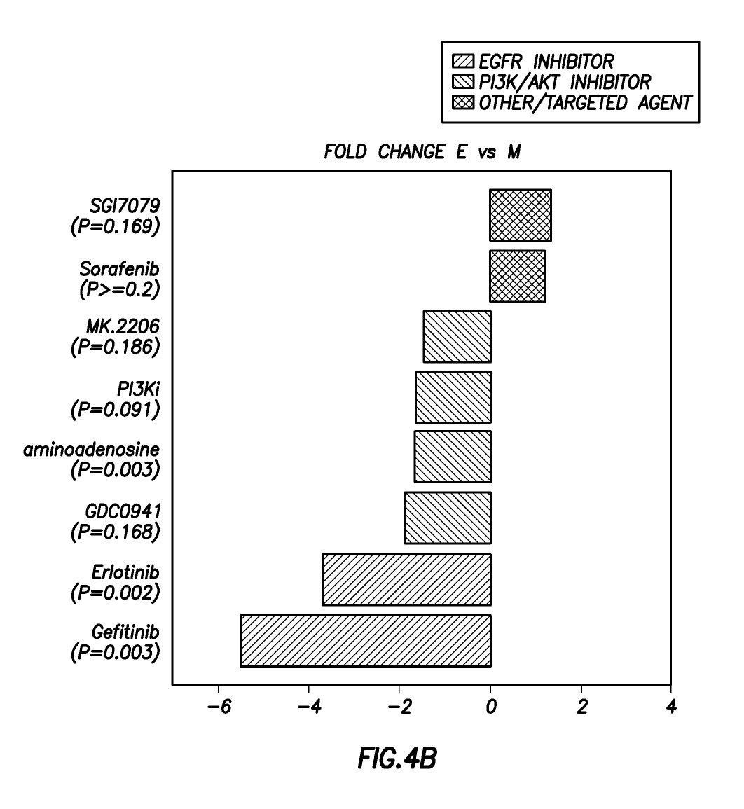

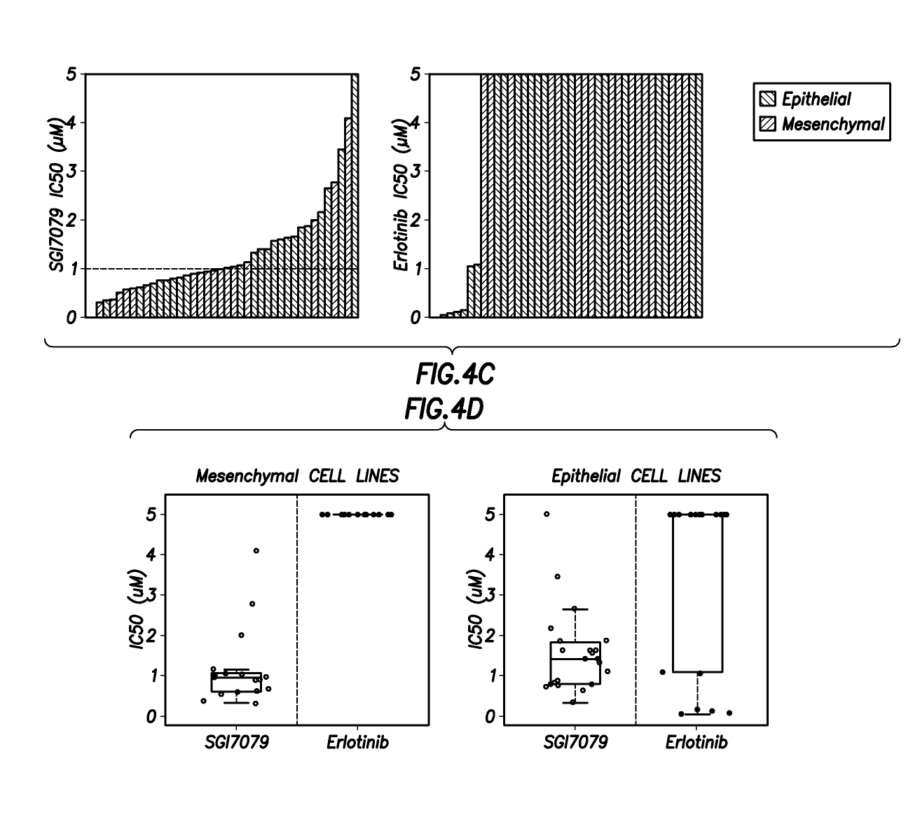

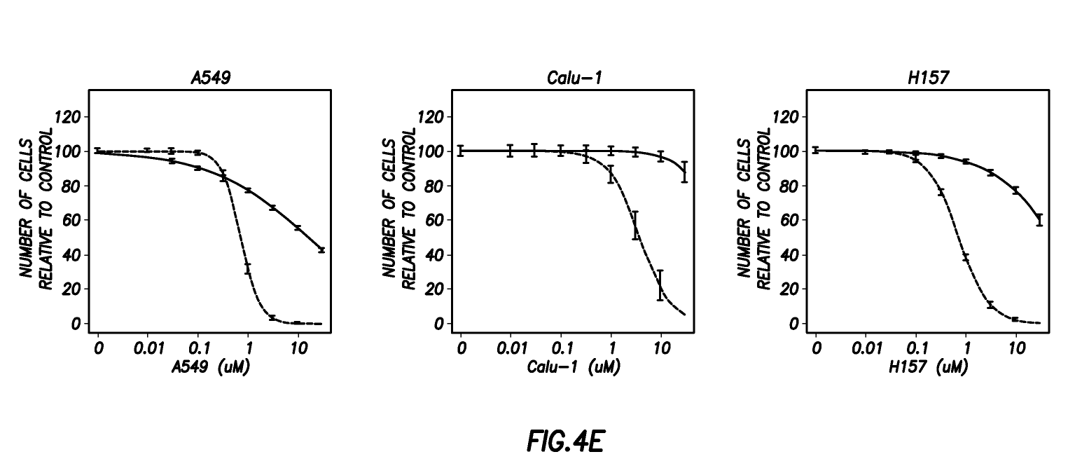

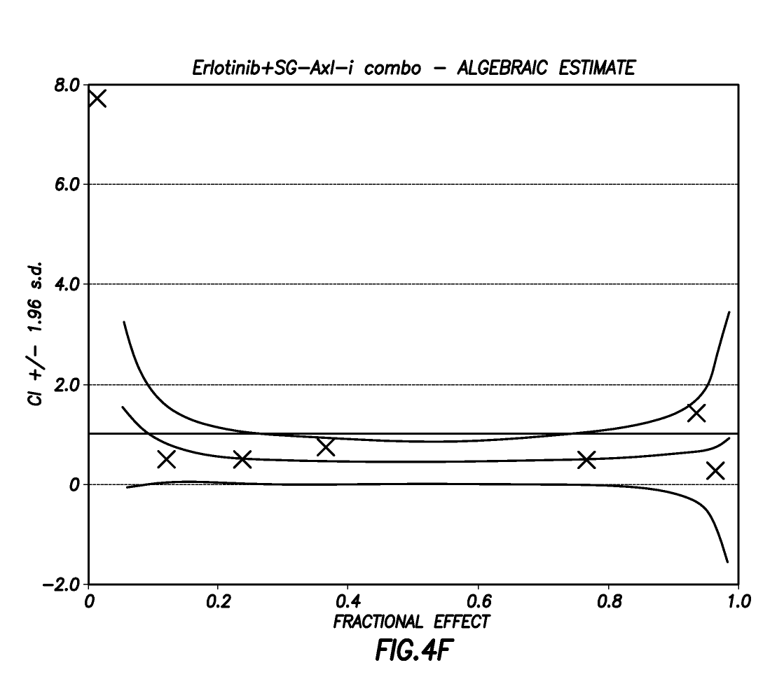

Previously, E-cadherin expression has been associated with greater benefit from erlotinib in NSCLC patients. Yauch R. L., et al., Epithelial Versus Mesenchymal Phenotype Determines In Vitro Sensitivity and Predicts Clinical Activity of Erlotinib in Lung Cancer Patients, Clin Cancer Res. 11:8686-98 (2005); Thomson S., et al., Epithelial to Mesenchymal Transition is a Determinant of Sensitivity of Non-Small-Cell Lung Carcinoma Cell Lines and Xenografts to Epidermal Growth Factor Receptor Inhibition, Cancer Res. 65:9455-62 (2005); Frederick B. A., et al., Epithelial to Mesenchymal Transition Predicts Gefitinib Resistance in Cell Lines of Head and Neck Squamous Cell Carcinoma and Non-Small Cell Lung Carcinoma, Mol Cancer Ther. 6:1683-91 (2007); Nikolova D. A., et al., Cetuxirnab Attenuates Metastasis and U-PAR Expression in Non-Small Cell Lung Cancer: U-PAR and E-Cadherin are Novel Biomarkers of Cetuximab Sensitivity, Cancer Res. 69:2461-70 (2009). Therefore, we tested the association between our EMT signature and cell line sensitivity to erlotinib. Mesenchymal cells were highly resistant to erlotinib, with IC₅₀s 3.7-fold higher in mesenchymal versus epithelial cell lines (p=0.002 by t-test). (FIGS. 4 & 5). Mesenchymal lines were also more resistant to gefinitib (p=0.0003 by t-test, 5.5-fold higher mean IC₅₀ values) (FIGS. 4 & 5).

FIGS. 4A, 4B, 4C, 4D and 4E shows that mesenchymal lines are resistant to EGFR inhibition and P13K pathway inhibition but sensitive to Axl inhibition by SGI-7079. FIG. 4A depicts the relative IC₅₀ levels of targeted agents are shown with p-values corresponding to Wilcoxon rank sum test. FIG. 4 B is the fold difference between mean IC50s in epithelial (E) versus mesenchymal (M) cell lines. FIGS. 4C and 4D show mesenchymal cell lines are relatively more sensitive to SGI-7079 whereas epithelial cell lines are more sensitive to erlotinib. Gray bar (C) denotes 1 uM concentration. FIG. 4E is a representative plot showing increased sensitivity of A549 to combined erlotinib+SGI-7079 versus either drug alone.

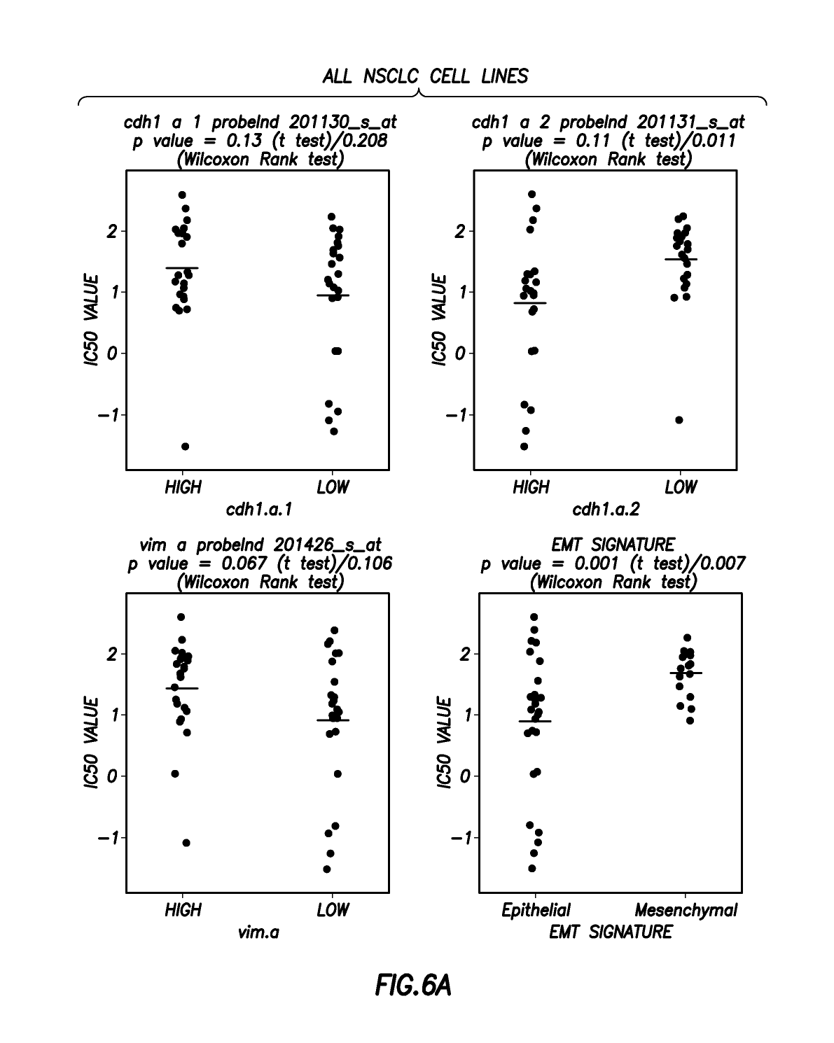

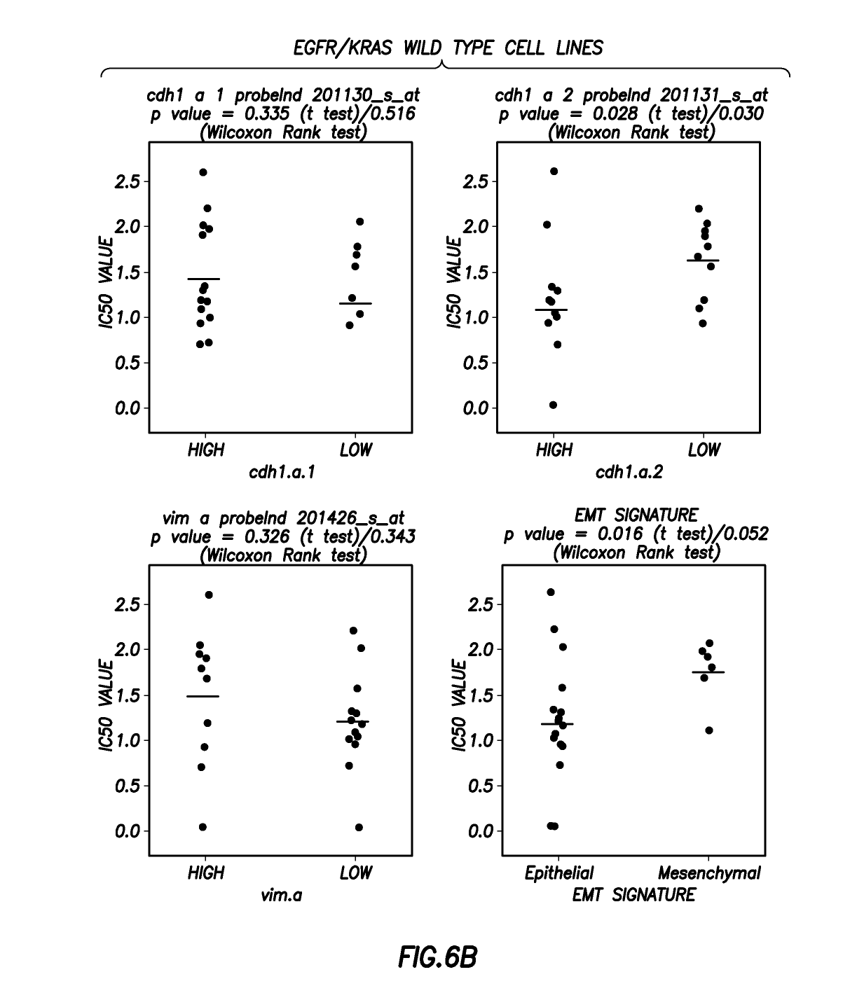

Although cell lines with EGFR activating mutations were among the most sensitive to erlotinib, in the subset with wild-type EGFR and wild-type KRAS, the correlation between EMT signature and erlotinib response was maintained, with significantly greater resistance in mesenchymal lines (p=0.023, 2-fold higher mean IC₅₀ values). Importantly, the EMT signature was a better predictor of erlotinib response than were mRNA probe sets for individual genes such as CDH1 or VIM (FIG. 6).

As with EGFR inhibitors, mesenchymal NSCLC cell lines were also more resistant to PI3K/Akt pathway targeting drugs, such as the selective pan PI3K inhibitor GDC0941 (p=0.068, 1.9-fold higher IC₅₀) and 8-amino-adenosine, an adenosine analog that inhibits Akt/mTOR signaling (p=0.003, 1.7-fold higher IC₅₀) (FIG. 4A, B). Dennison J. B., et al., 8-Aminoadenosine Inhibits Akt/Mtor and Erk Signaling in Mantle Cell Lymphoma, Blood 116:5622-30 (2010); Ghias K., et al., 8-Amino-Adenosine Induces Loss of Phosphorylation of P38 Mitogen-Activated Protein Kinase, Extracellular Signal-Regulated Kinase 1/2, and Akt Kinase: Role in Induction of Apoptosis in Multiple Myeloma, Mol Cancer Ther. 4:569-77 (2005). A trend towards greater resistance was also seen in mesenchymal cells treated with the selective Akt inhibitor MK2206 (p=0.18, 1.5-fold difference IC₅₀), although this did not reach statistical significance. In contrast to EGFR and PI3K inhibitors, mesenchymal cells were not more resistant to other targeted agents, such as sorafenib (p=0.33), suggesting that EMT is not a marker of pan-resistance, but may identify subgroups of cancers more or less likely to respond to inhibition by drugs with distinct pathway targeting or mechanisms of action.

### Axl as a Mesenchymal Target to Reverse EGFR Inhibitor Resistance.

Because the receptor tyrosine kinase Axl was expressed at higher mRNA and protein levels in mesenchymal cell lines (FIG. 3C), we tested the activity of the Axl inhibitor SGI-7079 in mesenchymal versus epithelial NSCLC lines. In keeping with their higher target expression, mesenchymal cells were 1.3-fold more sensitive overall to Axl inhibition, although this did not reach statistical significance (p-value 0.17 by t-test) (FIGS. 4A & 4B, & FIG. 7).

FIG. 7 shows the improved 8-week disease control in BATTLE patients with epithelial signatures treated erlotinib. FIG. 7A shows that BATTLE (all treatment arms) were classified as mesenchymal or epithelial-like based on the EMT signature. FIG. 7B shows that among patients with wild type EGFR and KRAS treated with erlotinib, 8-week disease control appeared superior in patients with epithelial tumor signatures (p=0.052) (defined as the first principal component of the EMT signature below the median). As shown in FIG. 7C, there was no significant difference in 8 week disease control between epithelial and mesenchymal tumors in other treatment arms.

We then compared the sensitivity of mesenchymal cells to SGI-7079 versus erlotinib (FIGS. 4C & 4D). Mesenchymal cells were uniformly resistant to erlotinib, but relatively sensitive to SGI-7079 (p<0.001 by Wilcoxon test). Next, we tested whether Axl inhibition could reverse mesenchymal cell resistance to EGFR inhibition, since Axl inhibition has been shown to reverse the mesenchymal phenotype in other epithelial cancers. Koorstra J. B., et al,. The Axl Receptor Tyrosine Kinase Confers an Adverse Prognostic Influence in Pancreatic Cancer and Represents a New Therapeutic Target, Cancer Biol Ther. 8:618-26 (2009). Cells expressing high levels of Axl were sensitive to SG1-7079 (range 0.74-4.29 μM, mean 1.3 μM), but not to single agent erlotinib (range 13.5->100 μM, mean 77 μM). However, when combined, the addition of Axl inhibition (SGI-7079) to EGFR inhibition (erlotinib) (3:1 ratio of erlotinib to SGI-7079) resulted in a striking synergistic effect as demonstrated by the Chou-Talalay combination index (Cl 0.46-0.72) in four of six cell lines.

TABLE 2
Axl Inhibition Reverses EGFR Resistance in Mesenchymal Cell Lines.

| | A549 | Calu-1 | H157 | H1299 | H460 | H2882 |
|---|---|---|---|---|---|---|
| Erlotinib IC₅₀ | 13.54 | >100 | 48.50 | >100 | >100 | >100 |
| (μM) | | | | | | |
| SGI-7079 IC₅₀ | 0.92 | 2.44 | 0.74 | 1.74 | 2.01 | 4.29 |
| (μM) | | | | | | |
| Combination: | 1.07 + 0.35 | 13.86 + 4.47 | 1.46 + 0.47 | 3.76 + 1.21 | 3.53 + 1.14 | 16.44 + 5.30 |
| Erlotinib + SGI- | | | | | | |
| 7079 IC₅₀ (μM) | | | | | | |
| CI @IC₅₀ | 0.46 | >1.00* | 0.67 | 0.72 | 0.57 | >1.00* |

*for Calu-1 and H2882, the combination was synergistic at higher concentration of SGI-7079., CI, combination index.
Johnson F. M., et al., Abrogation of Signal Transducer and Activator of Transcription 3 Reactivation After Src Kinase Inhibition Results in Synergistic Antitumor Effects, Clin Cancer Res. 13: 4233-44 (2007).

In two cell lines with highest Axl protein expression (Calu-1 and H2882), the combination was synergistic at higher concentrations of SGI-7079, possibly reflecting a need for higher dosing in cells with higher target expression levels.

EMT Signature in Patients with Relapsed or Metastatic NSCLC.

Finally, we tested the EMT signature in 139 previously-treated NSCLC patients with advanced NSCLC enrolled in the BATTLE-1 trial (Biomarker-integrated Approaches of Targeted Therapy for Lung Cancer Elimination). Kim E. S. H. R., The BATTLE Trial: Personalizing Therapy for Lung Cancer, Cancer Discovery 1:43-51 (2011). Consistent with the cell line data—and despite all patients having advanced, metastatic disease—a majority of patients (approximately 2/3) had epithelial signatures (FIG. 7). However, EGFR and KRAS mutations were distributed more evenly between the two patient groups, possibly because of prior therapy (e.g., previous EGFR inhibitors in EGFR mutant patients). Among 101/139 clinically evaluable patients (all treatment arms), the EMT signature was not prognostic of 8-week disease control or improved progression-free survival (PFS) (p>0.4 by t-test). Al-Hamidi H., et al., To Enhance Dissolution Rate of Poorly Water-Soluble Drugs: Glucosamine Hydrochloride as a Potential Carrier in Solid Dispersion Formulations, Colloids Surf B Biointerfaces 76:170-78 (2010). However, in erlotinib-treated patients, those with wildtype EGFR and KRAS who had epithelial signatures were more likely to have 8-week DC (p=0.05, by t-test)(FIG. 7B). Specifically, six out of seven BATTLE patients with DC at 8 weeks had an epithelial EMT signature, whereas only 1/5 patients with mesenchymal signatures had DC. In contrast, the signature was not associated with differences in DC in other treatment arms (e.g., sorafenib), suggesting the EMT signature may be a marker of erlotinib activity in EGFR wild-type/KRAS wild-type tumors, and not simply a prognostic marker of a less aggressive tumor phenotype

### Discussion

EMT is a pervasive process among epithelial cancers that has been linked to morphologic changes, increased invasiveness, and metastatic potential. While a number of EMT markers have been identified, no robust gene signature capable of use across multiple platforms has been established. Furthermore, the mesenchymal phenotype has been linked with resistance to EGFR inhibitors, but it is unknown how EMT affects response to other drugs and effective therapeutic strategies for targeting mesenchymal cells are needed.

To address these needs, we developed and validated a robust, platform-independent gene expression signature capable of classifying NSCLC as epithelial or mesenchymal. The signature was selected using probes with high cross-platform correlations to increase the likelihood that the signature could be applied to different types of mRNA arrays or emerging technologies. The success of this approach was demonstrated in independent testing sets, with essentially identical classification of cell lines profiled on Affymetrix, Illumina v2 and v3 arrays. An integrated analysis of mRNA and proteomic expression confirmed strong correlation of the EMT signature with E-cadherin protein levels. Additionally, higher expression of activated EGFR signaling proteins was observed in epithelial cell lines. Moreover, as predicted, EGFR mutant cells all demonstrated an epithelial signature.

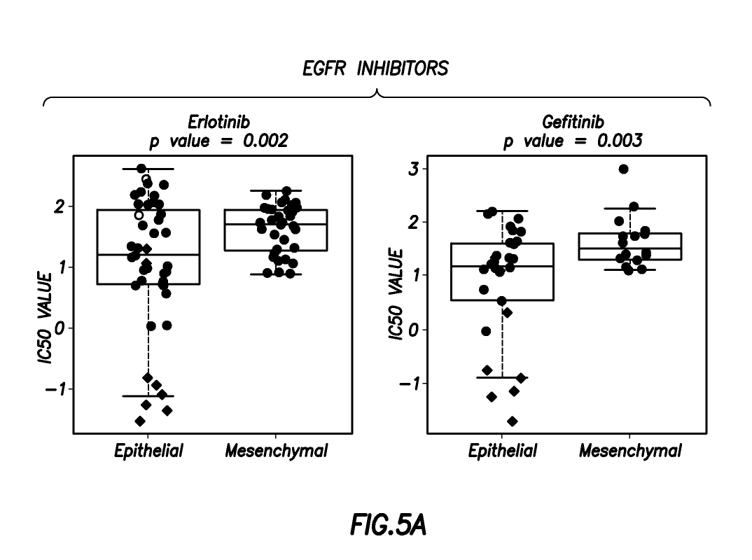

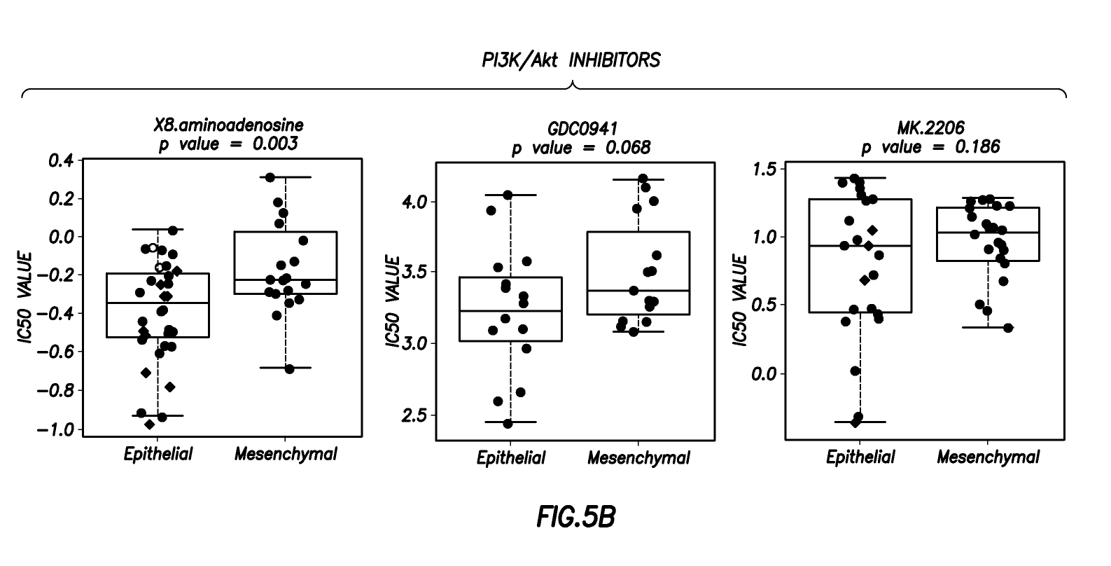

To investigate whether other drugs may preferentially target epithelial or mesenchymal cells we assessed the activity of several targeted drugs used commonly in NSCLC or in current clinical development. Consistent with prior studies, epithelial cells demonstrated greater sensitivity to the EGFR inhibitors erlotinib and gefitinib in vitro, independent of EGFR mutation status, while mesenchymal cells were highly resistant (FIG. 4 and FIG. 5A). Notably, the ability of the EMT signature to predict response to EGFR inhibitors was independent of EGFR mutations. Here for the first time we also showed a similar “epithelial-bias” in drugs targeting the PI3K/Akt pathway such that these drugs had significantly greater activity in epithelial as compared to mesenchymal lines (FIG. 5B). These results suggest that a mesenchymal signature may be a good predictor of resistance to both EGFR and P13K/Akt pathway inhibitors, akin to KRAS mutations for EGFR TKIs. In contrast, there was no association between EMT status and drug response for sorafenib in cell lines or patients treated on the BATTLE trial (FIG. 4 and FIG. 5).

Next, we investigated Axl as a potential therapeutic target for the mesenchymal phenotype. We observed higher levels the receptor tyrosine kinase Axl in the mesenchymal phenotype at both the mRNA and protein level (FIGS. 3B & 3C). Axl has been associated with poor prognosis and invasiveness in pancreatic cells and with metastasis in preclinical NSCLC models. Koorstra J. B., et al., The Axl Receptor Tyrosine Kinase Confers an Adverse Prognostic Influence in Pancreatic Cancer and Represents a New Therapeutic Target, Cancer Biol Ther. 8:618-26 (2009); Ye X., et al., An Anti-Axl Monoclonal Antibody Attenuates Xenograft Tumor Growth and Enhances the Effect of Multiple Anticancer Therapies, Oncogene 29:5254-64 (2010). It has also been linked to EMT and Her-2 inhibitor resistance in breast cancer but has not been identified as an EMT marker in NSCLC. Liu L., et al., Novel Mechanism of Lapatinib Resistance in HER2-Positive Breast Tumor Cells: Activation of AXL, Cancer Res. 69:6871-8 (2009). We therefore investigated the effects of Axl inhibition on mesenchymal cells and EGFR inhibitor resistance and found that, unlike the epithelial-bias demonstrated by EGFR or P13K inhibitors, the Axl inhibitor demonstrated a trend towards a mesenchymal-bias (FIGS. 4A-D). Moreover, inhibition of Axl sensitized otherwise-resistant mesenchymal NSCLC lines to the EGFR inhibitor erlotinib. (FIG. 4E). Therefore, in addition to single agent activity, Axl inhibition has a role in reversing EMT-associated EGFR inhibitor resistance, supporting further investigation of combined Axl and EGFR inhibition.

Finally, we tested the EMT signature in refractory NSCLC patients treated with erlotinib or sorafenib in the BATTLE study. Among erlotinib-treated patients (wild-type EGFR and KRAS), those with 8-week disease control, the primary study endpoint, had a more epithelial phenotype than those who did not have DC control (p=0.05, by t-test) (FIG. 7B). Al-Hamidi H., et al., To Enhance Dissolution Rate of Poorly Water-Soluble Drugs: Glucosamine Hydrochloride as a Potential Carrier in Solid Dispersion Formulations, Colloids Surf B Biointerfaces 76:170-78 (2010). Consistent with the preclinical studies, there was no difference in EMT score among sorafenib-treated patients with or without DC, and EMT was not prognostic in the overall population, providing evidence that EMT is not merely a pan-resistance or negative prognostic marker in this context but rather may potentially be informative for drug selection.

This study established a robust, cross-platform EMT signature capable of classifying NSCLC cell lines and patient tumors as epithelial or mesenchymal. Consistent with prior studies, the mesenchymal phenotype is associated with resistance to EGFR inhibitors both in vitro and in patients with wild-type EGFR treated with erlotinib, a subgroup for which there is no established predictive marker. Similarly, we also showed that PI3K/AKT inhibitors are more active in epithelial cells. Finally, we identify Axl as a novel EMT marker in NSCLC and demonstrate that Axl inhibitors are active against cells with a mesenchymal phenotype and can reverse EGFR inhibitor resistance associated in mesenchymal cells. Together these findings suggest that assessment of EMT status may guide drug selection in NSCLC patients and dual Axl/EGFR inhibition may be an effective targeted strategy for overcoming EGFR inhibitor resistance associated with the mesenchymal phenotype. These findings merit further investigation in future clinical trials.

### Example II

### Refinement EMT Signature—76 to 35 Genes

### Materials/Methods

The EMT signature was derived in 54 DNA fingerprinted NSCLC cell lines profiled on Affymetrix U133A, B, and Plus2.0 arrays and tested on the Illumina WGv2 and WGv3 platforms and in an independent set of head and neck cancer lines (HNC). E-cadherin and other protein levels were quantified by reverse phase protein array and correlated with the first principal component of the EMT signature. IC50s were determined for NSCLC cell lines by MTS assay. Response to erlotinib was evaluated in patients treated in the BATTLE clinical trial using eight-week disease free status and progression free survival.

In the original EMT signature, genes were selected based on two criteria. First, they must be correlated with one of four EMT genes (CDH1, VIM, FN1 and CDH2). Second, they must be biomodally distributed. A third requirement was added to improve the signature. The third criteria is that the genes included in the signature come from “good quality” probes-defined as those probes having a correlation between Affymetrix and Illumina platform of r greater than 0.90. This refines the signature to the smallest number of genes with the greatest contribution to the EMT signature.

The classification of each cell line as epithelial or mesenchymal remained the same between the original and the refined signature, suggesting that the refined signature includes the “core EMT genes” contributing most significantly to the EMT signature.

### Results

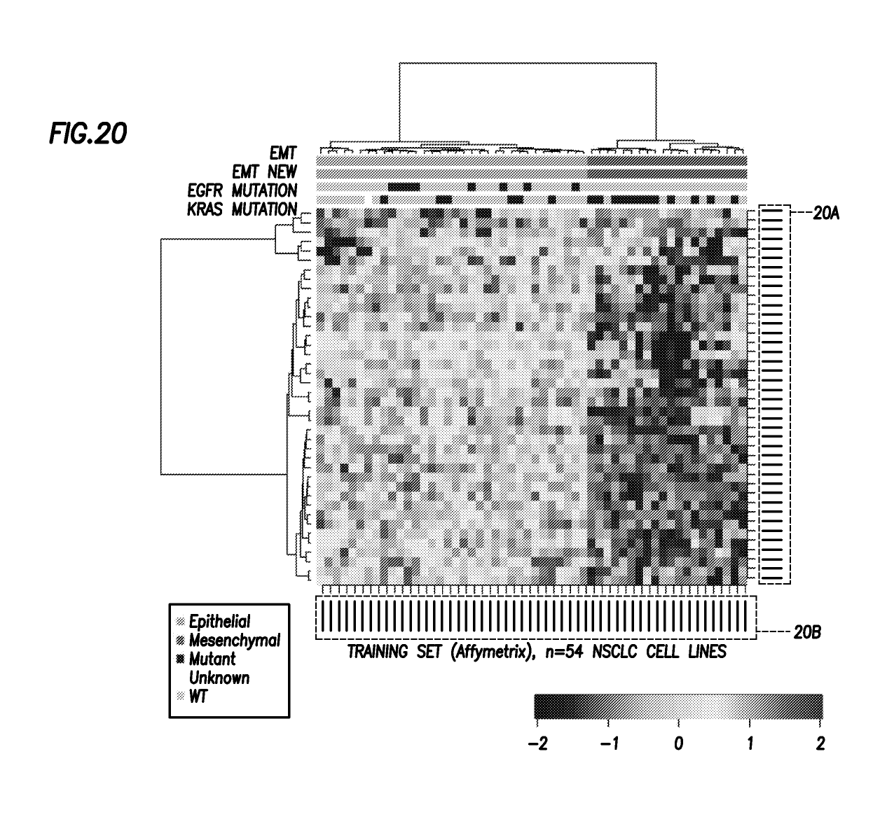

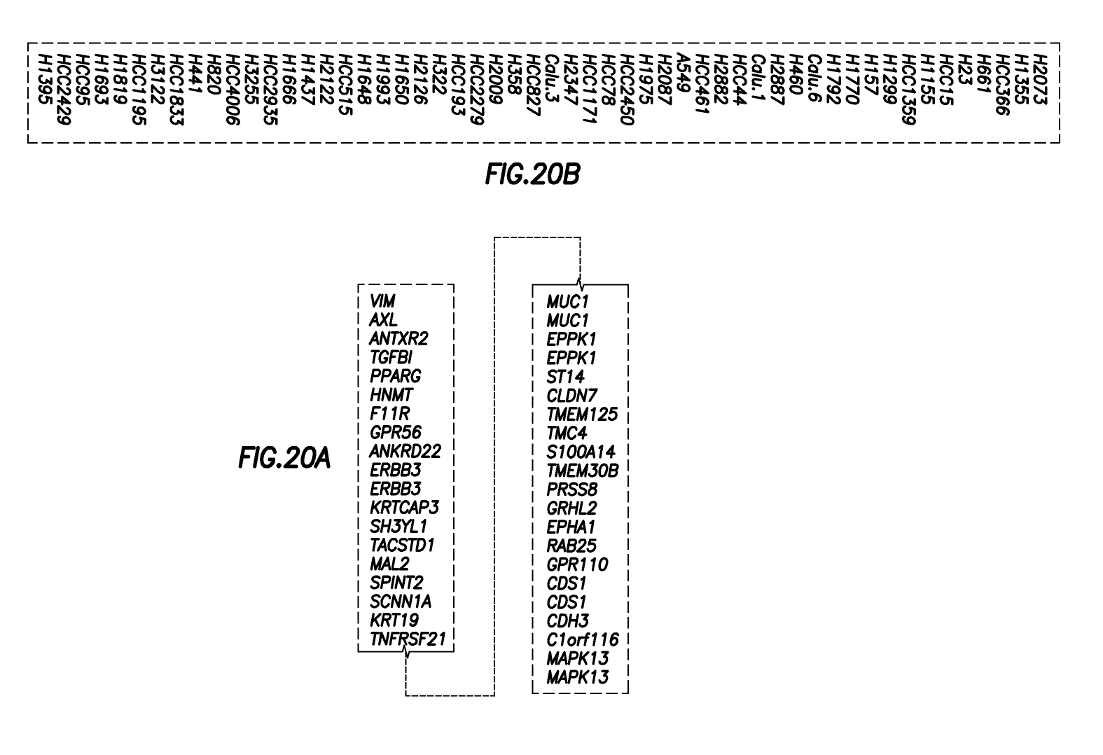

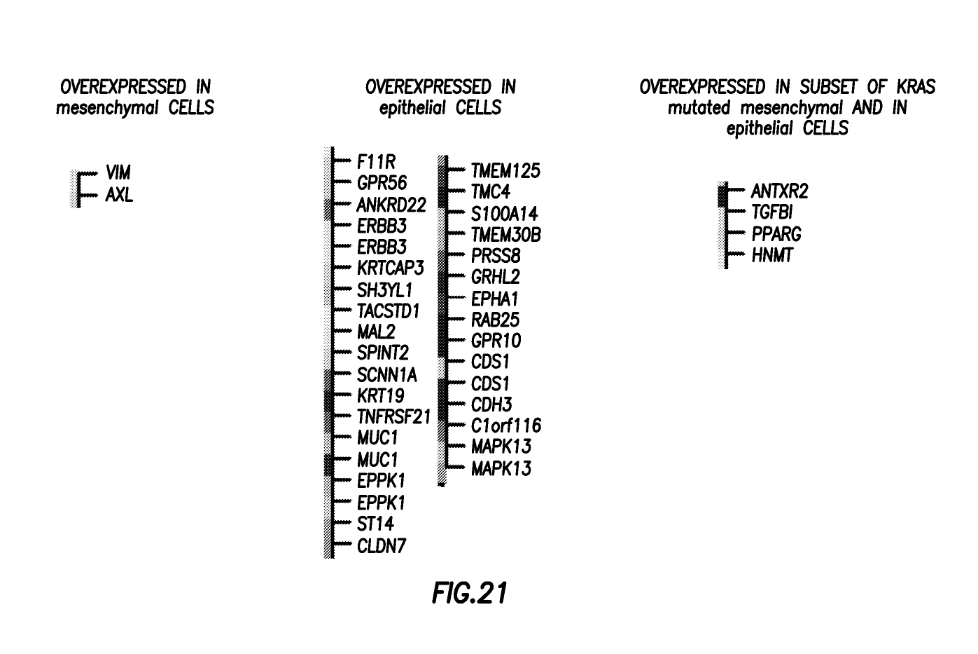

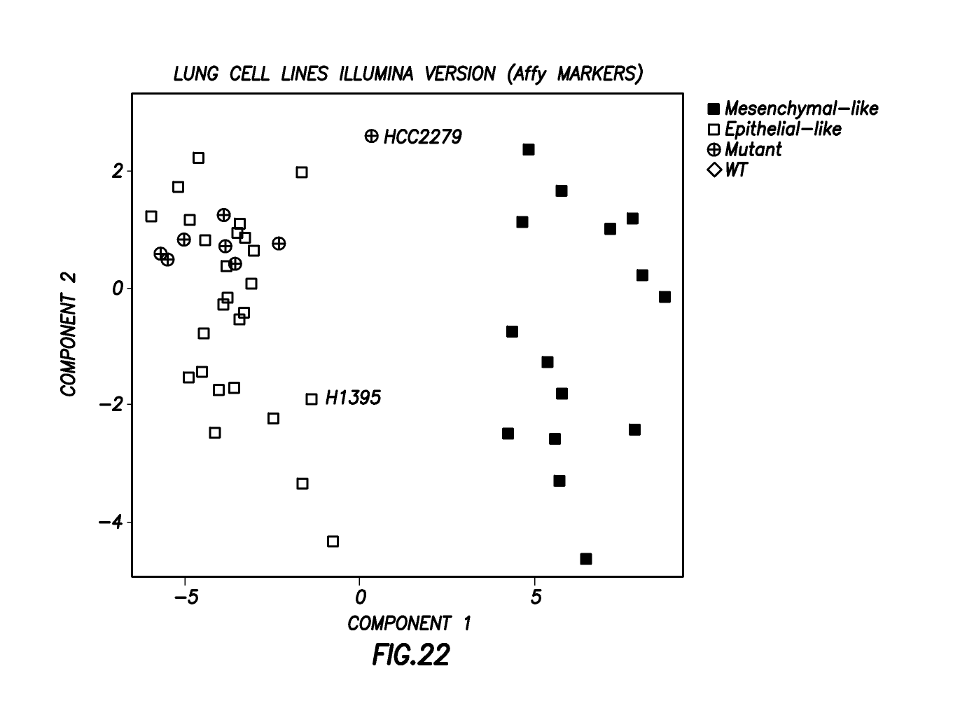

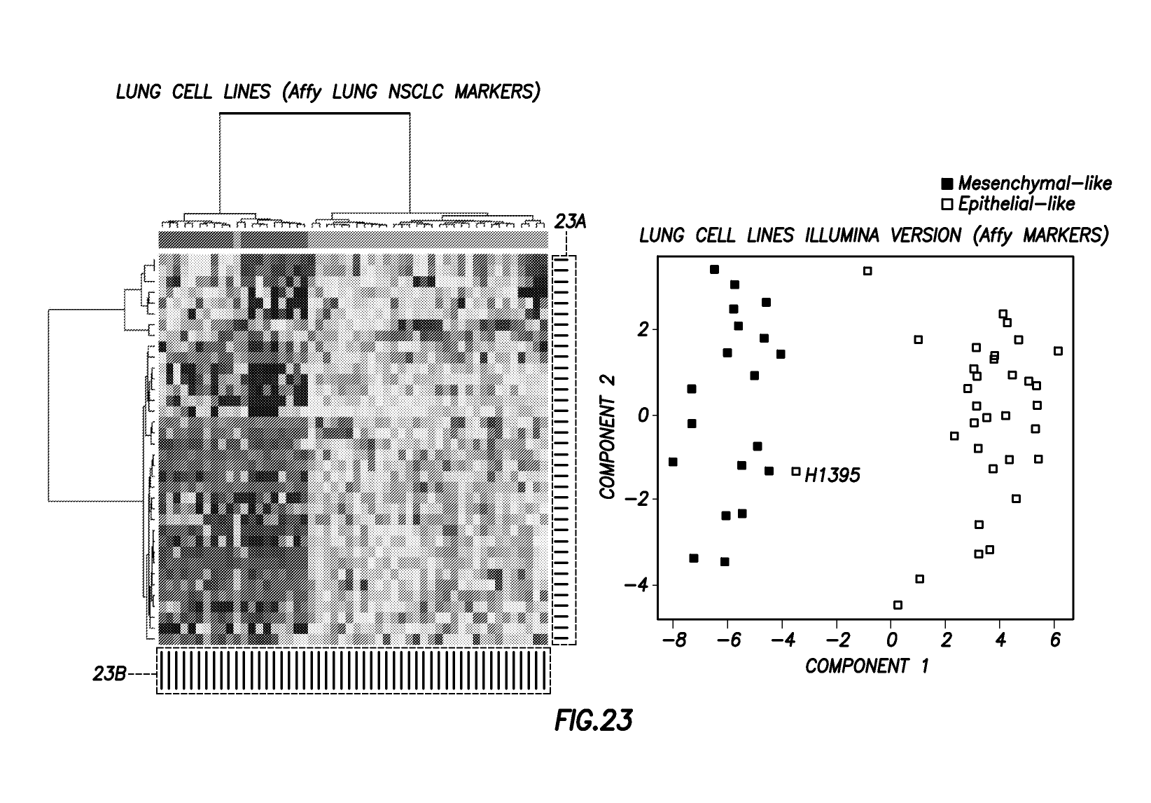

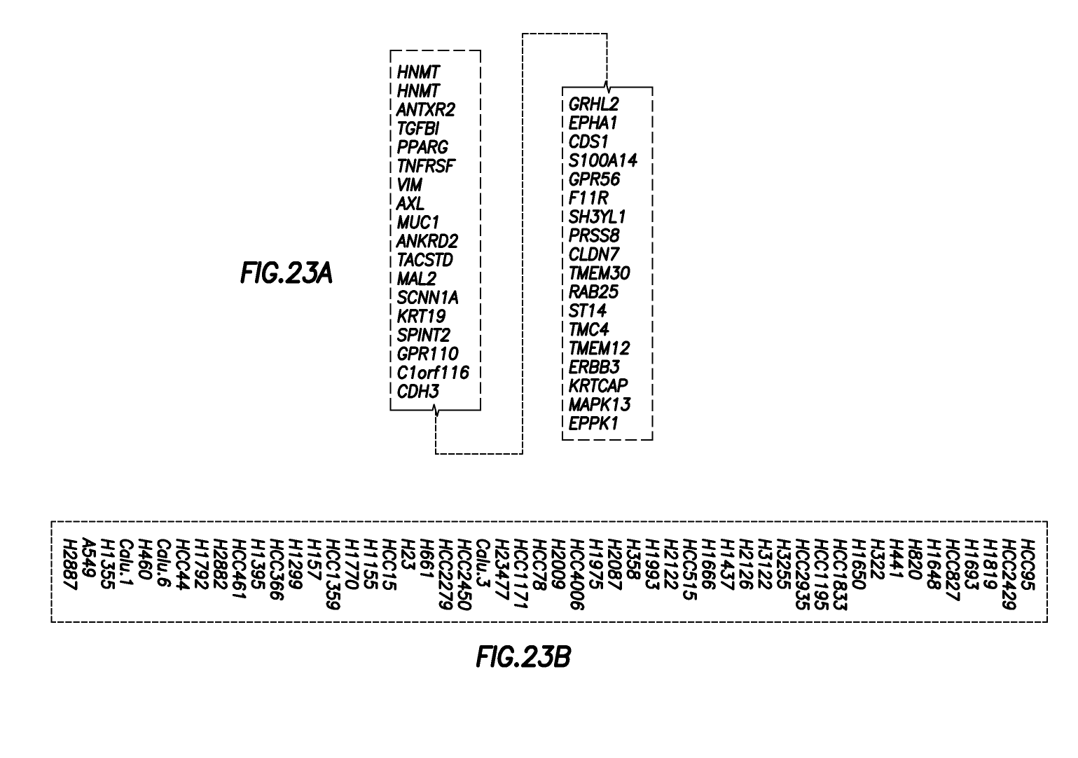

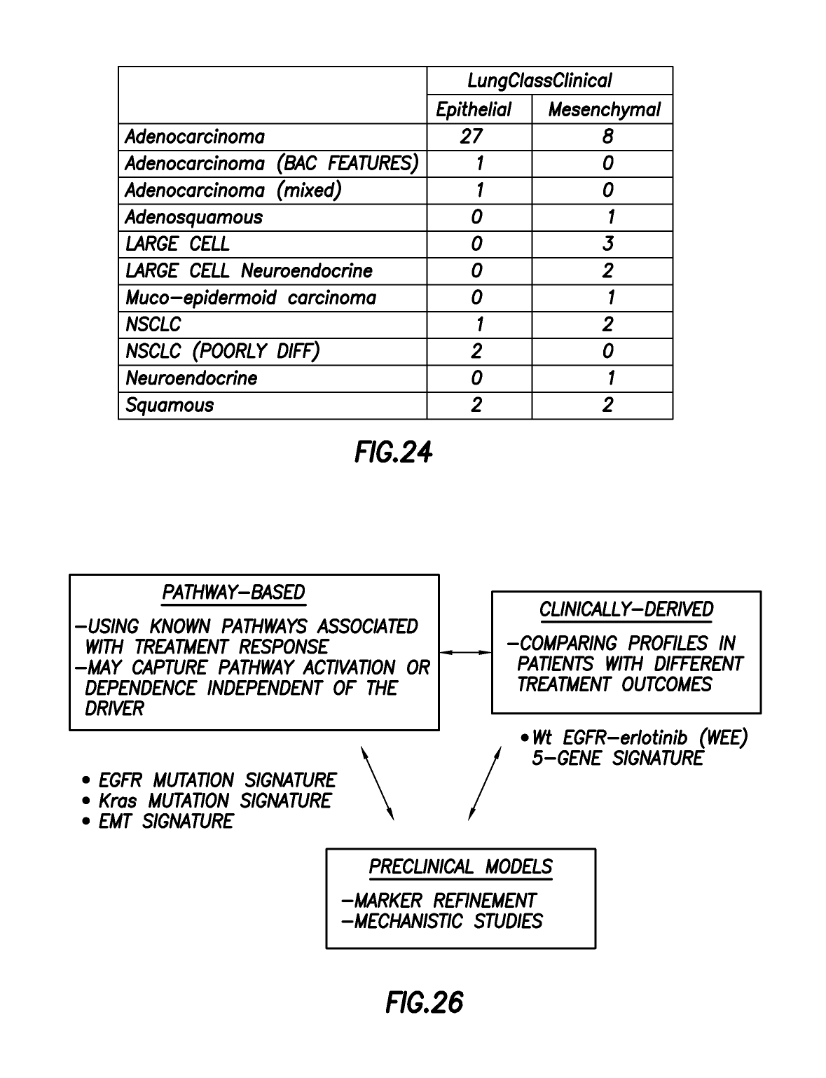

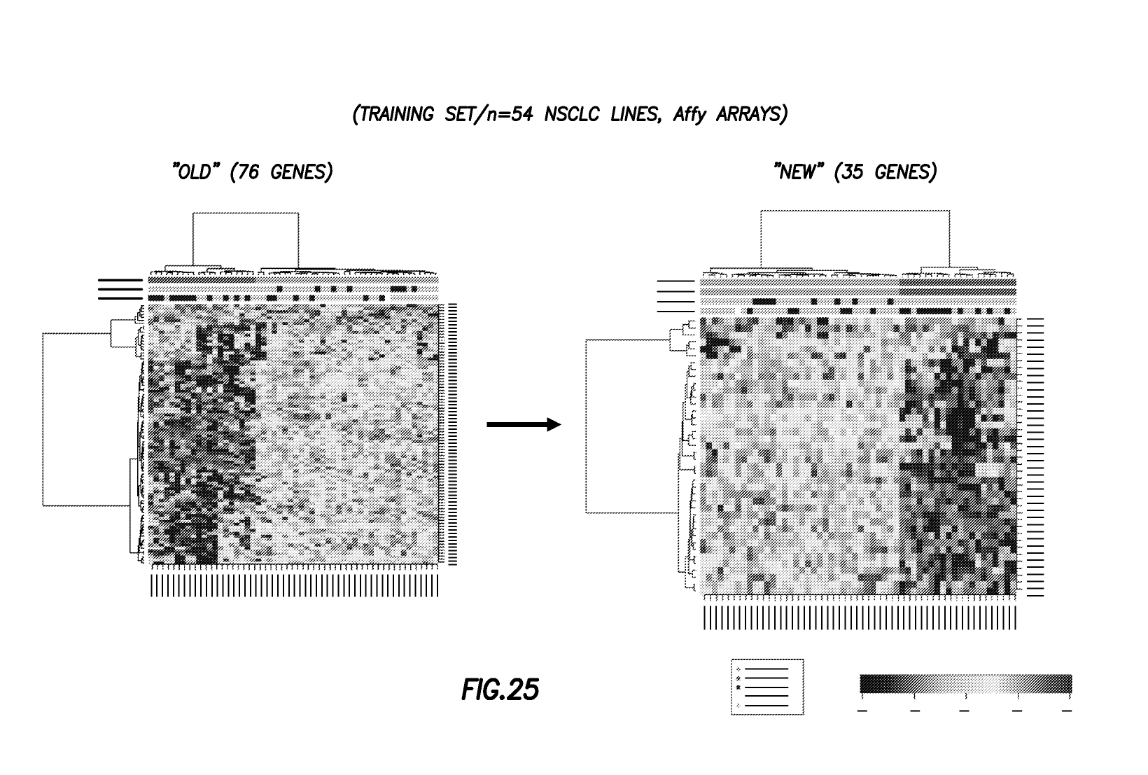

Expression of 35 genes (the EMT signature) correlated with mRNA expression of known EMT markers E-cadherin, vimentin, N-cadherin, or fibronectin 1 and expression was bimodally distributed across the NSCLC panel. FIG. 25. Classification of the NSCLC lines as epithelial or mesnchymal by the EMT signature agreed for 51/52 cell lines tested on both Affymetrix and Illumina platforms. (FIGS. 20, 22 & 23). In an independent validation set of 62 HNC lines, the signature identified a subset of six mesenchymal cell lines. (FIG. 21). The EMT signature score correlated well with E-cadherin protein levels in NSCLC (r=0.90) and HNC (r=0.73).

mRNA levels for Axl, a tyrosine kinase receptor associated with EMT in breast cancer, had the most negative correlation with E-cadherin (r=−0.45) of any signature gene after ZEB 1 and vimentin and was positively correlated with vimentin (r=0.60) and N-cadherin (r-0.54) expression. Higher Axl total protein was confirmed in NSCLC and HNC mesenchymal-like cell lines. Classification as mesenchymal by the EMT signature was more strongly correlated with NSCLC erlotinib resistance (p=0.028) than E-cadherin mRNA or protein level. In contrast, an epithelial classification by the EMT signature was associated with improved 8-week disease control and PFS.

### Example III

### The Five-Gene Signature

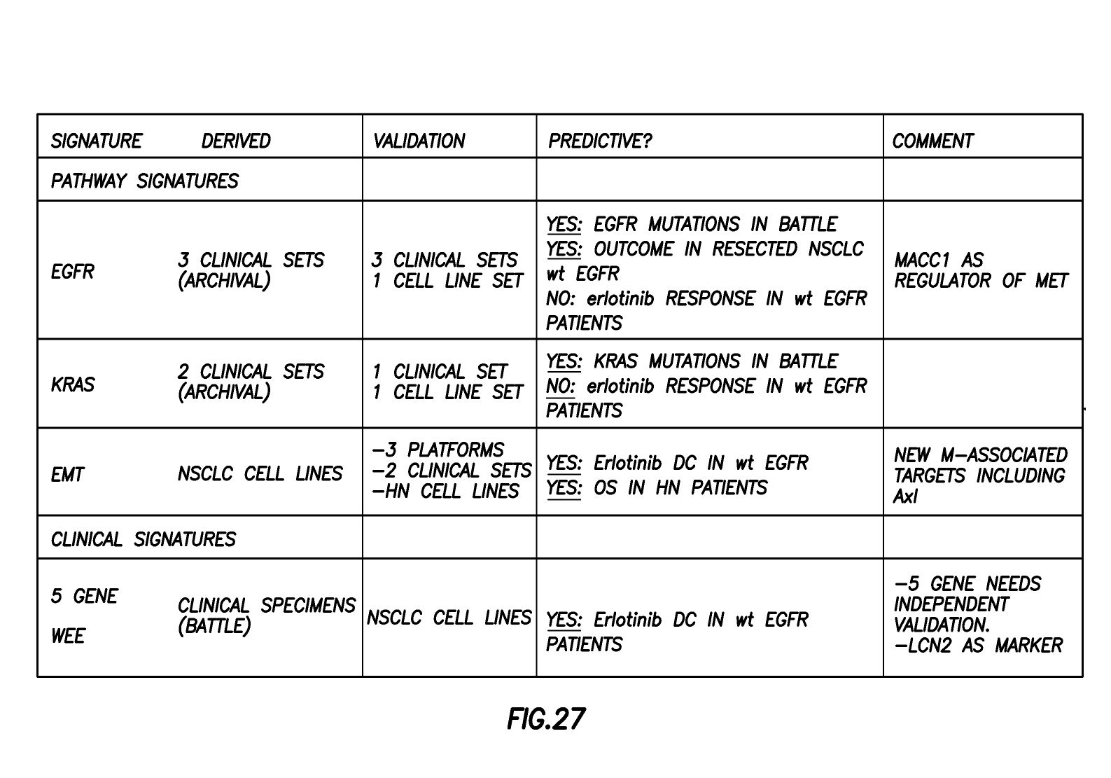

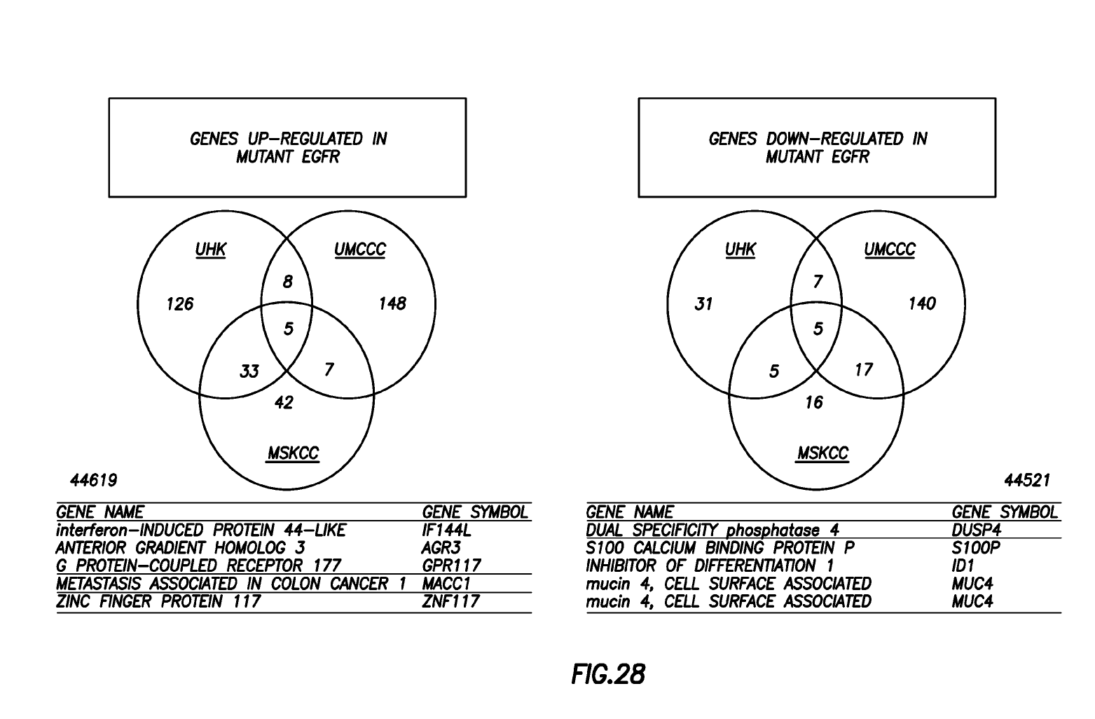

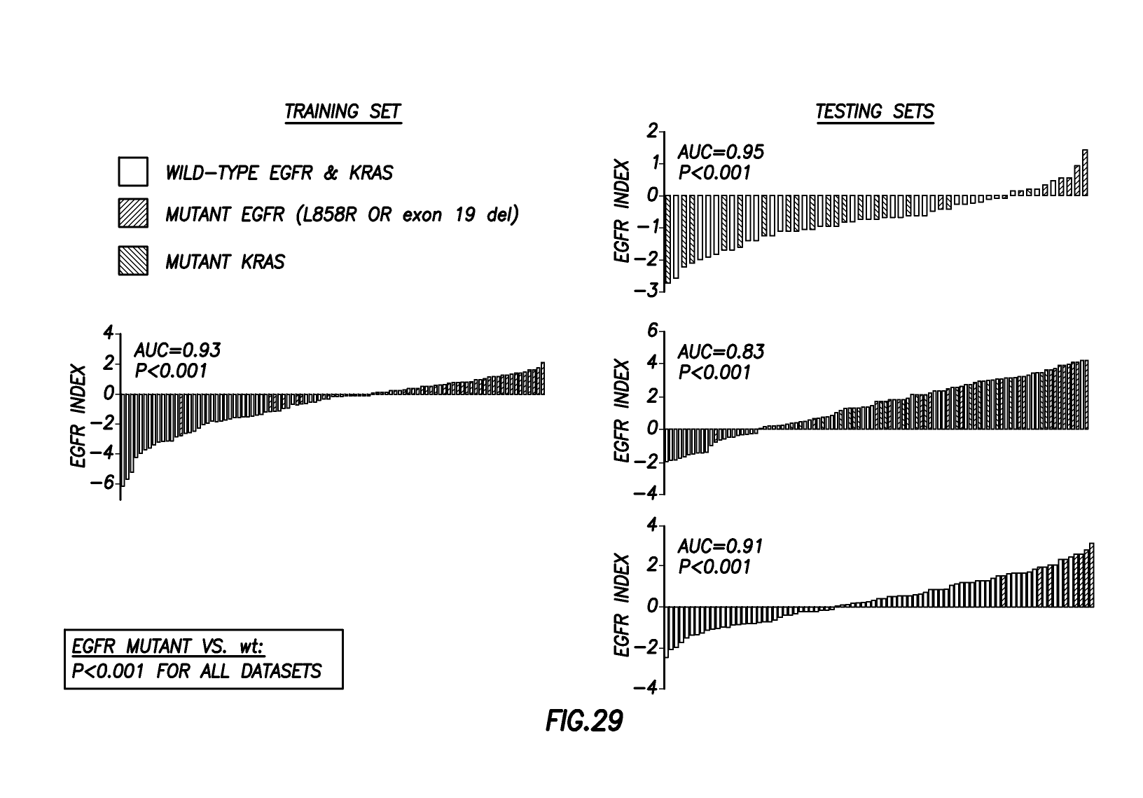

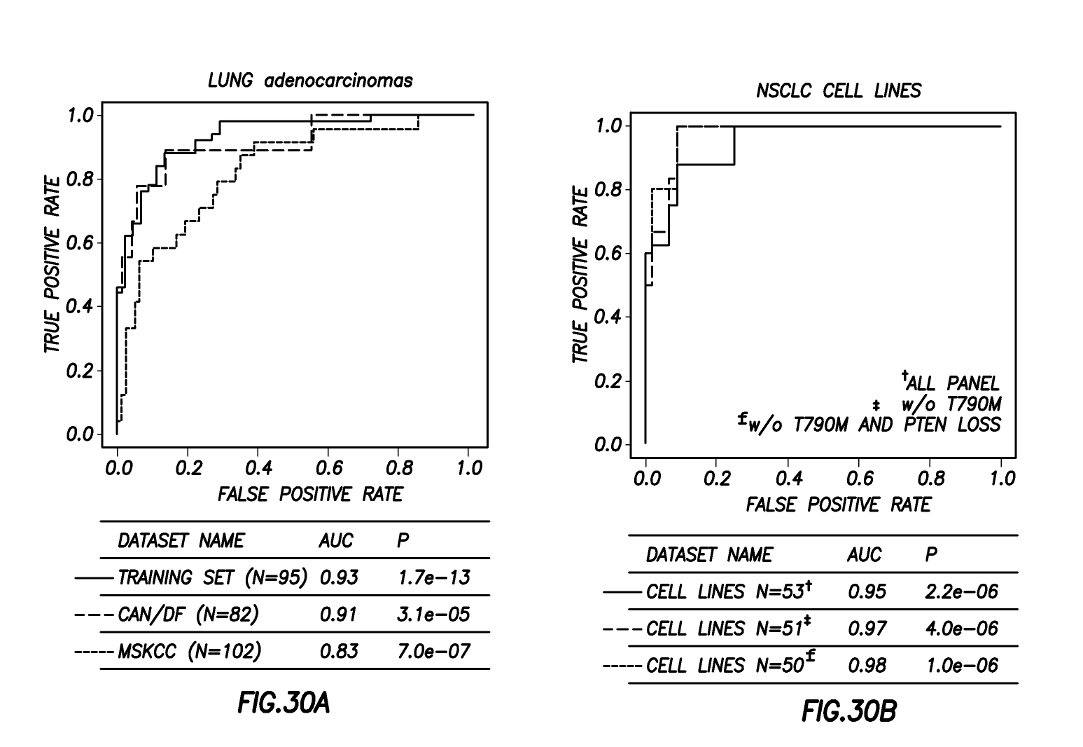

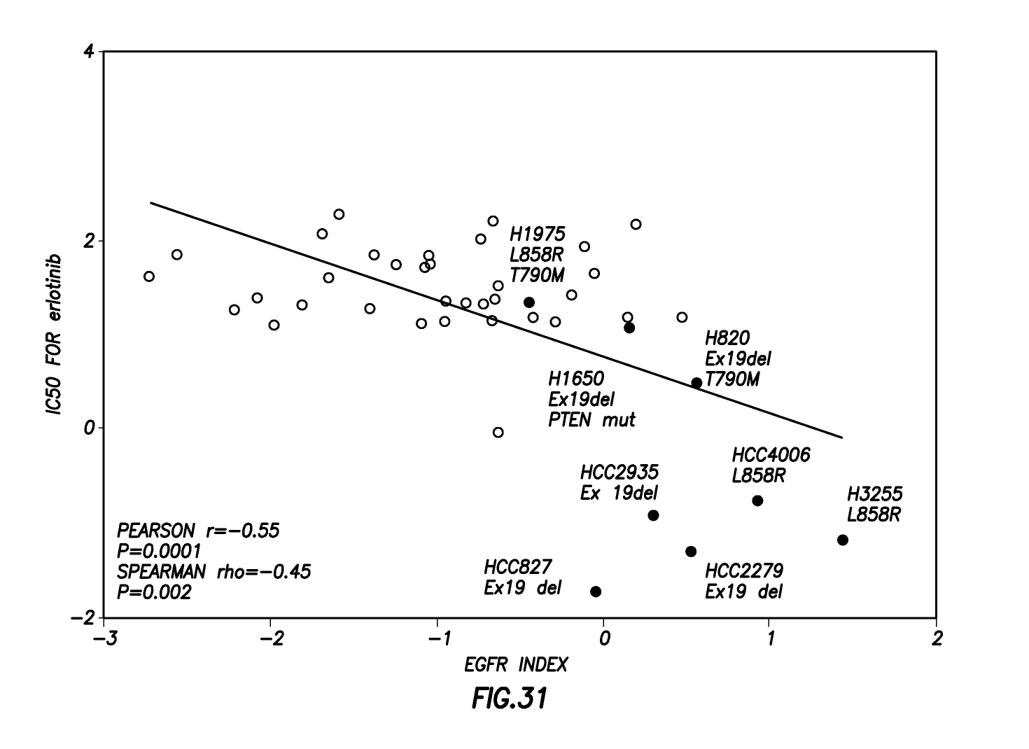

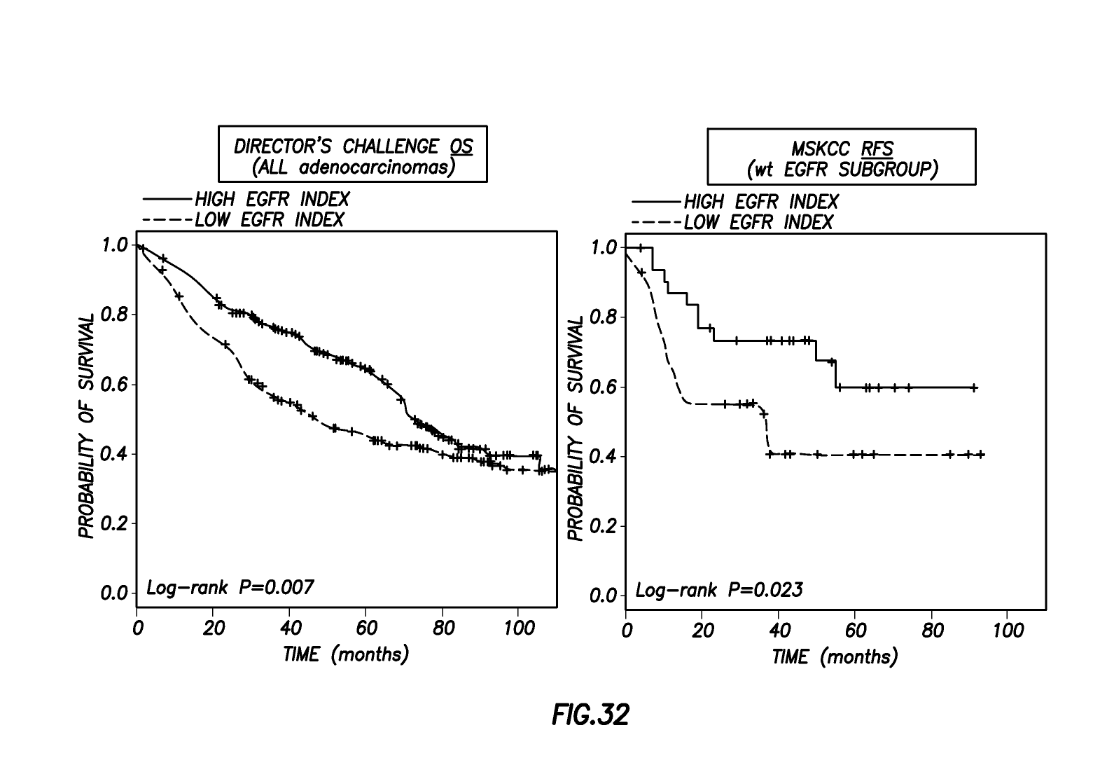

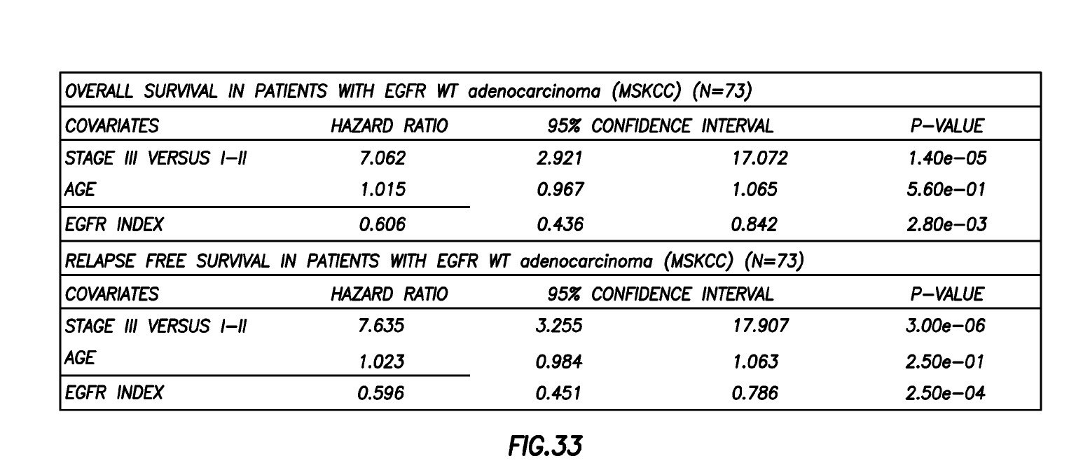

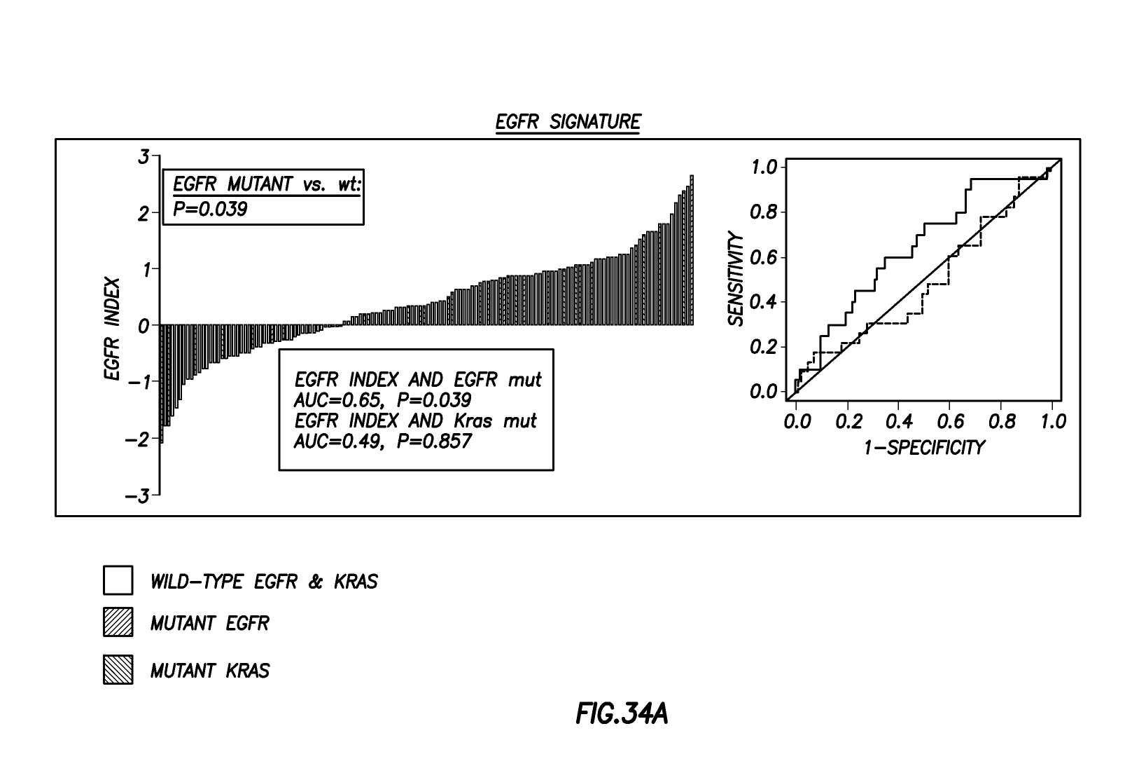

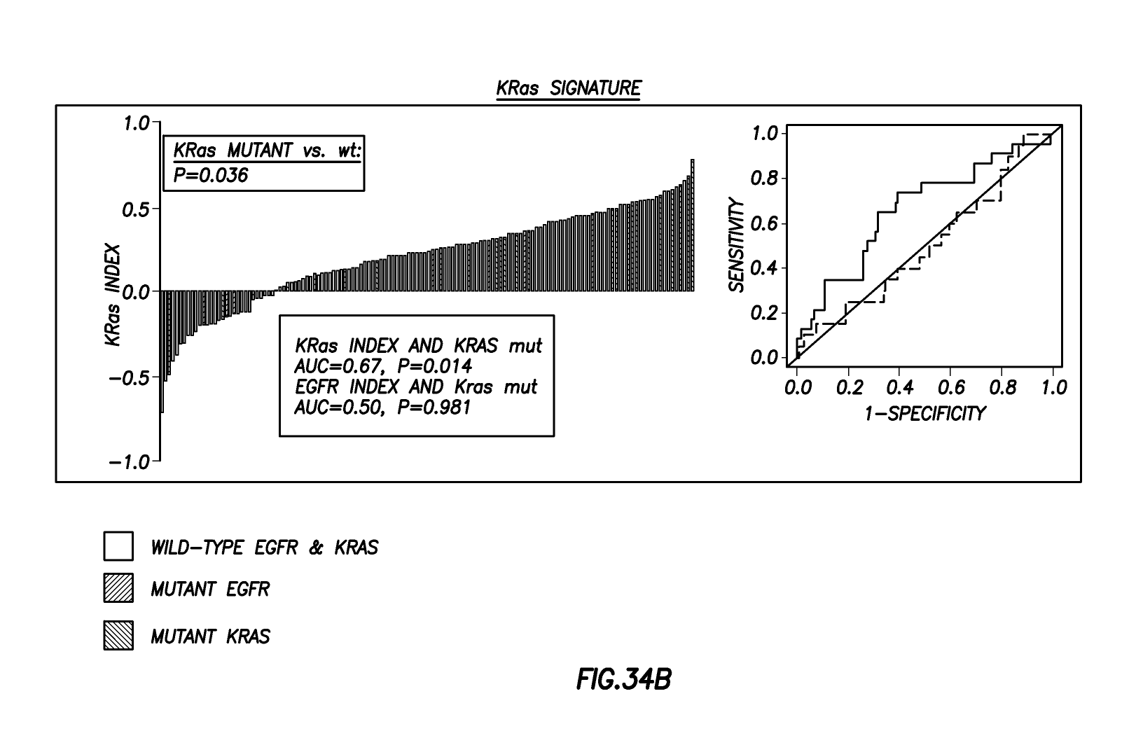

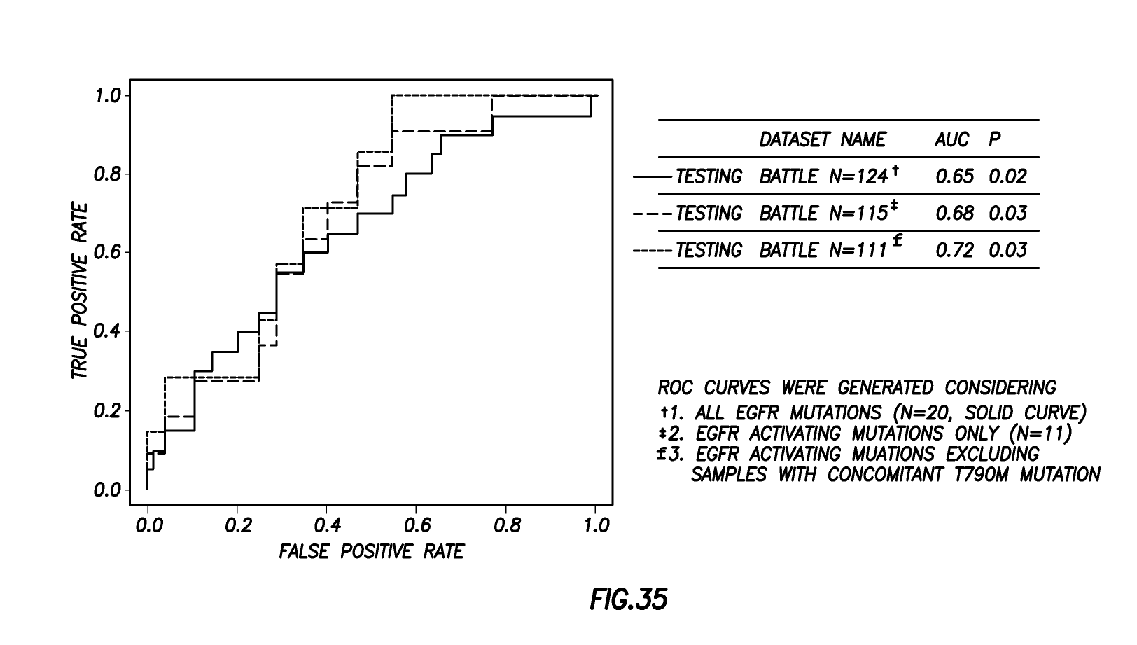

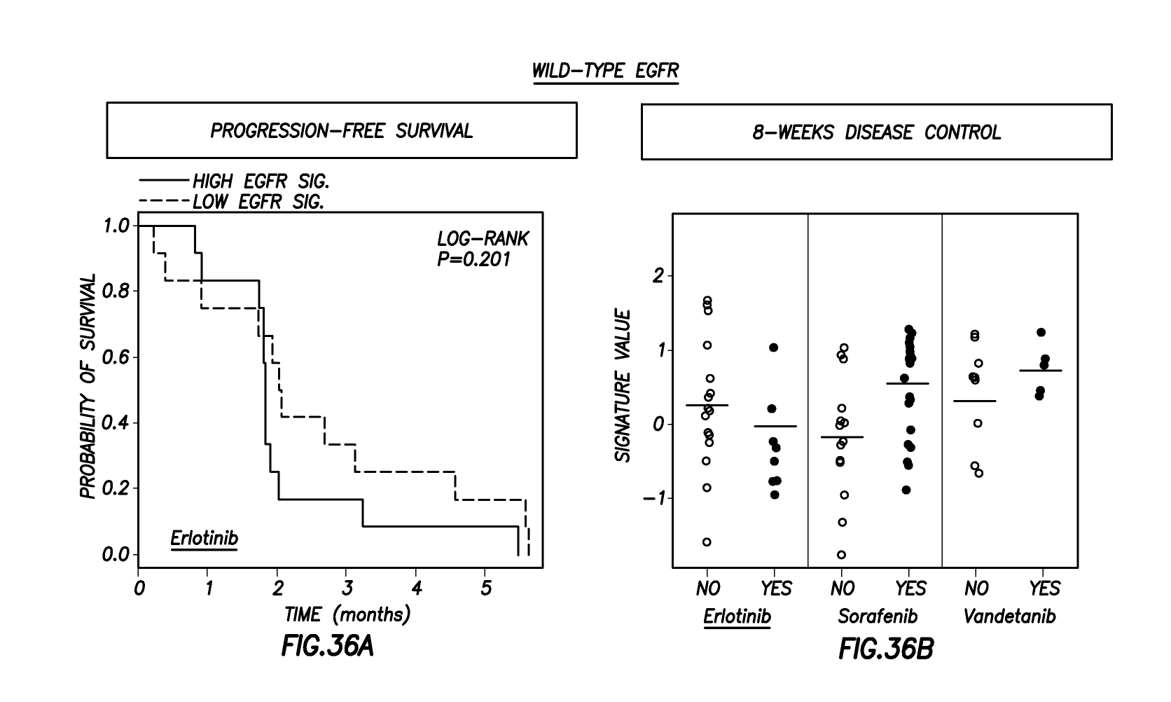

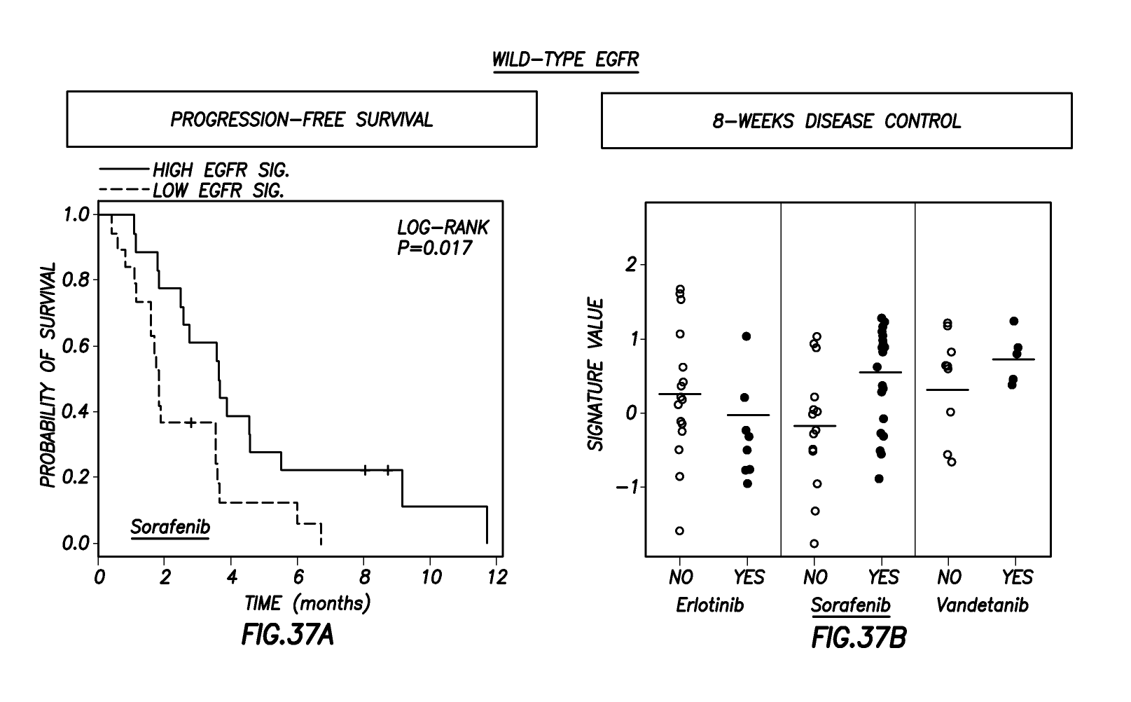

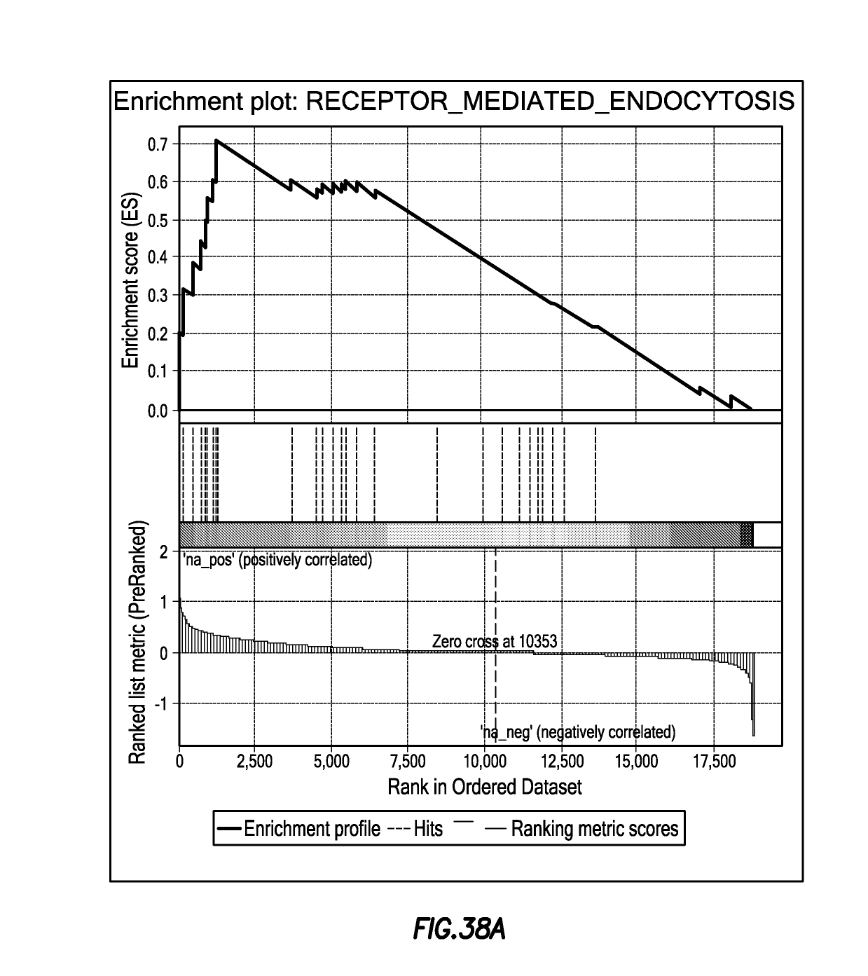

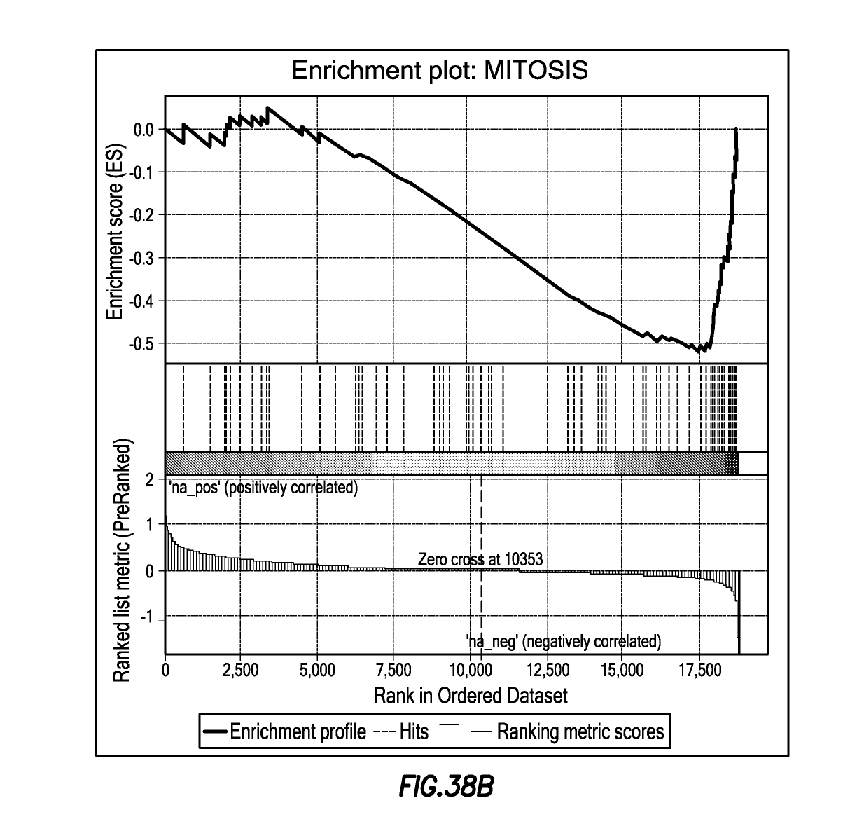

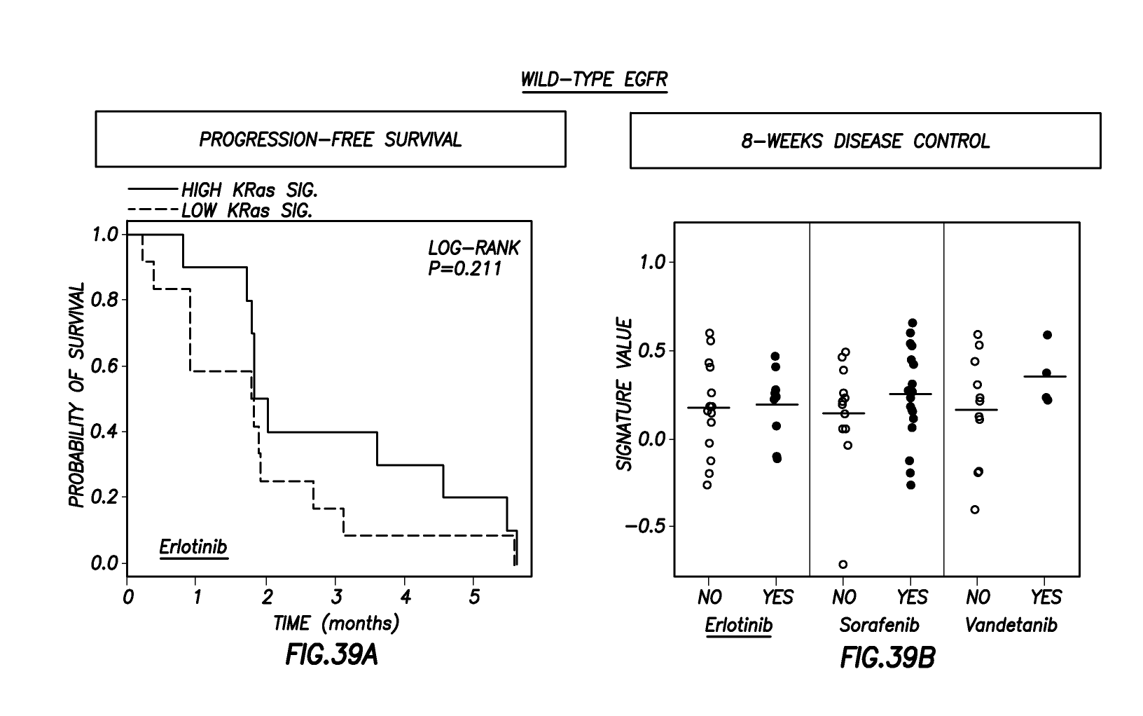

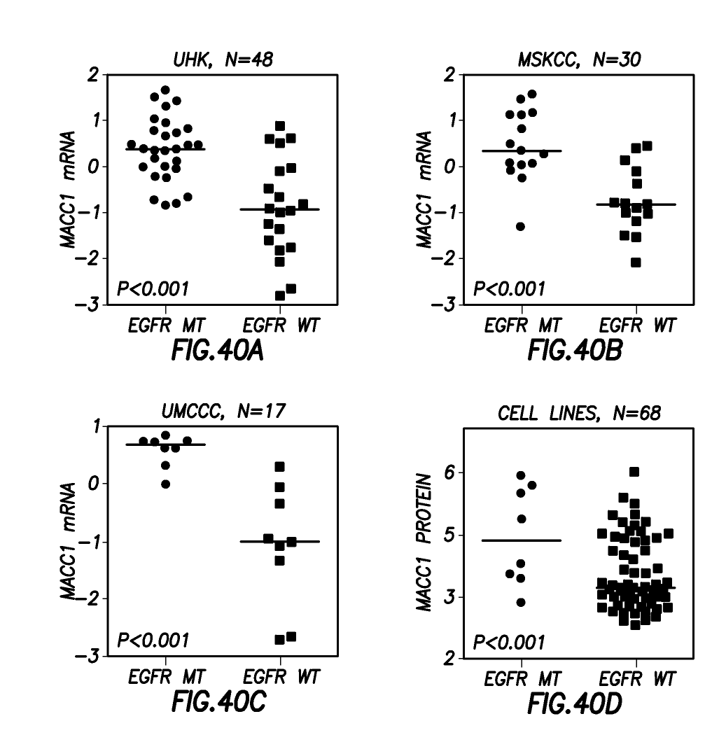

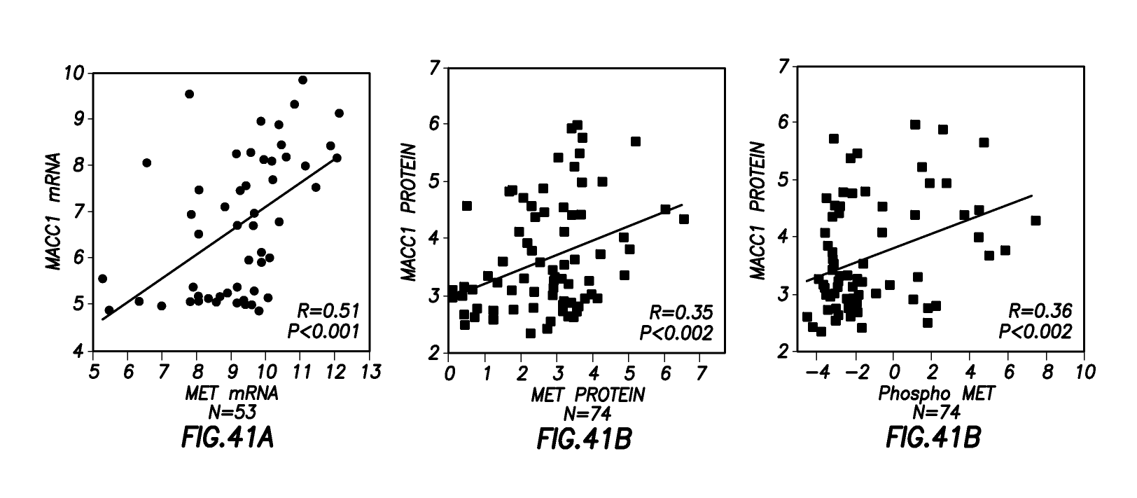

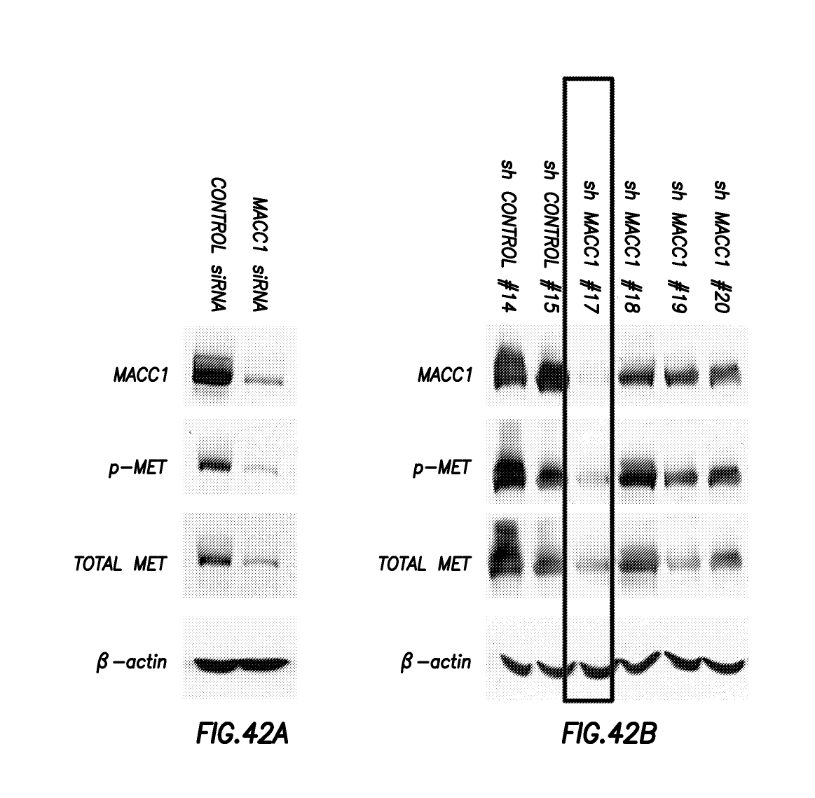

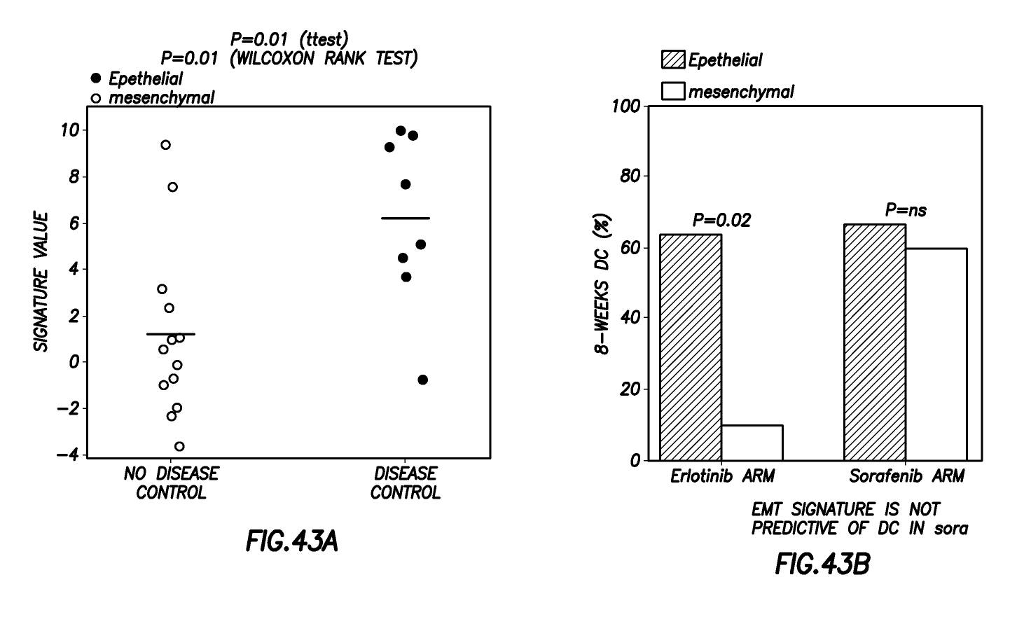

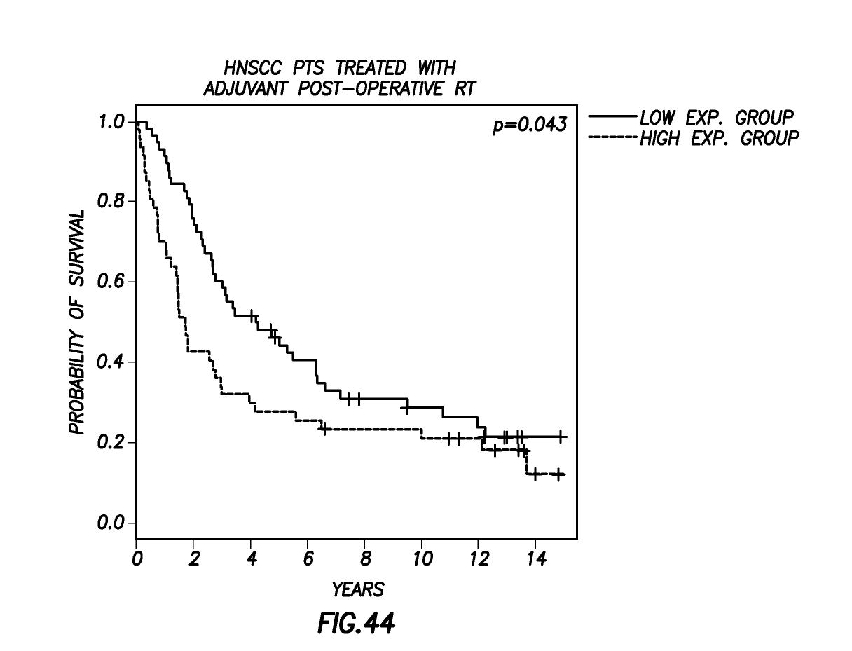

A five-gene signature for predicting benefit in patients with non-small cell lung cancer treated with erlotinib is provided herein. (FIG. 27) This gene signature as well as the individual markers can be used to identify which NSCLC patients are more likely to respond to erlotinib. This signature may help select patients that will experience greater benefit from a specific treatment regimen for NSCLC and other cancers, and potentially spare patients who are less likely to benefit from receiving toxic therapy. This signature may also be useful for predicting response to other EGFR inhibitors in NSCLC as well as other tumor types.

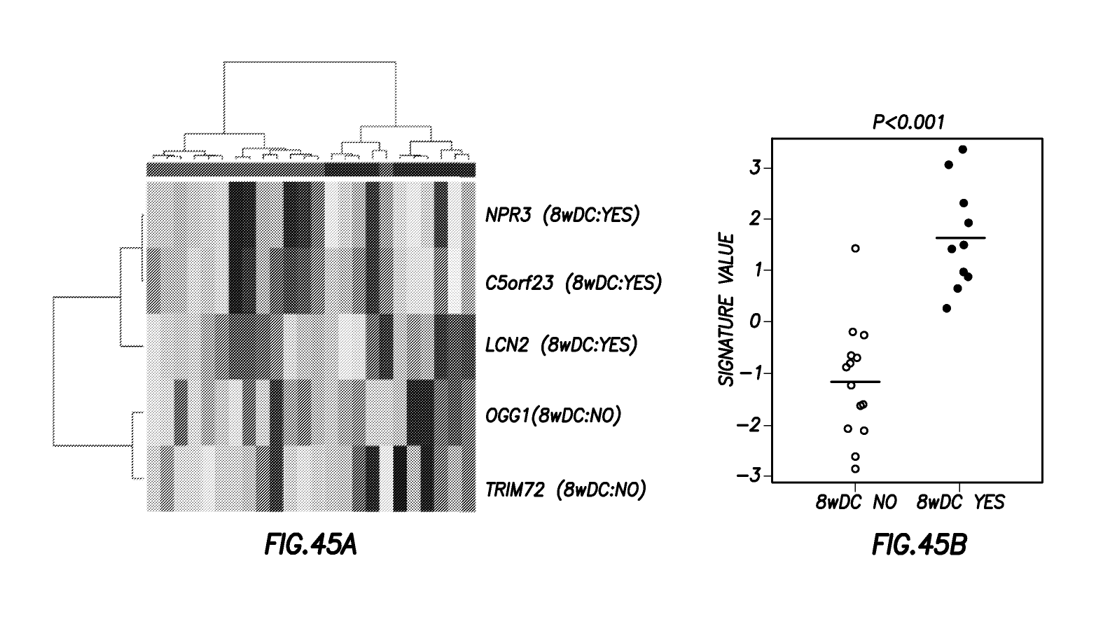

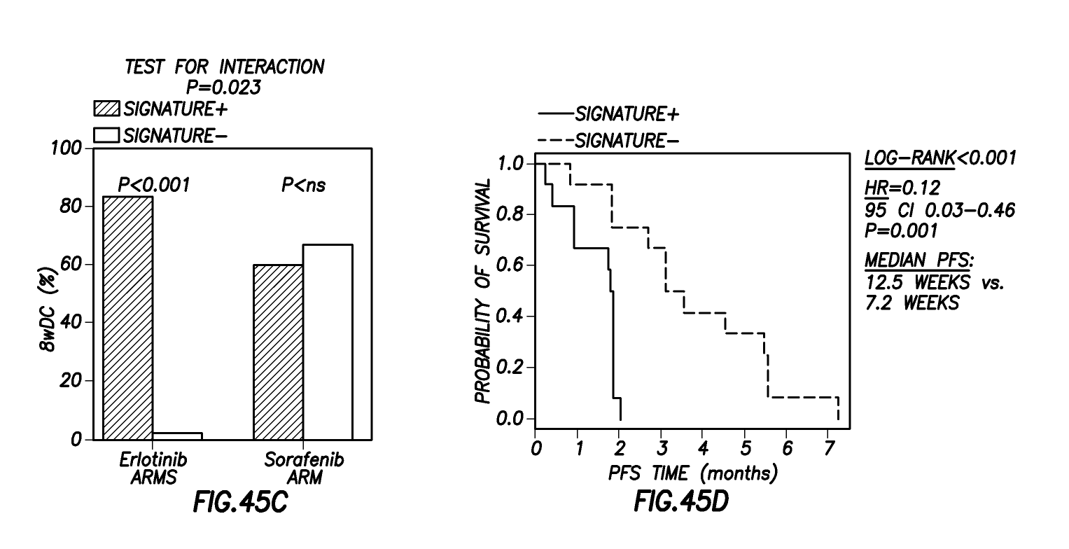

We conducted an analysis of tissue samples at MDACC from a trial of non-small cell lung cancer patients treated in the BATTLE trial. The analysis was conducted using the Affymetrix gene expression array platform. The five-gene signature was validated in a panel of NSCLC cell lines and predicts clinical response to erlotninib. (FIGS. 45 & 47)

We also investigated markers for identifying patients that would be most likely to benefit from erlotinib in patients with non-small cell lung cancer (NSCLC) treated in the BATTLE program. The Affymetrix platform was used to analyze gene expression from NSCLC patients treated in the BATTLE program. There were a total of 101 patients treated in the following arms: erlotinib (n=27), erlotinib+bexarotene (n=8), vandetinib (n=19) and sorafenib (n=47). A five gene signature that predicts clinical benefit (e.g. disease control) in patients that were EGFR and KRAS widtype was developed and validated in NSCLC cell lines. The genes including in the signature include the following probesets (gene name included if known): 219789_at (NPR3), 219790_s_at, 219054_at (C5orf23), 212531_at (LCN2), 205760_s_at (OGG1), and 205301_s_at. Of these genes, LCN2 has a very strong potential for predicting response to erlotinib on its own.

Despite a low response rate, erlotinib (E) improves survival in a subset of NSCLC patients with EGFR but there are no established markers for identifying patients likely to have clinical benefit.

### Material and Methods

We used pretreatment gene expression profiles (Affymetrix HG LOST) from 101 chemo-refractory patients in our Biomarkers-Integrated Approaches of Targeted Therapy for Lung Cancer Elimination (BATTLE) treated with E, E+bexarotene (EB), sorafenib (S), or vandetanib (V). 24 cases of with EGFR & KRAS tumors treated with E or EB were compared to train the signature (two-sided t-test), using the primary end-point of the trial[8-week disease control (8 with DC)]. Principal component (PC) analysis and a logistic regression model were used to develop the signature. Gene expression profiles from 108 NSCLC cell lines (Illumina), with available E IC50 (N=94) and DNA methylation profiling (N=66, Illumina), were used for in vitro studies.

### Results

113 genes were differentially expressed between patients with or without 8wDC (false discovery rate 30%; P=0.004). Leave-one-out cross validation with various gene list lengths produced the 5-gene signature, including lipocalin 2 (LCN2), with a specificity, sensitivity and accuracy of 80% to predict 8 with DC.

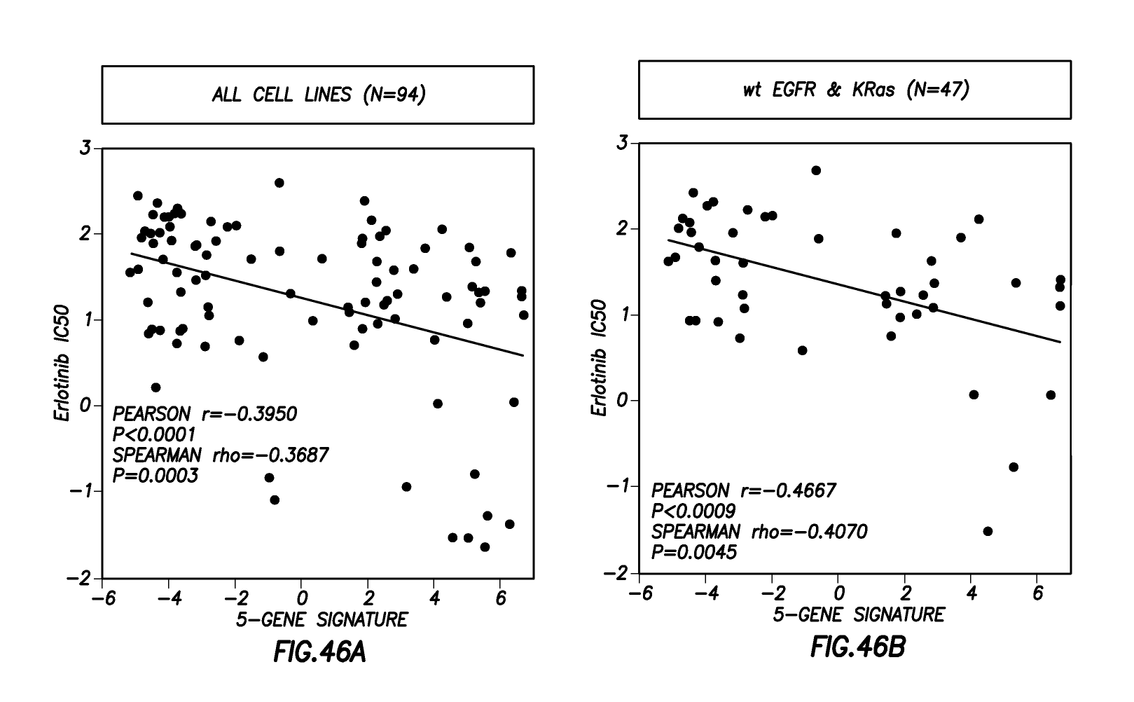

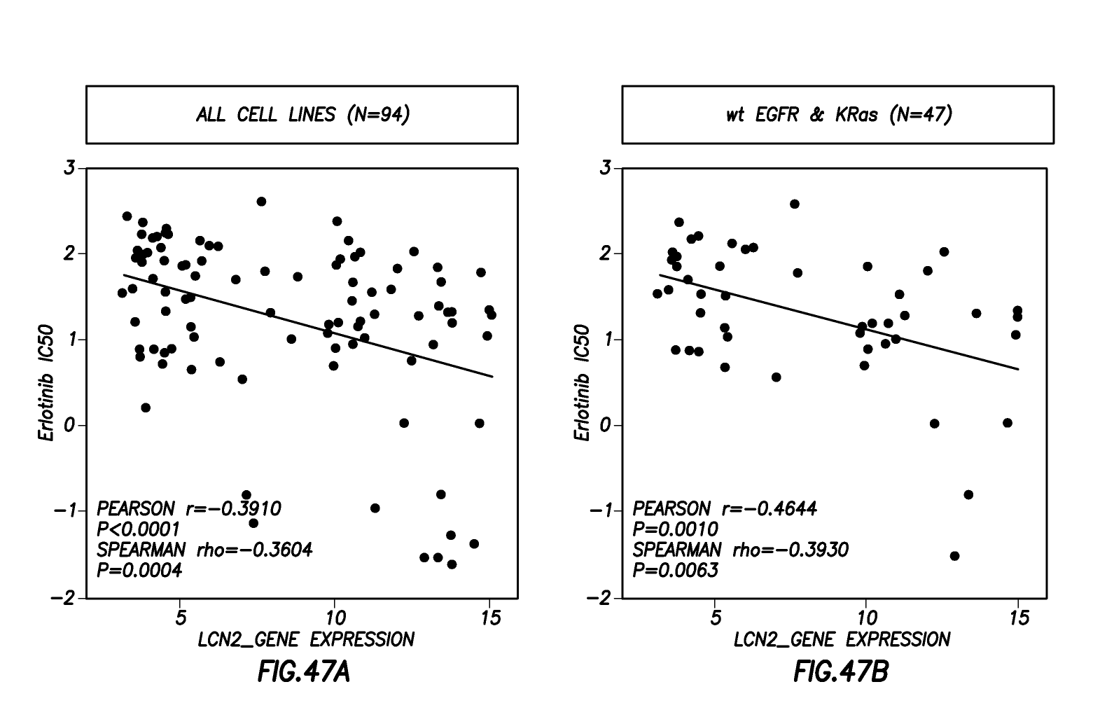

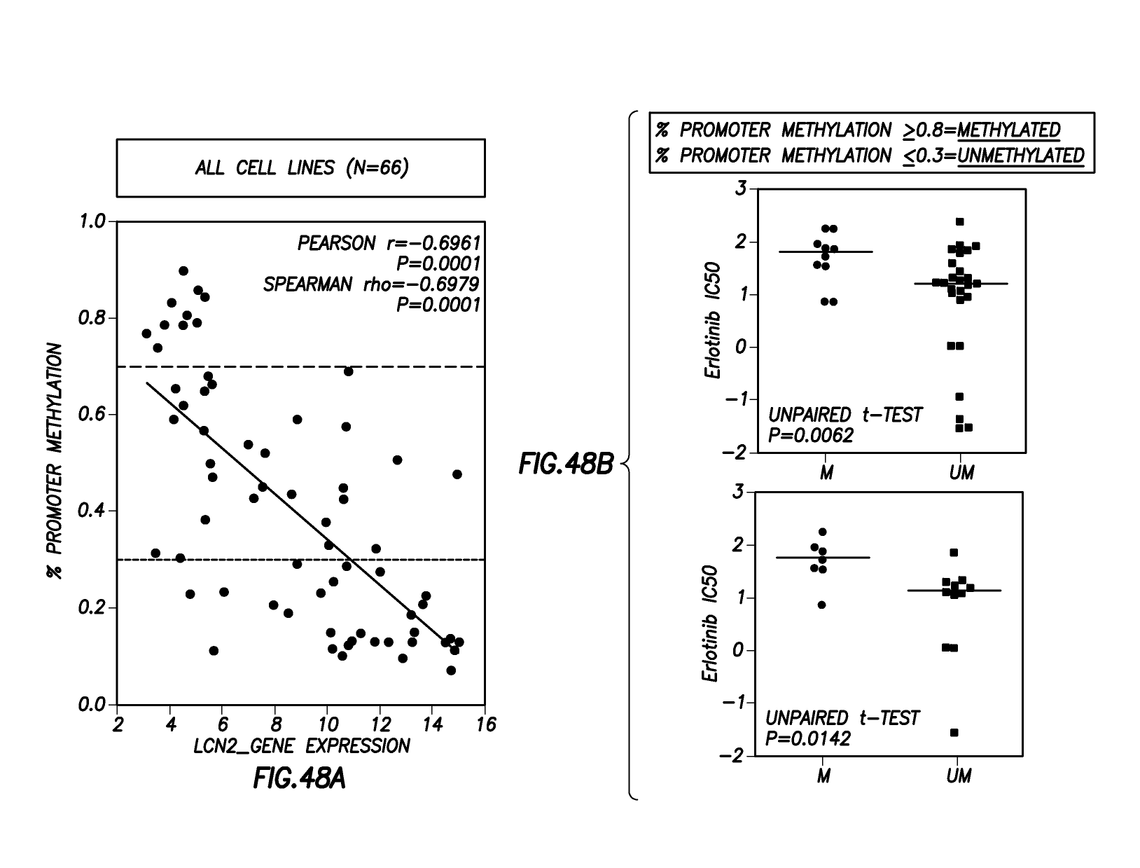

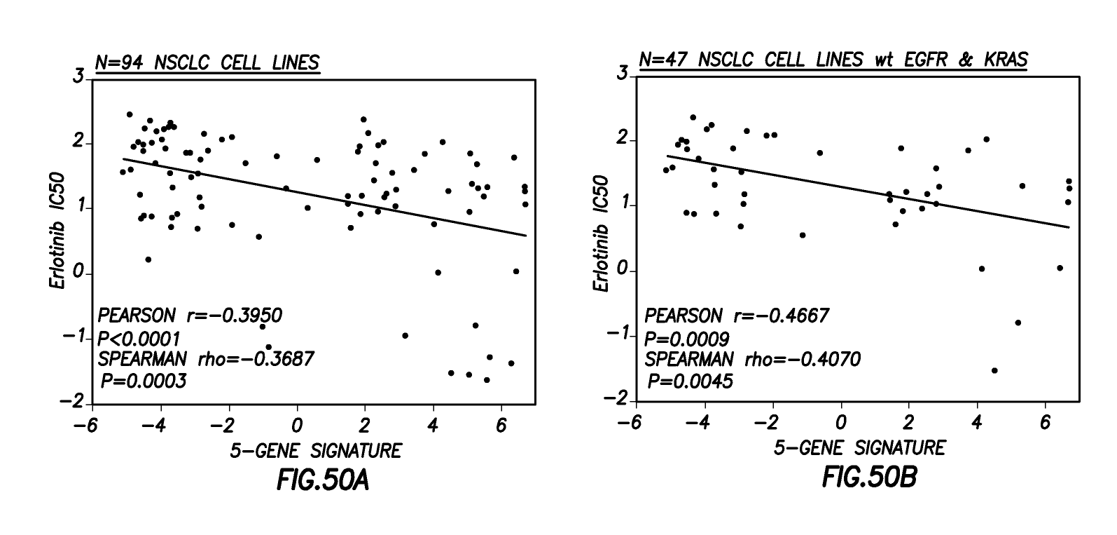

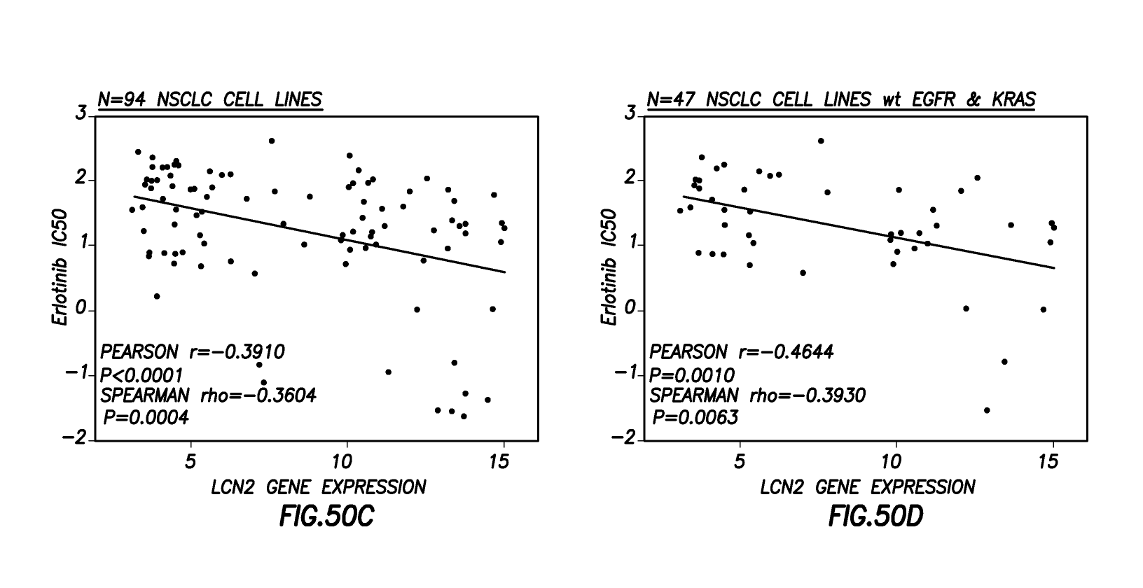

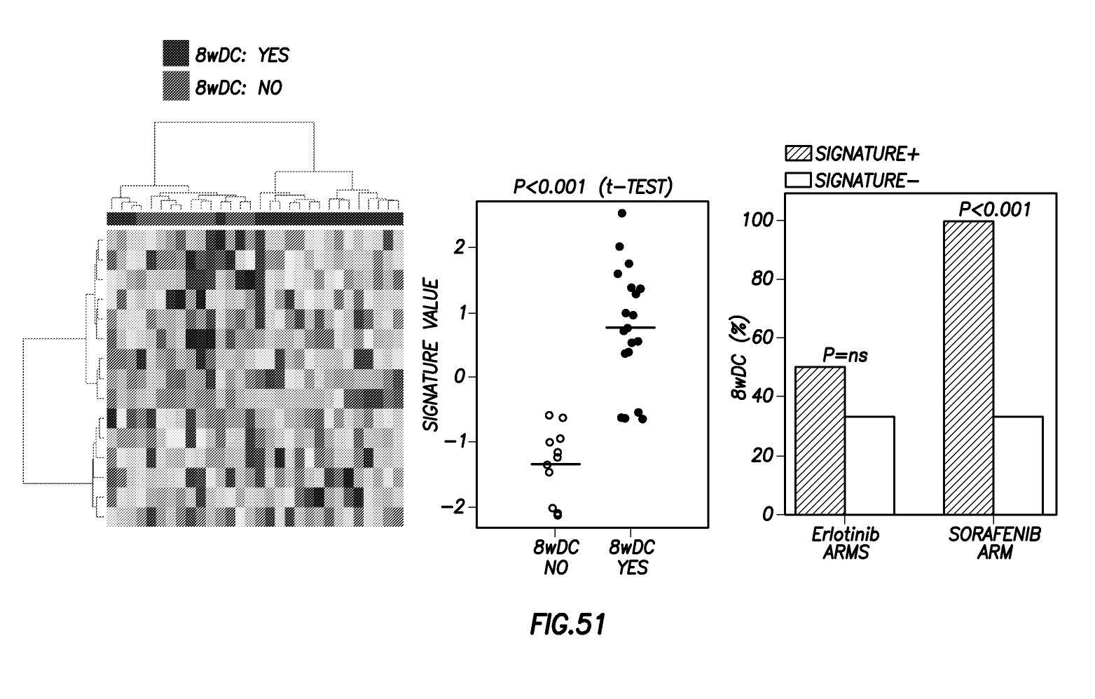

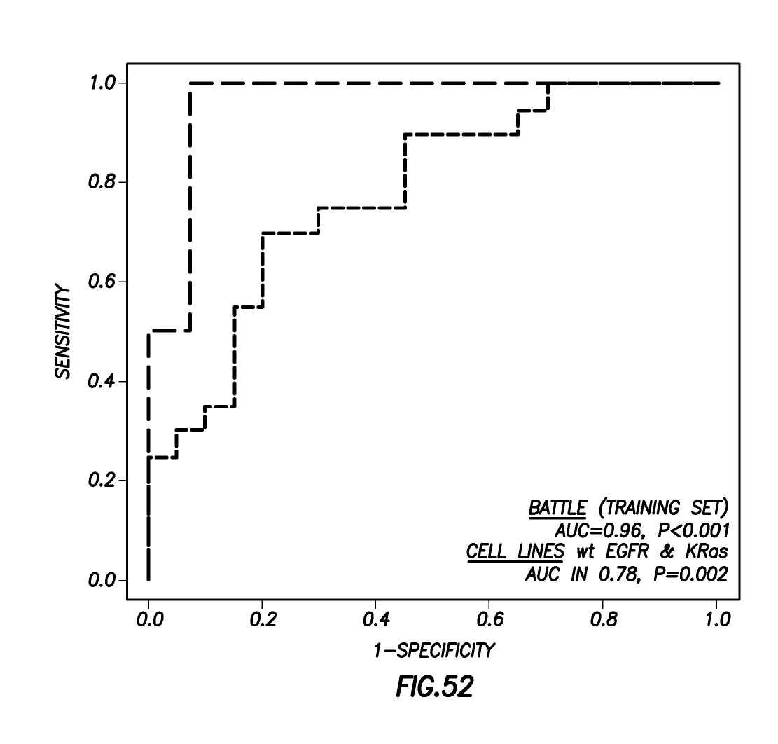

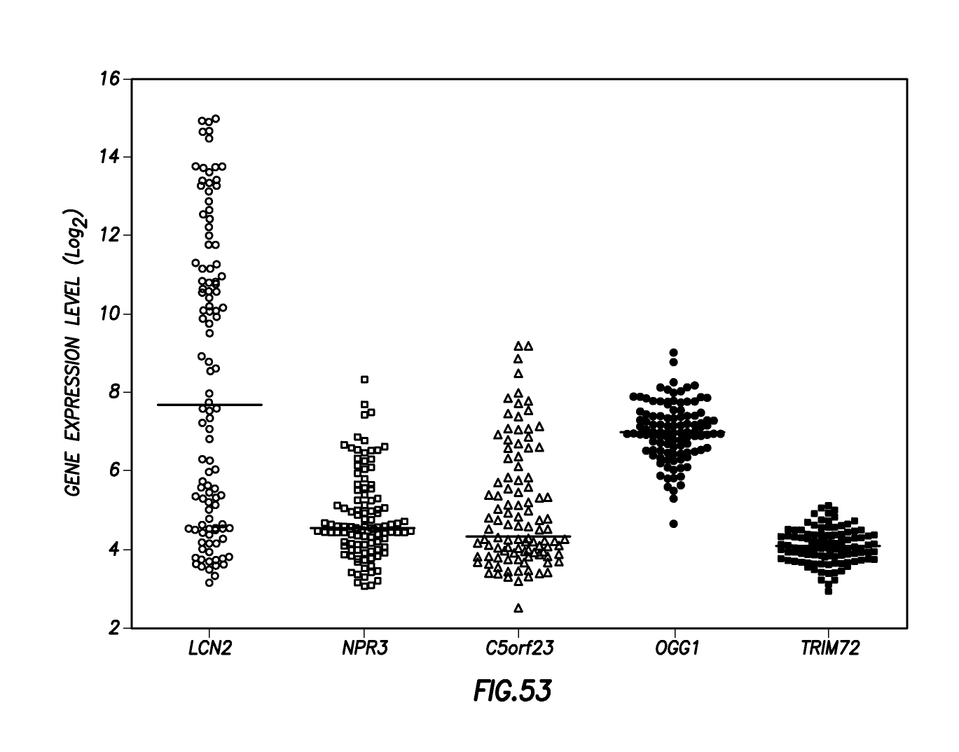

In patients treated with E or EB, using the median signature score, the 8 with DC rate in the signature-positive group was 83% compared with 0% in the signature-negative group; the signature did not predict 8wDC in patients treated with S or V (Mantel-Haenszel chi-squared test P=0.023). The improvement in 8 with DC in the signature-positive group translated to an increased progression-free survival (PFS) (hazard ratio=0.12, 95% confidence interval: 0.03-0.46, P=0.001; log-rank P=0.0004; median PFS: 12.5 weeks vs. 7.2 weeks). We tested the signature in an independent set of 47 with EGFR & KRAS cell lines. It predicted E sensitivity with an area under the curve of 78% (P=0.002). The first PC of the signature and the IC50 for E were correlated (r=−0.47, P=0.0009). In 108 NSCLC cell lines, LCN2 gene expression was bimodal and correlated with the IC50 for E (r=−0.46, P=0.001). Degree of methylation and expression level of LCN2 were inversely in with EGFR & KRAS NSCLC cells (r=−0.79, P<0.0001, N=33). Cell lines with completely unmethylated LCN2 were more sensitive to E compared to those with LCN2 full methylation (N=36) (P=0.006); the difference remained significant in with EGFR & KRAS cell lines (P=0.014). As noted above, FIGS. 45A, 45B, 45C and 45D show that the 5-gene signature including LCN2 is predictive of benefit for erlotinib in patients with wild-type EGFR. FIGS. 46A and 46B show the validation of the 5-gene signature in a large panel of cell lines. FIGS. 47A and 47B show that LCN2 is associated with erlotinib sensitivity in vitro in cells with wild-type EGFR. FIGS. 50A, 50B, 50C and 50D show that the 5-gene signature and LCN2 are associated with erlotinib sensitivity in vitro. FIG. 52 shows the results of the validation of the 5-gene signature in a large panel of cell lines. FIG. 53 shows the gene expression distribution of the 5 genes in 108 NSCLC cell lines.

### Conclusion

We identified a 5-gene signature predictive of PFS benefit in NSCLC patients with EGFR & KRAS treated with E, but not S or V. The signature was also predictive of E sensitivity in vitro. LCN2 was the strongest individual marker of sensitivity and may be epigenetically regulated.

### Example IV

### LCN2—A Predictive Marker

We have discovered that LCN2 is a predictive marker of benefit in patients with non-small cell lung cancer treated with EGFR inhibitors. This discovery could help select patients that will experience greater benefit from a specific treatment regimen for NSCLC and other cancers, and potentially spare patients who are less likely to benefit from receiving toxic therapy.

LCN2 as a biomarker could be used for the purpose of better selecting patients likely to respond to a given treatment, particularly for NSCLC patients treated with erlotinib or other EGFR inhibitor. Subsets of non-small-cell lung cancer (NSCLC) are currently defined in part by mutations in key oncogenic drivers such as EGFR and KRAS. EGFR inhibitors such as erlotinib prolong progression-free survival (PFS) and/or overall survival in previously treated NSCLC patients. Among these patients, the subset bearing EGFR mutations (˜10-15%) have a high likelihood of major objective tumor responses, while those bearing KRAS mutations (˜15-20%) are likely to be resistant to EGFR TKIs.

Patients bearing wild-type (wt) EGFR and KRAS do, however, appear to benefit overall from EGFR TKIs. For this group, which constitutes roughly two thirds of patients, there are currently no established markers to predict a clinical benefit from EGFR TKIs. Our hypothesis was that using a gene expression signature will allow the identification of a subgroup of patients with with EGFR&KRAS tumors that benefit from EGFR TKIs.

Therefore, we investigated markers for identifying patients that would be most likely to benefit from erlotinib in patients with non-small cell lung cancer (NSCLC) treated in the BATTLE program. The Affymetrix platform was used to analyze gene expression from NSCLC patients treated in the BATTLE program. There were a total of 101 patients treated in the following arms: erlotinib (n=27), erlotinib+bexarotene (n=8), vandetinib (n=19) and sorafenib (n=47).

As a result, and noted above, a five gene signature that predicts clinical benefit (e.g. disease control) in patients that were EGFR and KRAS wildtype was developed and validated in NSCLC cell lines. The genes included in the signature have the following probe sets (gene name included if known): 219789_at (NPR3), 219790_s_at, 219054_at (C5orf23), 212531_at (LCN2), 205760_s_at (OGG1), and 205301_s_at.

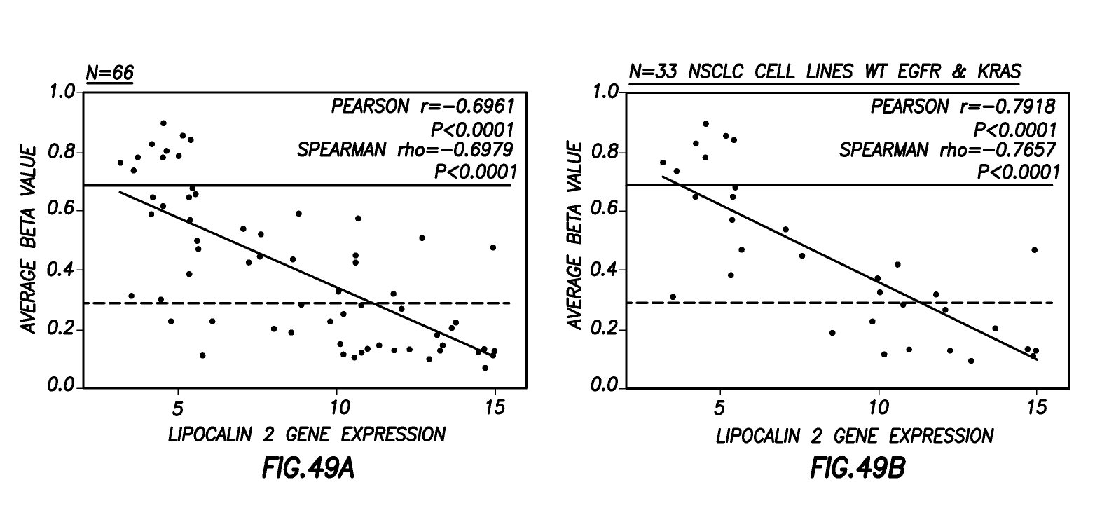

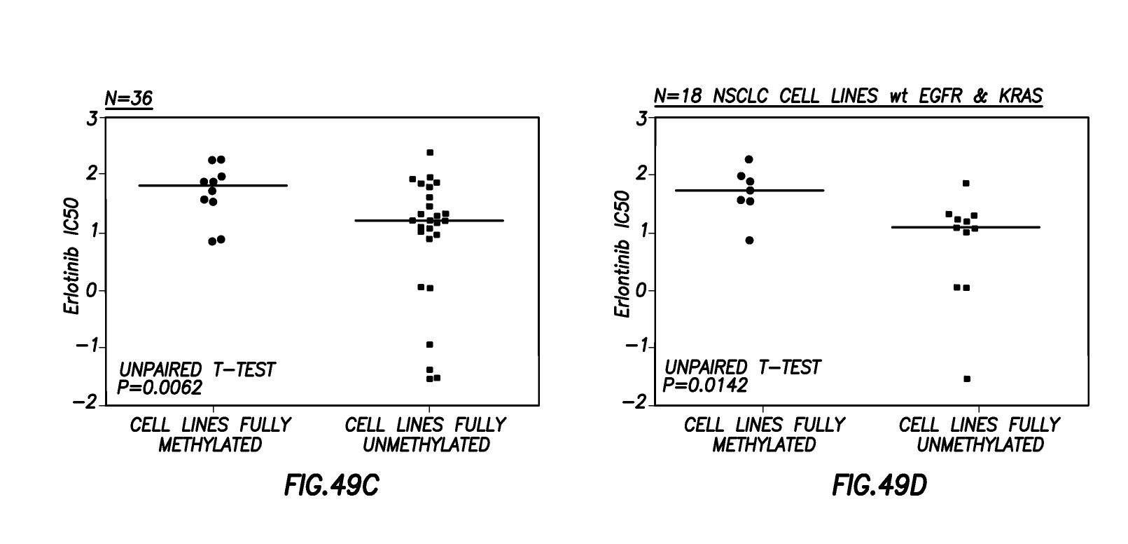

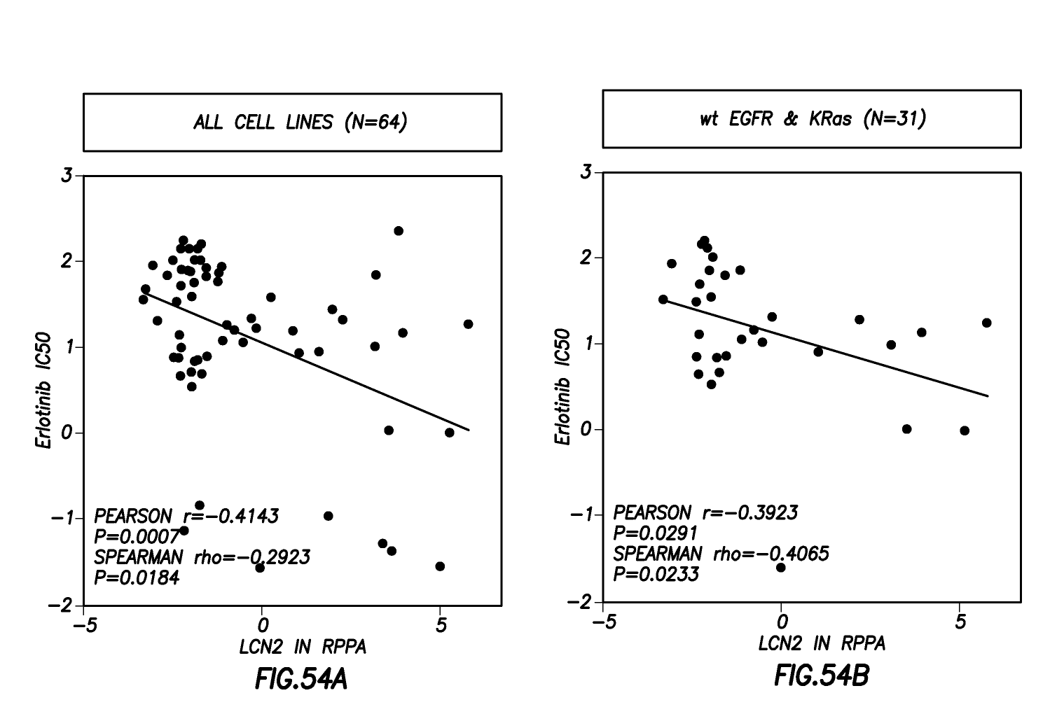

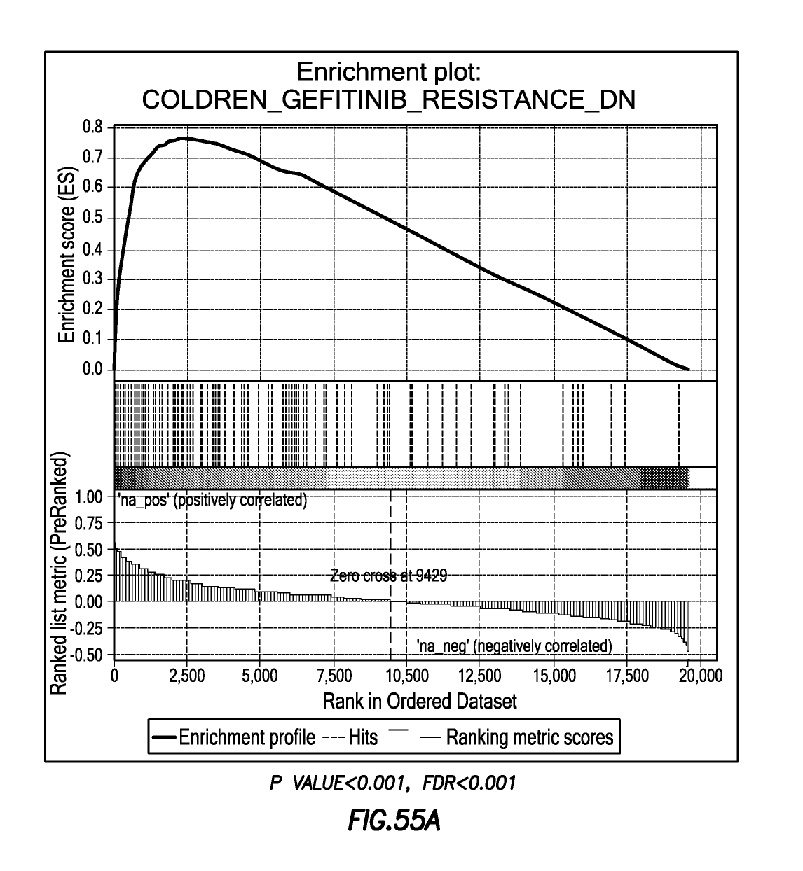

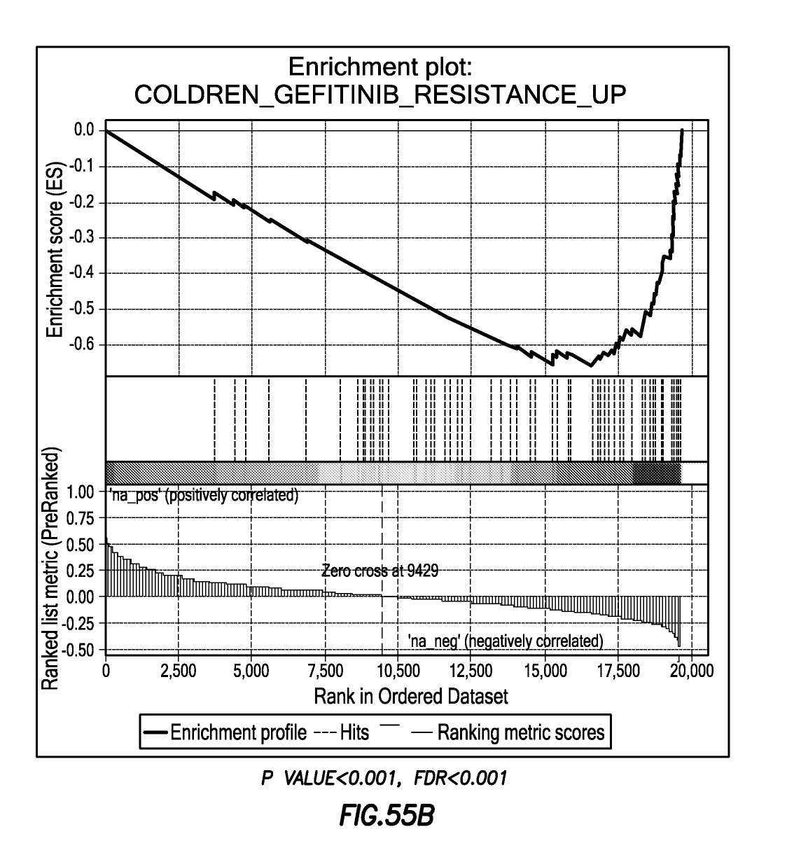

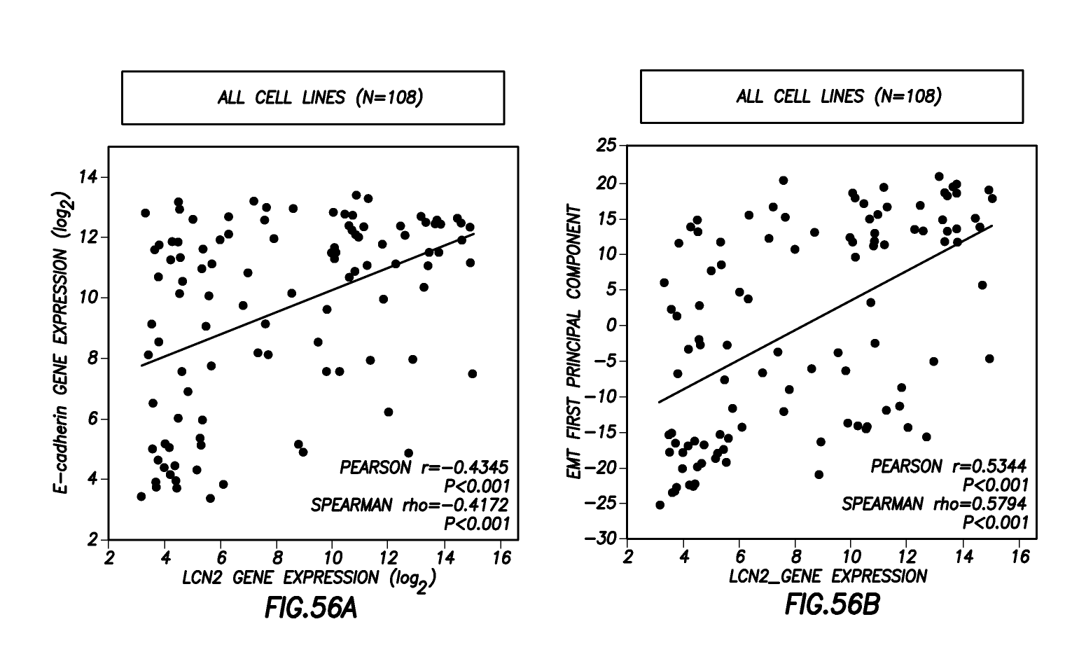

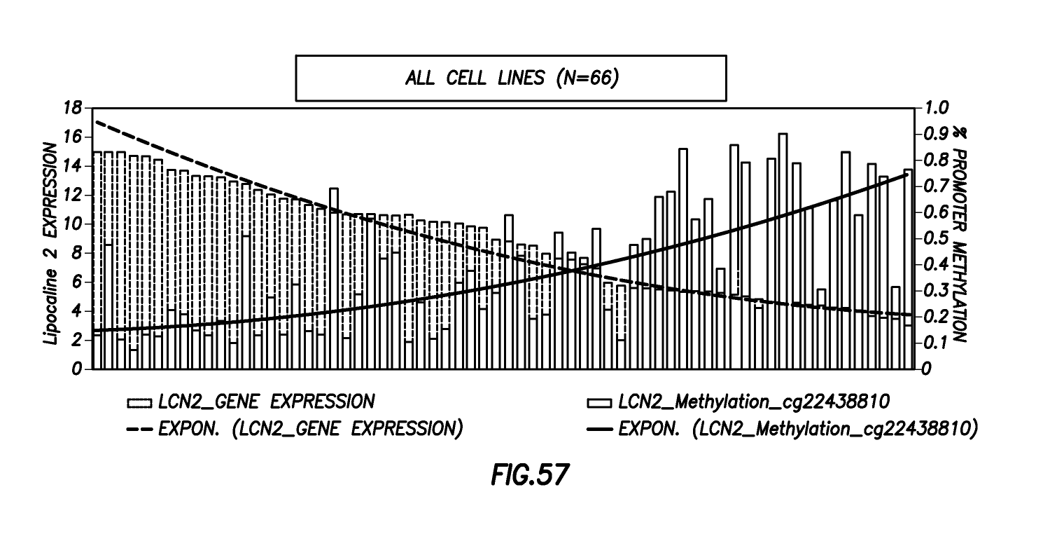

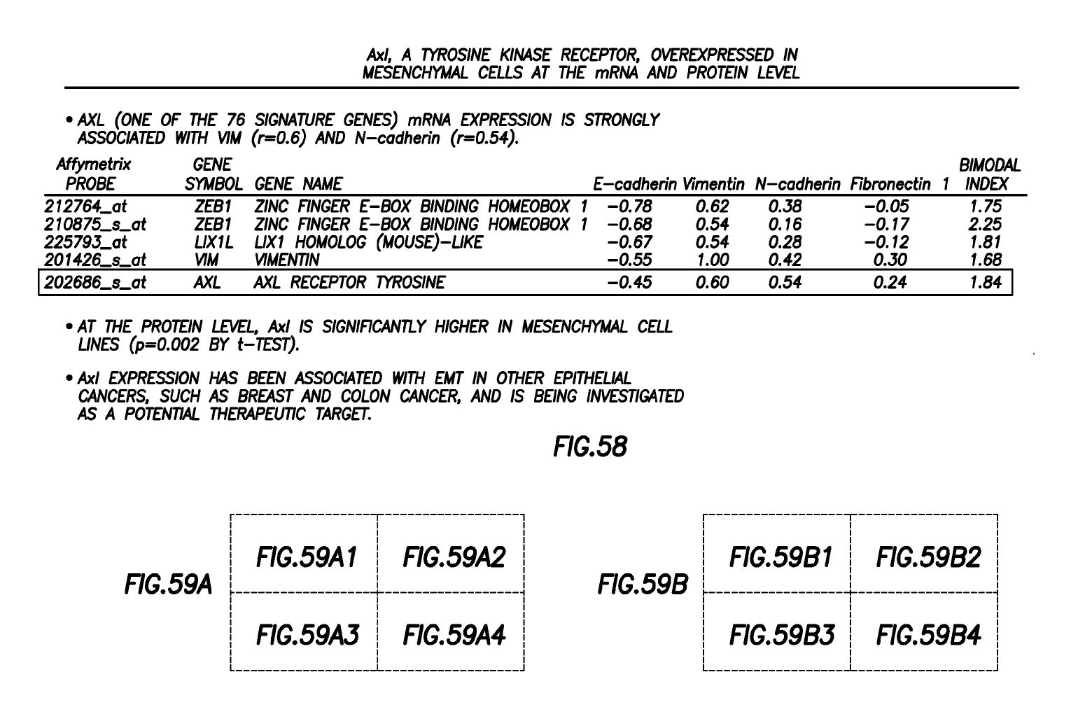

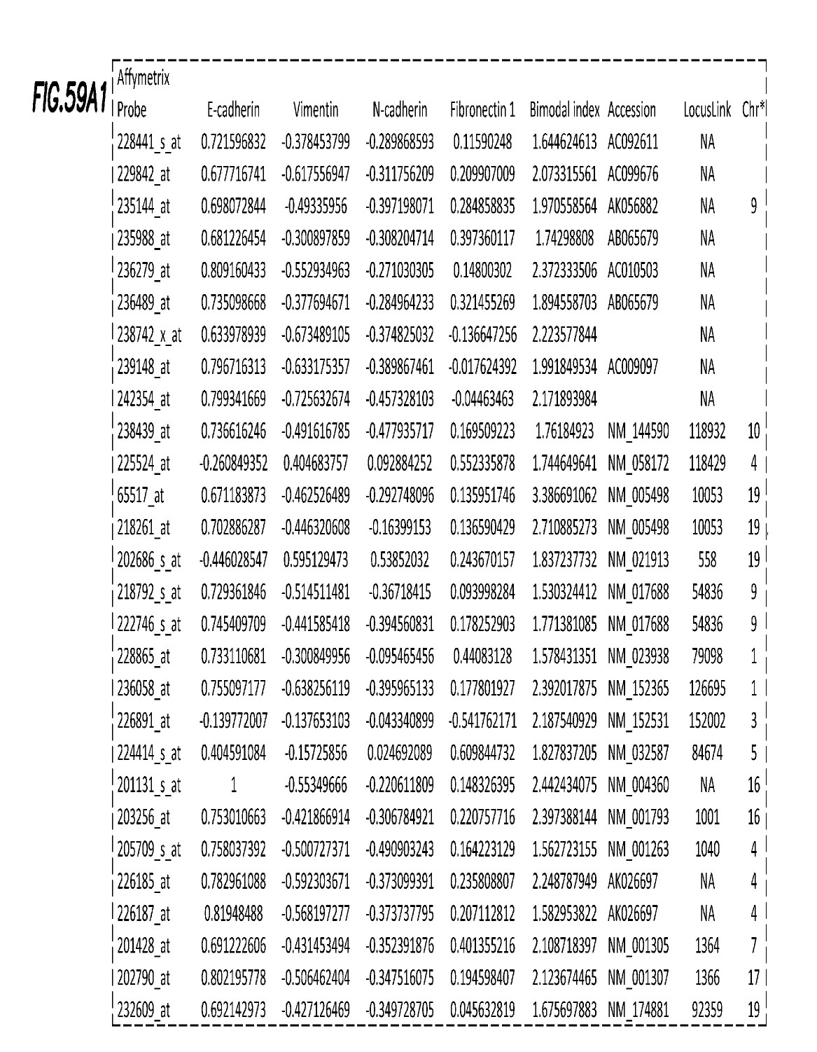

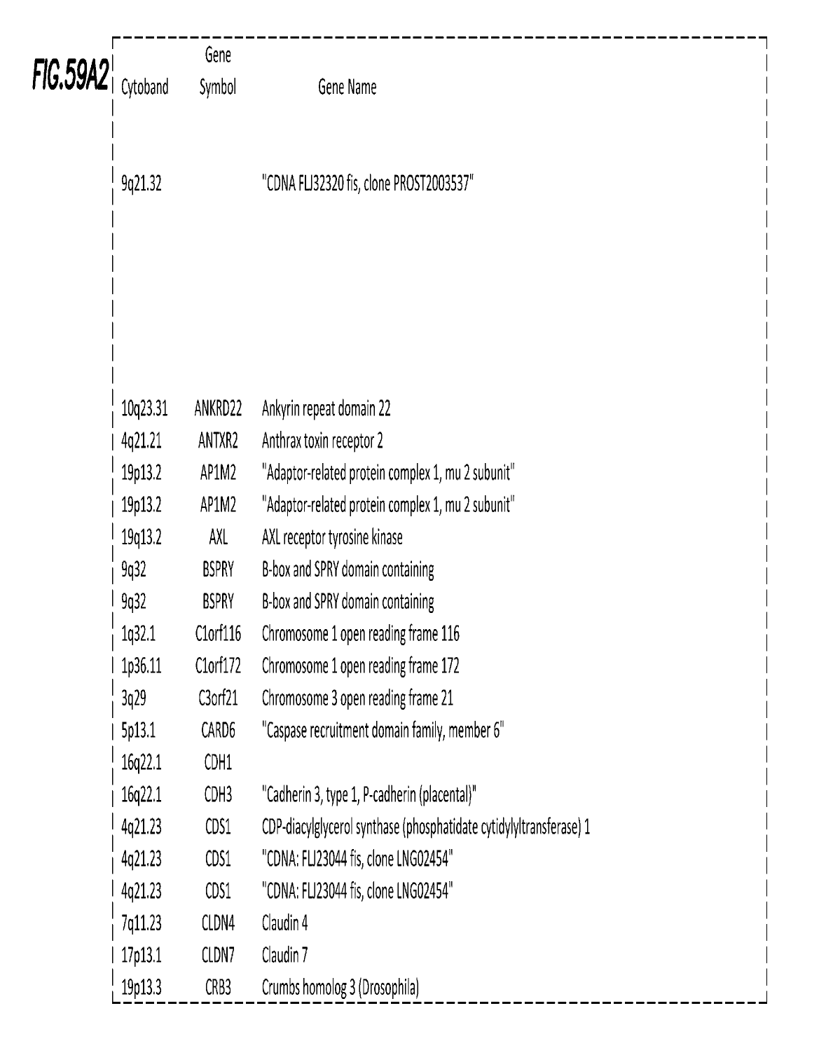

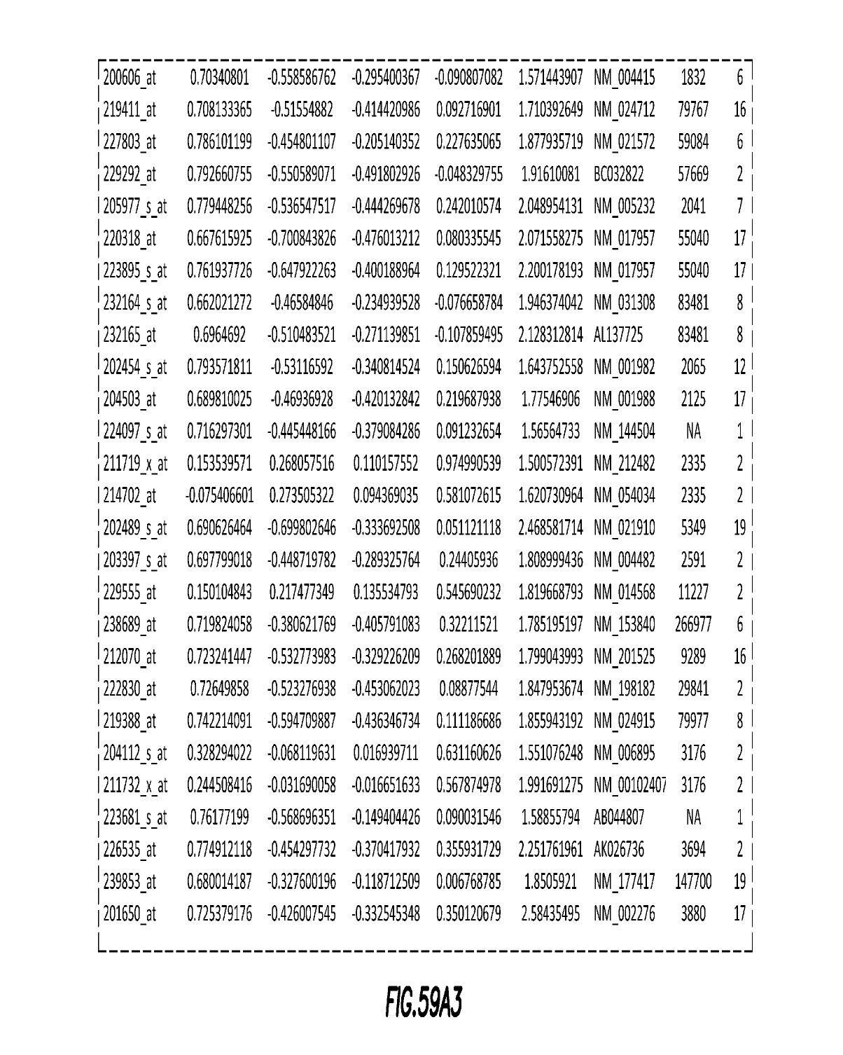

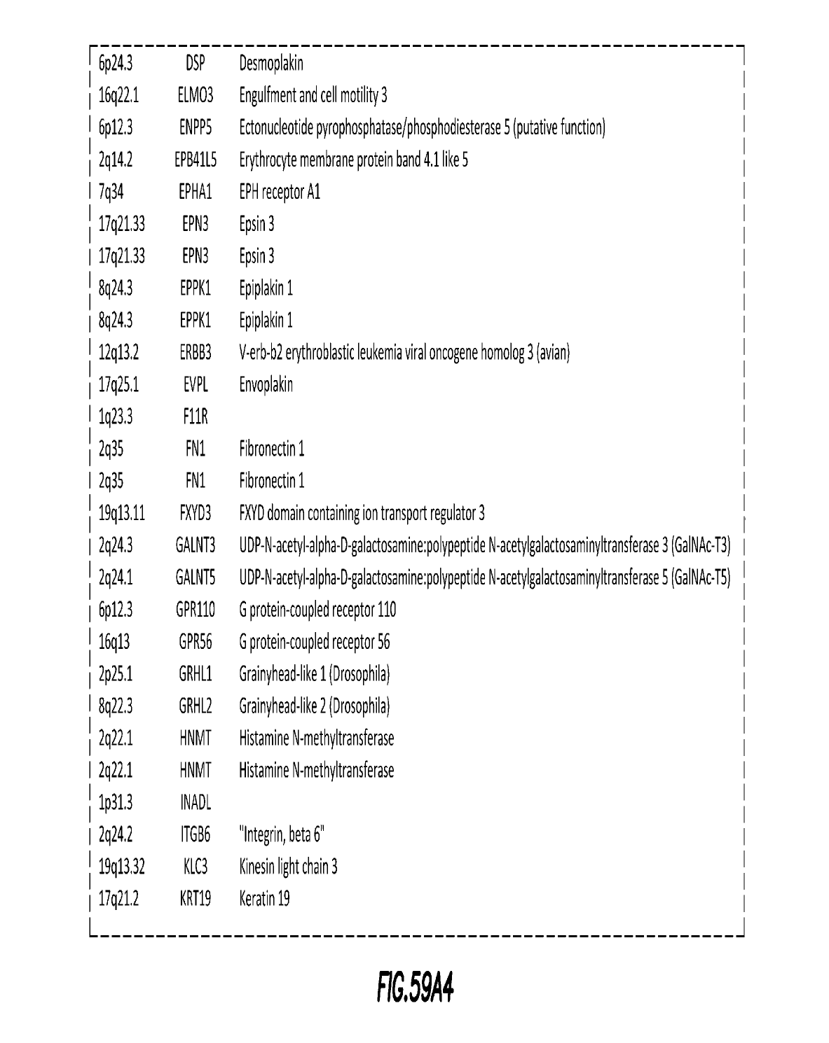

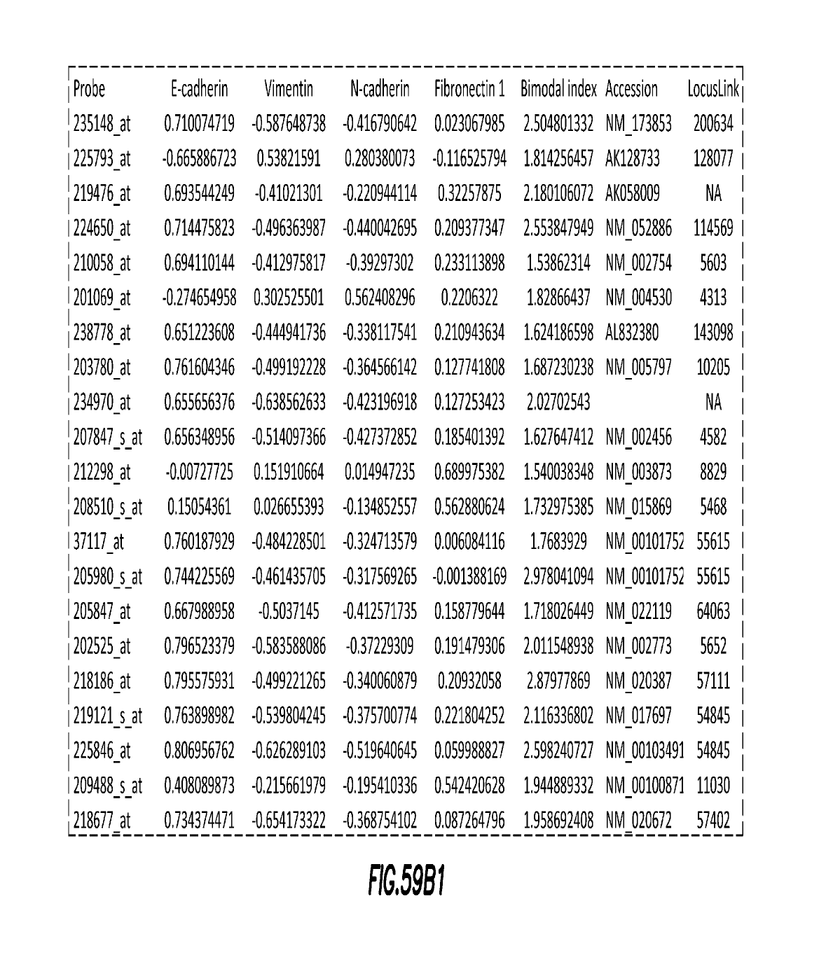

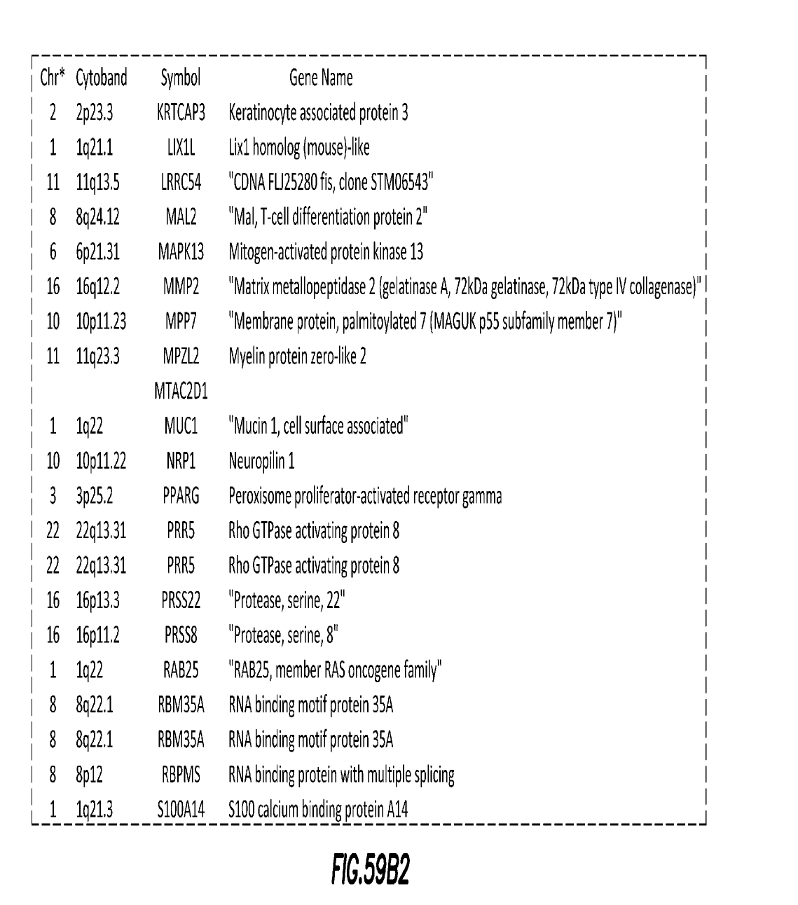

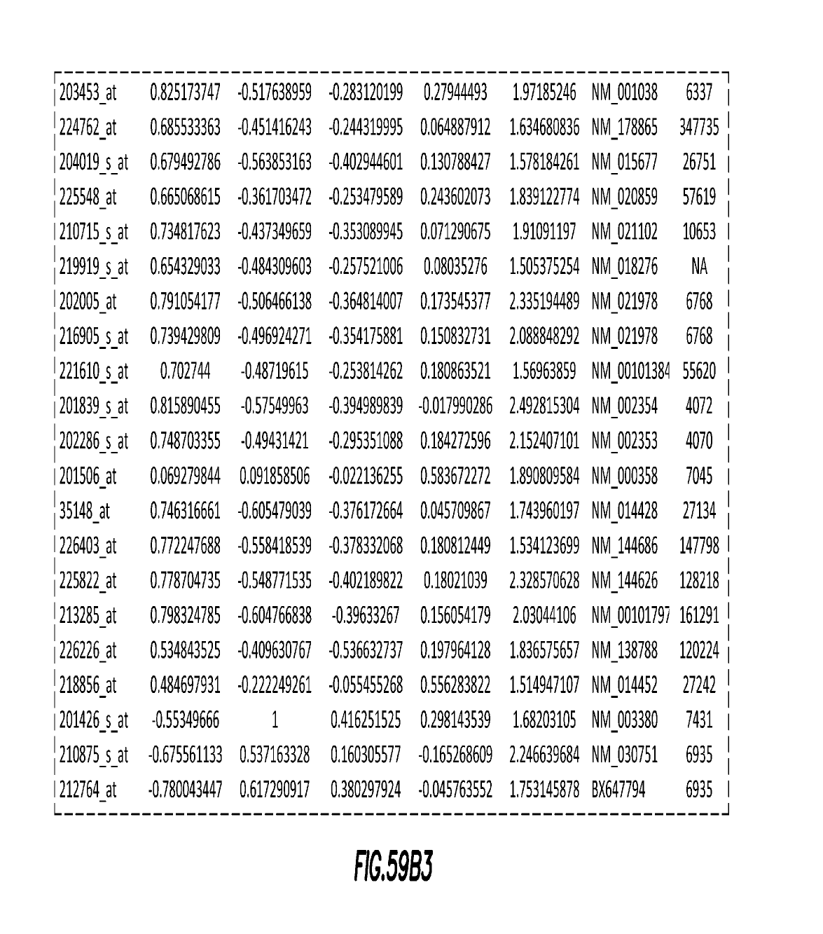

Furthermore, our data identified that one of the genes in this 5-gene signature, LCN2, is a potential biomarker for predicting response to EGFR inhibitors. LCN2 gene, protein and secreted form as detected in plasma was a biomarker of response. LCN2 is also a marker for EGFR inhibitors and other inhibitors of the EGFR family such as HER2 (trastuzumab) and an important marker for epithelial phenotype and PI3K activation and dependence. As noted above, FIGS. 49A, 49B, 49C and 49D show that LCN2 promoter methylation is associated with erlotinib sensitivity in vitro. FIGS. 54A and 54B show that LCN2 is correlated with sensitivity to erlotinib. FIGS. 55A and 55B show that genes correlated with lipocalin-2 (“LCN2”) are associated with sensitivity to gefitinib. FIGS. 56A and 56B show that LCN2 expression is correlated with E-cadherin and epithelial phenotype. FIG. 57 shows that LCN2 gene expression may be regulated through promoter methylation.

1.-7. (canceled)

8. A method for classifying an EMT status of a patient with non-small-cell lung cancer (NSCLC) comprising:
(a) obtaining a sample of the cancer;
(b) detecting an expression level in the sample of at least two nucleic acid molecules selected from the group consisting of the genes listed in Table 1; and
(c) comparing the expression level of the at least two nucleic acid molecules to a control level indicative of a known EMT status, wherein the comparison permits classifying the EMT status of the non-small-cell lung cancer in the patient as epithelial-like or mesenchymal-like.

9. The method of claim 8, wherein the at least two nucleic acid molecules are selected from the group consisting of: VIM, AXL, F11R, GPR56, ANKRD22, ERBB3, KRTCAP3, SH3YL1, TACSTD1, MAL2, SPINT2, SCINN1A, KRT19, TNFRSF21, MUC1, EPPK1, ST14, CLDN7, TMEM125, TMC4, S100A14, TMEM30B, PRSS8, GRHL2, EPHA1, RAB25, GPR110, CDS1, CDH3, C1orf116, MAPK13, ANTXR2, TGFB1, PPARG and HMNT.

10. The method of claim 8, wherein the control level comprises a level derived from corresponding transcripts in NSCLC samples of known classification.

11. A method of predicting a response to treatment with an EGFR inhibitor in a patient with NSCLC, the method comprising:
(a) classifying the EMT status of the cancer according to the method of claim 8; and
(b) predicting a response to treatment with the EGFR inhibitor, wherein if the cancer is classified as epithelial-like, then it is predicted as being sensitive to the EGFR inhibitor and wherein if the cancer is classified as mesenchymal-like, then it is predicted as being resistant to the EGFR inhibitor.

12. The method of claim 11, further comprising treating a patient having NSCLC predicted to be sensitive to the EGFR inhibitor with a therapeutically effective amount of the EGFR inhibitor.

13. A method of treating a patient with NSCLC comprising:
(a) selecting a patient determined to comprise an epithelial-like NSCLC according to the method of claim 8; and
(b) administering a therapeutically effective amount of an EGFR inhibitor to the patient.

14. The method of claim 13, wherein the EGFR inhibitor is erlotinib or gefitinib.

15. A method of treating a patient with NSCLC comprising:
(a) selecting a patient determined to comprise a mesenchymal-like NSCLC according to the method of claim 8; and
(b) administering a therapeutically effective amount of an Axl inhibitor to the patient.

16. The method of claim 15, wherein the Axl inhibitor is SGI-7079.

17. The method of claim 15, further comprising determining an Axl expression level in a sample of the NSCLC from the patient and administering a therapeutically effective amount of an EGFR inhibitor if the Axl expression level is increased relative to a reference level.

18. The method of claim 17, wherein the Axl expression level is an Axl protein level.

19. The method of claim 17, wherein the Axl expression level is an Axl mRNA level.

20. The method of claim 17, wherein the EGFR inhibitor is erlotinib or gefitinib.

21. A method of treating a patient with NSCLC comprising:
(a) obtaining a sample of the cancer;
(b) detecting an expression level in the sample of at least one gene selected from the group consisting of: NPR3, C4orf23, LCN2, OGG1, and TRIM72;
(c) comparing the expression level of the at least one gene to a control level indicative of EGFR inhibitor sensitivity, thereby classifying NSCLC as EGFR inhibitor sensitive or resistant; and
(d) administering a therapeutically effective amount of an EGFR inhibitor to the patient if the NSCLC is classified as EGFR inhibitor sensitive.

22. The method of claim 21, wherein the control level comprises a level derived from corresponding gene products in NSCLC samples of known EGFR inhibitor sensitivity.

23. The method of claim 21, wherein the at least one gene is LCN2.

24. The method of claim 23, wherein the expression level of LCN2 is determined by measuring an LCN2 mRNA level.

25. The method of claim 23, wherein the expression level of LCN2 is determined by measuring an LCN2 protein level.

26. The method of claim 21, wherein the EGFR inhibitor is erlotinib or gefitinib.
